SEQUENCE LISTING (170 sequences)

SEQ ID NO: 1 - DNA, 987 bp, Homo sapiens
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaa

SEQ ID NO: 2 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - DNA, 762 bp, Homo sapiens
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 4 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - DNA, 1008 bp, Mus musculus
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctaa

SEQ ID NO: 6 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 7 - DNA, 648 bp, Mus musculus
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaa

SEQ ID NO: 8 - protein, 215 aa, Mus musculus
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 9 - DNA, 1602 bp, Artificial Sequence
  source: /note=“Description of Artificial Sequence Synthetic fusion protein Construct ID 1481273”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacccctagcagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttccagcggacccgctcccacctcagccacggtcatctgccgcaaaaatgccagcattagcgtgcgggcccaggaccgctactatagctcatcttggagcgaatgggcatctgtgccctgctcctaa

SEQ ID NO: 10 - protein, 533 aa, Artificial Sequence
  source: /note=“Description of Artificial Sequence Synthetic fusion protein Construct ID 1481273”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTPSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGPAPTSATVICRKNASISVRAQDRYYSSSWSEWASVPCS

SEQ ID NO: 11 - DNA, 1602 bp, Artificial Sequence
  source: /note=“Description of Artificial Sequence Synthetic fusion protein Construct ID 1481272”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctggctccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttccccccagacccctggccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcctaa

SEQ ID NO: 12 - protein, 533 aa, Artificial Sequence
  source: /note=“Description of Artificial Sequence Synthetic fusion protein Construct ID 1481272”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASPQTPGPDTWSTPHSYFSLTFCVQVQGKSKREKKDRVFTDKTSATVICRKNASISVRAQDRYYSSSWSEWASVPCS

SEQ ID NO: 13 - DNA, 1623 bp, Artificial Sequence
  source: /note=“Description of Artificial Sequence Synthetic fusion protein Construct ID 1480533”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccagatcccccgtcagcggagacaatgccttccccgctcccacaggcagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 14 - protein, 540 aa, Artificial Sequence
  source: /note=“Description of Artificial Sequence Synthetic fusion protein Construct ID 1480533”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRSPVSGDNAFPAPTGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 15 - DNA, 1626 bp, Artificial Sequence
  source: /note=“Description of Artificial Sequence Synthetic fusion protein Construct ID 1480534”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccagatcccagcctgtgcctaccagggacctggaggtccccctcaccggaagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 16 - protein, 541 aa, Artificial Sequence
  source: /note=“Description of Artificial Sequence Synthetic fusion protein Construct ID 1480534”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRSQPVPTRDLEVPLTGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 17 - DNA, 1629 bp, Artificial Sequence
  source: /note=“Description of Artificial Sequence Synthetic fusion protein Construct ID 1480535”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccagatccggcacaccccctcagacaggcctggagaagcccacaggcacaggcagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 18 - protein, 542 aa, Artificial Sequence
  source: /note=“Description of Artificial Sequence Synthetic fusion protein Construct ID 1480535”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRSGTPPQTGLEKPTGTGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 19 - DNA, 1623 bp, Artificial Sequence
  source: /note=“Description of Artificial Sequence Synthetic fusion protein Construct ID 1480536”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccagcgatgtgacaggcaataccggaaacgctacctataccattaccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 20 - protein, 540 aa, Artificial Sequence
  source: /note=“Description of Artificial Sequence Synthetic fusion protein Construct ID 1480536”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSDVTGNTGNATYTITRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 21 - DNA, 1623 bp, Artificial Sequence
  source: /note=“Description of Artificial Sequence Synthetic fusion protein Construct ID 1480537”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggctcccccaaggacggacccgaaatccctcccacaggcggaaccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 22 - protein, 540 aa, Artificial Sequence
  source: /note=“Description of Artificial Sequence Synthetic fusion protein Construct ID 1480537”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSPKDGPEIPPTGGTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - DNA, 1629 bp, Artificial Sequence
  source: /note=“Description of Artificial Sequence Synthetic fusion protein Construct ID 1480538”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggcaggaacgctcccggaagccctcccacaggcaattacaaactggaacccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 24 - protein, 542 aa, Artificial Sequence
  source: /note=“Description of Artificial Sequence Synthetic fusion protein Construct ID 1480538”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGRNAPGSPPTGNYKLEPRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - DNA, 1632 bp, Artificial Sequence
  source: /note=“Description of Artificial Sequence Synthetic fusion protein Construct ID 1480539”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcccagaaaggctccgtgggattcacagaccctgaggtccaccaaagcacaaacctcagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 26 - protein, 543 aa, Artificial Sequence
  source: /note=“Description of Artificial Sequence Synthetic fusion protein Construct ID 1480539”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSQKGSVGFTDPEVHQSTNLRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 27 - DNA, 1623 bp, Artificial Sequence
  source: /note=“Description of Artificial Sequence Synthetic fusion protein Construct ID 1480540”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggcaatgtgcctgagctgcccgataccacagagcatagcaggaccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 28 - protein, 540 aa, Artificial Sequence
  source: /note=“Description of Artificial Sequence Synthetic fusion protein Construct ID 1480540”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGNVPELPDTTEHSRTRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 29 - DNA, 1635 bp, Artificial Sequence
  source: /note=“Description of Artificial Sequence Synthetic fusion protein Construct ID 1480541”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggcaggagccatcccgtccagccttaccctggcgctttcgtcaaggaacccattcccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 30 - protein, 544 aa, Artificial Sequence
  source: /note=“Description of Artificial Sequence Synthetic fusion protein Construct ID 1480541”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGRSHPVQPYPGAFVKEPIPRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - DNA, 1623 bp, Artificial Sequence
  source: /note=“Description of Artificial Sequence Synthetic fusion protein Construct ID 1480542”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcccccgaaagaaaagagaggatctccgagcaaacctatcagctgtccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 32 - protein, 540 aa, Artificial Sequence
  source: /note=“Description of Artificial Sequence Synthetic fusion protein Construct ID 1480542”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSPERKERISEQTYQLSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 33 - DNA, 1623 bp, Artificial Sequence
  source: /note=“Description of Artificial Sequence Synthetic fusion protein Construct ID 1480543”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggtggcggtggctcgggcggtggtgggtcgggtggcggcggatctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 34 - protein, 540 aa, Artificial Sequence
  source: /note=“Description of Artificial Sequence Synthetic fusion protein Construct ID 1480543”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 35 - DNA, 1599 bp, Artificial Sequence
  source: /note=“Description of Artificial Sequence Synthetic fusion protein Construct ID 1480544”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggtggcggtggcggcggatctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 36 - protein, 532 aa, Artificial Sequence
  source: /note=“Description of Artificial Sequence Synthetic fusion protein Construct ID 1480544”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 37 - DNA, 1653 bp, Artificial Sequence
  source: /note=“Description of Artificial Sequence Synthetic fusion protein Construct ID 1480545”
atgtgtccagcgcgcagcctcctccttgtggctaccctggtcctcctggaccacctcagtttggccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttccagatccgacgtgaactccagaacaggccctagcggagccacaccccctagcggcaacccctacacaatcacaggcatatgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcctaa

SEQ ID NO: 38 - protein, 550 aa, Artificial Sequence
  source: /note=“Description of Artificial Sequence Synthetic fusion protein Construct ID 1480545”
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASRSDVNSRTGPSGATPPSGNPYTITGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 39 - DNA, 1641 bp, Artificial Sequence
  source: /note=“Description of Artificial Sequence Synthetic fusion protein Construct ID 1480546”
atgtgtccagcgcgcagcctcctccttgtggctaccctggtcctcctggaccacctcagtttggccagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcccccgctcccacaccctccaacggaagccctaaggatggccctgagattccccctaccggaggcatatgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctcctaa

SEQ ID NO: 40 - protein, 546 aa, Artificial Sequence
  source: /note=“Description of Artificial Sequence Synthetic fusion protein Construct ID 1480546”
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASPAPTPSNGSPKDGPEIPPTGGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 41 - protein, 3 aa, Artificial Sequence
  source: /note=“Description of Artificial Sequence Synthetic linker”
TPS

SEQ ID NO: 42 - protein, 5 aa, Artificial Sequence
  source: /note=“Description of Artificial Sequence Synthetic linker”
SGPAP

SEQ ID NO: 43 - protein, 326 aa, Unknown
  source: /note=“Description of Unknown Wild-type IL-12 p40 polypeptide”
MCHQQLVISWFSLVFLDLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 44 - protein, 324 aa, Artificial Sequence
  source: /note=“Description of Artificial Sequence Synthetic destabilized #2 IL-12 p40 polypeptide”
MCHQQLVISWFSLVFLDLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSDAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 45 - protein, 197 aa, Unknown
  source: /note=“Description of Unknown Wild-type IL-12 p35 polypeptide”
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 46 - protein, 197 aa, Artificial Sequence
  source: /note=“Description of Artificial Sequence Synthetic destabilized #2 IL-12 p35 polypeptide”
RNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDKVMSYLNAS

SEQ ID NO: 47 - protein, 530 aa, Artificial Sequence
  source: /note=“Description of Artificial Sequence Synthetic polypeptide”
  source: /note=“ Wild-type (non-destabilized) IL-12 with the p40 and p35 polypeptides linked as single chain fusion protein by a Gly-Ser linker (no thrombin target site)”
MCHQQLVISWFSLVFLDLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 48 - DNA, 1599 bp, Artificial Sequence; identical to SEQ ID NO: 35
  source: /note=“Description of Artificial Sequence Synthetic polynucleotide”
  source: /note=“ DNA sequence encoding Wild-type (non-destabilized) IL-12 with the p40 and p35 polypeptides linked as single chain fusion protein by a Gly-Ser linker (no thrombin target site)”

SEQ ID NO: 49 - protein, 528 aa, Artificial Sequence
  source: /note=“Description of Artificial Sequence Synthetic polypeptide”
  source: /note=“Destabilized IL-12 #2 (DSIL-12 #2) having AA substituted p40 and p35 polypeptides linked as a single chain fusion protein by a thrombin target site linker”
MCHQQLVISWFSLVFLDLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSDAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDKVMSYLNAS

SEQ ID NO: 50 - DNA, 1599 bp, Artificial Sequence
  source: /note=“Description of Artificial Sequence Synthetic polynucleotide”
  source: /note=“DNA sequence encoding Destabilized IL-12 #2 (DSIL-12 #2) having AA substituted p40 and p35 polypeptides linked as a single chain fusion protein by a thrombin target site linker”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgccagcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccctggtgcccagaggcagctctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagtagcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgataaggtgatgagctatctgaatgcttcctaa

SEQ ID NO: 51 - protein, 530 aa, Artificial Sequence
  source: /note=“Description of Artificial Sequence Synthetic polypeptide”
  source: /note=“Destabilized IL-12 #4 (DSIL-12 #4) having p40 and AA substituted p35 polypeptides linked as a single chain fusion protein by a thrombin target site linker”
MCHQQLVISWFSLVFLDLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDKVMSYLNAS

SEQ ID NO: 52 - DNA, 1599 bp, Artificial Sequence
  source: /note=“Description of Artificial Sequence Synthetic polynucleotide”
  source: /note=“DNA sequence encoding Destabilized IL-12 #4 (DSIL-12 #4) having p40 and AA substituted p35 polypeptides linked as a single chain fusion protein by a thrombin target site linker”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccctggtgcccagaggcagctctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgataaggtgatgagctatctgaatgcttcctaa

SEQ ID NO: 53 - protein, 528 aa, Artificial Sequence
  source: /note=“Description of Artificial Sequence Synthetic polypeptide”
  source: /note=“Destabilized IL-12 #5 (DSIL-12 #5) having AA substituted p40 and p35 polypeptides linked as a single chain fusion protein by a thrombin target site linker”
MCHQQLVISWFSLVFLDLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSDAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 54 - DNA, 1599 bp, Artificial Sequence
  source: /note=“Description of Artificial Sequence Synthetic polynucleotide”
  source: /note=“DNA sequence encoding Destabilized IL-12 #5 (DSIL-12 #5) having AA substituted p40 and p35 polypeptides linked as a single chain fusion protein by a thrombin target site linker”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgccagcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccctggtgcccagaggcagctctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagtagcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaa

SEQ ID NO: 55 - protein, 528 aa, Artificial Sequence
  source: /note=“Description of Artificial Sequence Synthetic polypeptide”
  source: /note=“Destabilized IL-12 #1 (DSIL-12 #1) having AA substituted p40 and p35 polypeptides linked as a single chain fusion protein by a thrombin target site linker”
MCHQQLVISWFSLVFLDLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSDAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDAVMSYLNAS

SEQ ID NO: 56 - DNA, 1599 bp, Artificial Sequence
  source: /note=“Description of Artificial Sequence Synthetic polynucleotide”
  source: /note=“DNA sequence encoding Destabilized IL-12 #1 (DSIL-12 #1) having AA substituted p40 and p35 polypeptides linked as a single chain fusion protein by a thrombin target site linker”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgccagcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccctggtgcccagaggcagctctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagtagcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatgccgtgatgagctatctgaatgcttcctaa

SEQ ID NO: 57 - protein, 530 aa, Artificial Sequence
  source: /note=“Description of Artificial Sequence Synthetic polypeptide”
  source: /note=“Destabilized IL-12 #3 (DSIL-12 #3) having AA substituted p40 and p35 polypeptides linked as a single chain fusion protein by a thrombin target site linker”
MCHQQLVISWFSLVFLDLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDAVMSYLNAS

SEQ ID NO: 58 - DNA, 1599 bp, Artificial Sequence
  source: /note=“Description of Artificial Sequence Synthetic polynucleotide”
  source: /note=“DNA sequence encoding Destabilized IL-12 #3 (DSIL-12 #3) having AA substituted p40 and p35 polypeptides linked as a single chain fusion protein by a thrombin target site linker”
atgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctcacctgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgatatcctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccctggtgcccagaggcagctctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatgccgtgatgagctatctgaatgcttcctaa

SEQ ID NO: 59 - protein, 7 aa, Artificial Sequence
  source: /note=“Description of Artificial Sequence Synthetic Gly-Ser linker”
GGGGGGS

SEQ ID NO: 60 - protein, 7 aa, Artificial Sequence
  source: /note=“Description of Artificial Sequence Synthetic thrombin target site linker”
LVPRGSS

SEQ ID NO: 61 - protein, 15 aa, Artificial Sequence
  source: /note=“Description of Artificial Sequence Synthetic linker”
GGGGSGGGGSGGGGS

SEQ ID NO: 62 - protein, 8 aa, Homo sapiens
PQFRIKGG

SEQ ID NO: 63 - protein, 9 aa, Homo sapiens
KTKQLRVVN

SEQ ID NO: 64 - protein, 11 aa, Homo sapiens
FPQFRIKGGPI

SEQ ID NO: 65 - protein, 25 aa, Homo sapiens
GGGSGGGGSPQFRIKGGGGGSGGGS

SEQ ID NO: 66 - protein, 30 aa, Homo sapiens
PQFRIKGGGGSPQFRIKGGGGSPQFRIKGG

SEQ ID NO: 67 - protein, 18 aa, Homo sapiens
GGGGSFSARGHRPGGGGS

SEQ ID NO: 68 - protein, 17 aa, Homo sapiens
GGGGSLVPRGSSGGGGS

SEQ ID NO: 69 - protein, 19 aa, Homo sapiens
GGGGSKTKQLRVVNGGGGS

SEQ ID NO: 70 - protein, 18 aa, Homo sapiens
GGGGSQEPVKGPVGGGGS

SEQ ID NO: 71 - protein, 19 aa, Homo sapiens
GGGGSVEKRRNSGPGGGGS

SEQ ID NO: 72 - protein, 32 aa, Homo sapiens
GGSPQFRIKGGKTKQLRVVNVEKRRNSGPGGS

SEQ ID NO: 73 - protein, 24 aa, Homo sapiens
GGGSRRPYLKVFNPRRKLEFGGGS

SEQ ID NO: 74 - protein, 22 aa, Homo sapiens
GGGSNSGRAVTYSRSRYLGGGS

SEQ ID NO: 75 - protein, 24 aa, Homo sapiens
GGGSCPGRVVGGPQFRIKGGGGGS

SEQ ID NO: 76 - protein, 26 aa, Homo sapiens
GGGSGGGGSLVPRGSSGGGGSGGGGS

SEQ ID NO: 77 - protein, 16 aa, Homo sapiens
GGGSSKGRSLIGGGGS

SEQ ID NO: 78 - protein, 16 aa, Homo sapiens
GGGSQRYKVDYEGGGS

SEQ ID NO: 79 - protein, 16 aa, Homo sapiens
GGGSPQSRSVPPGGGS

SEQ ID NO: 80 - protein, 16 aa, Homo sapiens
GGGSKKPRCGVPGGGS

SEQ ID NO: 81 - protein, 7 aa, Homo sapiens
ASGREPI

SEQ ID NO: 82 - protein, 6 aa, Homo sapiens
GPAPTS

SEQ ID NO: 83 - protein, 8 aa, Homo sapiens
FSARGHRP

SEQ ID NO: 84 - protein, 8 aa, Homo sapiens
YIHPFHLV

SEQ ID NO: 85 - protein, 8 aa, Homo sapiens
HPFHLVIH

SEQ ID NO: 86 - protein, 8 aa, Homo sapiens
KQSRKFVP

SEQ ID NO: 87 - protein, 8 aa, Homo sapiens
PQSRSVPP

SEQ ID NO: 88 - protein, 16 aa, Homo sapiens
RRPYLKVFNPRRKLEF

SEQ ID NO: 89 - protein, 8 aa, Homo sapiens
VSPRASAS

SEQ ID NO: 90 - protein, 8 aa, Homo sapiens
SMTRVVGG

SEQ ID NO: 91 - protein, 8 aa, Homo sapiens
LDPRSFLL

SEQ ID NO: 92 - protein, 8 aa, Homo sapiens
SKGRSLIG

SEQ ID NO: 93 - protein, 8 aa, Homo sapiens
LGIRSFRN

SEQ ID NO: 94 - protein, 8 aa, Homo sapiens
HKGRSALV

SEQ ID NO: 95 - protein, 8 aa, Homo sapiens
KLQPRALA

SEQ ID NO: 96 - protein, 8 aa, Homo sapiens
NALRYAPD

SEQ ID NO: 97 - protein, 9 aa, Homo sapiens
PEAKASCYG

SEQ ID NO: 98 - protein, 8 aa, Homo sapiens
LRPRIIGG

SEQ ID NO: 99 - protein, 8 aa, Homo sapiens
QRYKVDYE

SEQ ID NO: 100 - protein, 8 aa, Homo sapiens
SRLRAYLL

SEQ ID NO: 101 - protein, 8 aa, Homo sapiens
AHPRIISA

SEQ ID NO: 102 - protein, 10 aa, Homo sapiens
PSSRRRVNSL

SEQ ID NO: 103 - protein, 8 aa, Homo sapiens
SKLRVVGG

SEQ ID NO: 104 - protein, 8 aa, Homo sapiens
VEVKSEKL

SEQ ID NO: 105 - protein, 9 aa, Homo sapiens
VEKRRNSGP

SEQ ID NO: 106 - protein, 8 aa, Homo sapiens
KKPRCGVP

SEQ ID NO: 107 - protein, 12 aa, Homo sapiens
ANVRRKRYAIQG

SEQ ID NO: 108 - protein, 8 aa, Homo sapiens
QEPVKGPV

SEQ ID NO: 109 - protein, 14 aa, Homo sapiens
NSGRAVTYSRSRYL

SEQ ID NO: 110 - protein, 8 aa, Homo sapiens
CPGRVVGG

SEQ ID NO: 111 - protein, 13 aa, Homo sapiens
FLEAIPMSIPPEV

SEQ ID NO: 112 - protein, 13 aa, Homo sapiens
LSEITAYSMPPVI

SEQ ID NO: 113 - protein, 13 aa, Homo sapiens
HLEEKAWSKYQTV

SEQ ID NO: 114 - protein, 13 aa, Homo sapiens
AVKITLLSALVET

SEQ ID NO: 115 - protein, 13 aa, Homo sapiens
TFAIKFFSAQTNR

SEQ ID NO: 116 - protein, 13 aa, Homo sapiens
GTIFTFRSARLNS

SEQ ID NO: 117 - protein, 13 aa, Homo sapiens
TTKFIVRSKDGPS

SEQ ID NO: 118 - protein, 13 aa, Homo sapiens
AGIATFCMLMPEE

SEQ ID NO: 119 - protein, 13 aa, Homo sapiens
GSEIDIRIRVPSI

SEQ ID NO: 120 - protein, 13 aa, Homo sapiens
GDSIAVKSLPMRA

SEQ ID NO: 121 - protein, 13 aa, Homo sapiens
GAVVSERSLRSWV

SEQ ID NO: 122 - protein, 13 aa, Homo sapiens
GIGFTVTSAPGHE

SEQ ID NO: 123 - protein, 13 aa, Homo sapiens
GGVMTGRTGHGGP

SEQ ID NO: 124 - protein, 13 aa, Homo sapiens
AVVGFGSSPTSTN

SEQ ID NO: 125 - protein, 13 aa, Homo sapiens
AVVVVELSSPSTN

SEQ ID NO: 126 - protein, 13 aa, Homo sapiens
AAIMMMRCARFVP

SEQ ID NO: 127 - protein, 13 aa, Homo sapiens
GSNIVEKQLPQST

SEQ ID NO: 128 - protein, 13 aa, Homo sapiens
AVVRNSRCSRMEP

SEQ ID NO: 129 - protein, 13 aa, Homo sapiens
SCFVVAECCMESG

SEQ ID NO: 130 - protein, 13 aa, Homo sapiens
AVVIAGRSLNPNR

SEQ ID NO: 131 - protein, 13 aa, Homo sapiens
TVGFMPLSTQVRF

SEQ ID NO: 132 - protein, 13 aa, Homo sapiens
AVIVSARMAPEEI

SEQ ID NO: 133 - protein, 13 aa, Homo sapiens
TAILIARSSPPWF

SEQ ID NO: 134 - protein, 13 aa, Homo sapiens
SIAMSRMSLSSFS

SEQ ID NO: 135 - protein, 13 aa, Homo sapiens
AISVARTLLVFEV

SEQ ID NO: 136 - protein, 13 aa, Homo sapiens
GMIAISRMAVLYP

SEQ ID NO: 137 - protein, 13 aa, Homo sapiens
GIHIPVIMSLAQS

SEQ ID NO: 138 - protein, 8 aa, Homo sapiens
ASARTTGS

SEQ ID NO: 139 - protein, 532 aa, Artificial Sequence; identical to SEQ ID NO: 36
  source: /note=“Description of Artificial Sequence Synthetic polypeptide”
  source: /note=“Wild-type (non-destabilized) IL-12 (WTscIL-12-S) with the p40 and p35 polypeptides linked as single chain fusion protein by a short Gly-Ser linker (no protease target site)”

SEQ ID NO: 140 - protein, 540 aa, Artificial Sequence; identical to SEQ ID NO: 34
  source: /note=“Description of Artificial Sequence Synthetic polypeptide”
  source: /note=“Wild-type (non-destabilized) IL-12 (WTscIL-12-L) with the p40 and p35 polypeptides linked as single chain fusion protein by a longer Gly-Ser linker (no protease target site)”

SEQ ID NO: 141 - protein, 532 aa, Artificial Sequence
  source: /note=“Description of Artificial Sequence Synthetic polypeptide”
  source: /note=“Destabilized IL-12 #2 (DSIL-12 #2) having AA substituted p40 and p35 polypeptides linked as a single chain fusion protein by a thrombin target site linker”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDKVMSYLNAS

SEQ ID NO: 142 - protein, 532 aa, Artificial Sequence
  source: /note=“Description of Artificial Sequence Synthetic polypeptide”
  source: /note=“Destabilized IL-12 #4 (DSIL-12 #4) having p40 and AA substituted p35 polypeptides linked as a single chain fusion protein by a thrombin target site linker”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDKVMSYLNAS

SEQ ID NO: 143 - protein, 532 aa, Artificial Sequence
  source: /note=“Description of Artificial Sequence Synthetic polypeptide”
  source: /note=“Destabilized IL-12 #5 (DSIL-12 #5) having AA substituted p40 and p35 polypeptides linked as a single chain fusion protein by a thrombin target site linker”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 144 - protein, 532 aa, Artificial Sequence
  source: /note=“Description of Artificial Sequence Synthetic polypeptide”
  source: /note=“Destabilized IL-12 #1 (DSIL-12 #1) having AA substituted p40 and p35 polypeptides linked as a single chain fusion protein by a thrombin target site linker”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDAVMSYLNAS

SEQ ID NO: 145 - protein, 532 aa, Artificial Sequence
  source: /note=“Description of Artificial Sequence Synthetic polypeptide”
  source: /note=“Destabilized IL-12 #3 (DSIL-12 #3) having AA substituted p40 and p35 polypeptides linked as a single chain fusion protein by a thrombin target site linker”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDAVMSYLNAS

SEQ ID NO: 146 - protein, 533 aa, Artificial Sequence
  source: /note=“Description of Artificial Sequence Synthetic polypeptide”
  source: /note=“Destabilized IL-12 #6 (DSIL-12 #6) having AA substituted p40 and p35 polypeptides linked as a single chain fusion protein by a plasmin target site linker”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSPQFRIKGGRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 147 - protein, 534 aa, Artificial Sequence
  source: /note=“Description of Artificial Sequence Synthetic polypeptide”
  source: /note=“Destabilized IL-12 #7 (DSIL-12 #7) having AA substituted p40 and p35 polypeptides linked as a single chain fusion protein by a urokinase plasminogen activator (uPA) target site linker”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSKTKQLRVVNRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 148 - protein, 533 aa, Artificial Sequence
  source: /note=“Description of Artificial Sequence Synthetic polypeptide”
  source: /note=“Destabilized IL-12 #8 (DSIL-12 #8) having AA substituted p40 and p35 polypeptides linked as a single chain fusion protein by a plasmin target site linker and a plasmin target within the heparin-binding region of p40”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVFPQFRIKGGPIDRVFTDKTSATVICRKNASISVRAQDRYYSSSWSEWASVPCSPQFRIKGGRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 149 - protein, 532 aa, Artificial Sequence
  source: /note=“Description of Artificial Sequence Synthetic polypeptide”
  source: /note=“Destabilized IL-12 #9 (DSIL-12 #9) having AA substituted p40 and p35 polypeptides linked as a single chain fusion protein by a short Gly-Ser linker (no protease target site)”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 150 - protein, 540 aa, Artificial Sequence
  source: /note=“Description of Artificial Sequence Synthetic polypeptide”
  source: /note=“Destabilized IL-12 #10 (DSIL-12 #10) having AA substituted p40 and p35 polypeptides linked as a single chain fusion protein by a long Gly-Ser linker (no protease target site)”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 151 - protein, 550 aa, Artificial Sequence
  source: /note=“Description of Artificial Sequence Synthetic polypeptide”
  source: /note=“Destabilized IL-12 #11 (DSIL-12 #11) having AA substituted p40 and p35 polypeptides linked as a single chain fusion protein by a long linker containing a plasmin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGGSPQFRIKGGGGGSGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 152 - protein, 555 aa, Artificial Sequence
  source: /note=“Description of Artificial Sequence Synthetic polypeptide”
  source: /note=“Destabilized IL-12 #12 (DSIL-12 #12) having AA substituted p40 and p35 polypeptides linked as a single chain fusion protein by a linker containing multiple plasmin sequences”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSPQFRIKGGGGSPQFRIKGGGGSPQFRIKGGRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 153 - protein, 543 aa, Artificial Sequence
  source: /note=“Description of Artificial Sequence Synthetic polypeptide”
  source: /note=“Destabilized IL-12 #13 (DSIL-12 #13) having AA substituted p40 and p35 polypeptides linked as a single chain fusion protein by a long linker containing a thrombin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSFSARGHRPGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 154 - protein, 542 aa, Artificial Sequence
  source: /note=“Description of Artificial Sequence Synthetic polypeptide”
  source: /note=“Destabilized IL-12 #14 (DSIL-12 #14) having AA substituted p40 and p35 polypeptides linked as a single chain fusion protein by a long linker containing a thrombin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSLVPRGSSGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 155 - protein, 544 aa, Artificial Sequence
  source: /note=“Description of Artificial Sequence Synthetic polypeptide”
  source: /note=“Destabilized IL-12 #15 (DSIL-12 #15) having AA substituted p40 and p35 polypeptides linked as a single chain fusion protein by a long linker containing a urokinase plasminogen activator (uPA) target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSKTKQLRVVNGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 156 - protein, 543 aa, Artificial Sequence
  source: /note=“Description of Artificial Sequence Synthetic polypeptide”
  source: /note=“Destabilized IL-12 #16 (DSIL-12 #16) having AA substituted p40 and p35 polypeptides linked as a single chain fusion protein by a long linker containing a neutrophil elastase target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSQEPVKGPVGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 157 - protein, 544 aa, Artificial Sequence
  source: /note=“Description of Artificial Sequence Synthetic polypeptide”
  source: /note=“Destabilized IL-12 #17 (DSIL-12 #17) having AA substituted p40 and p35 polypeptides linked as a single chain fusion protein by a long linker containing a plasma kallikrein target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSVEKRRNSGPGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 158 - protein, 557 aa, Artificial Sequence
  source: /note=“Description of Artificial Sequence Synthetic polypeptide”
  source: /note=“Destabilized IL-12 #18 (DSIL-12 #18) having AA subst. p40 and p35 polypeptides linked as a single chain fusion protein by linker containing plasmin, urokinase plasminogen activator (uPA) and plasma kallikrein target sequences”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSPQFRIKGGKTKQLRVVNVEKRRNSGPGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 159 - protein, 549 aa, Artificial Sequence
  source: /note=“Description of Artificial Sequence Synthetic polypeptide”
  source: /note=“Destabilized IL-12 #19 (DSIL-12 #19) having AA substituted p40 and p35 polypeptides linked as a single chain fusion protein by a long linker containing several plasmin target sequences”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSRRPYLKVFNPRRKLEFGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 160 - protein, 547 aa, Artificial Sequence
  source: /note=“Description of Artificial Sequence Synthetic polypeptide”
  source: /note=“Destabilized IL-12 #20 (DSIL-12 #20) having AA substituted p40 and p35 polypeptides linked as a single chain fusion protein by a long linker containing a plasmin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSNSGRAVTYSRSRYLGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 161 - protein, 549 aa, Artificial Sequence
  source: /note=“Description of Artificial Sequence Synthetic polypeptide”
  source: /note=“Destabilized IL-12 #21 (DSIL-12 #21) having AA substituted p40 and p35 polypeptides linked as a single chain fusion protein by a linker containing a tissue plasminogen activator target sequence and a plasmin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSCPGRVVGGPQFRIKGGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 162 - protein, 551 aa, Artificial Sequence
  source: /note=“Description of Artificial Sequence Synthetic polypeptide”
  source: /note=“Destabilized IL-12 #22 (DSIL-12 #22) having AA substituted p40 and p35 polypeptides linked as a single chain fusion protein by a long linker containing a thrombin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGGSLVPRGSSGGGGSGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 163 - protein, 541 aa, Artificial Sequence
  source: /note=“Description of Artificial Sequence Synthetic polypeptide”
  source: /note=“Destabilized IL-12 #23 (DSIL-12 #23) having AA substituted p40 and p35 polypeptides linked as a single chain fusion protein by a long linker containing a plasmin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSSKGRSLIGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 164 - protein, 541 aa, Artificial Sequence
  source: /note=“Description of Artificial Sequence Synthetic polypeptide”
  source: /note=“Destabilized IL-12 #24 (DSIL-12 #24) having AA substituted p40 and p35 polypeptides linked as a single chain fusion protein by a long linker containing a plasmin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSQRYKVDYEGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 165 - protein, 541 aa, Artificial Sequence
  source: /note=“Description of Artificial Sequence Synthetic polypeptide”
  source: /note=“Destabilized IL-12 #25 (DSIL-12 #25) having AA substituted p40 and p35 polypeptides linked as a single chain fusion protein by a long linker containing a plasmin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSPQSRSVPPGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 166 - protein, 541 aa, Artificial Sequence
  source: /note=“Description of Artificial Sequence Synthetic polypeptide”
  source: /note=“Destabilized IL-12 #26 (DSIL-12 #26) having AA substituted p40 and p35 polypeptides linked as a single chain fusion protein by a long linker containing a plasmin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSKKPRCGVPGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 167 - protein, 533 aa, Artificial Sequence
  source: /note=“Description of Artificial Sequence Synthetic polypeptide”
  source: /note=“Destabilized IL-12 #27 (DSIL-12 #27) having AA substituted p40 and p35 polypeptides linked as a single chain fusion protein by a plasmin target site linker”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGASGREPIDRVFTDKTSATVICRKNASISVRAQDRYYSSSWSEWASVPCSPQFRIKGGRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 168 - protein, 532 aa, Artificial Sequence
  source: /note=“Description of Artificial Sequence Synthetic polypeptide”
  source: /note=“Single-chain (non-destabilized) IL-12 #28 (SCIL-12 #28) having p40 and p35 polypeptides linked as single chain fusion protein by a thrombin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSLVPRGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 169 - protein, 542 aa, Artificial Sequence
  source: /note=“Description of Artificial Sequence Synthetic polypeptide”
  source: /note=“Single-chain (non-destabilized) IL-12 #29 (SCIL-12 #29) having p40 and p35 polypeptides linked as single chain fusion protein by a long linker containing a thrombin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSLVPRGSSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 170 - protein, 551 aa, Artificial Sequence
  source: /note=“Description of Artificial Sequence Synthetic polypeptide”
  source: /note=“Single-chain (non-destabilized) IL-12 #30 (SCIL-12 #30) having p40 and p35 polypeptides linked as single chain fusion protein by an extended long linker containing a thrombin target sequence”
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGGSLVPRGSSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

(19) United States
(12) Patent Application Publication
(10) Pub. No.: US 2019/0062394 A1
(43) Pub. Date: Feb. 28, 2019

(54) Improved Therapeutic Control of Proteolytically Sensitive, Destabilized Forms of Interleukin-12

(71) Applicant: Intrexon Corporation, Blacksburg, VA (US)

(72) Inventors: Ramya YARLAGADDA, Blacksburg, VA (US); Charles REED, Blacksburg, VA (US); Charles Andrew (Marshall) STEWART, Blacksburg, VA (US); Peter EMTAGE, Blacksburg, VA (US); Rutul SHAH, Blacksburg, VA (US); Timothy CHAN, Blacksburg, VA (US)

(73) Assignee: Intrexon Corporation, Blacksburg, VA (US)

(21) Appl. No.: 15/767,298

(22) PCT Filed: Oct. 10, 2016
(86) PCT No.: PCT/US2016/056281

§ 371 Date: Apr. 10, 2018

Related U.S. Application Data
(60) Provisional application No. 62/239,871, filed on Oct. 10, 2015.

(51) Int. Cl. C07K 14/54; A61P 35/00

(52) U.S. Cl. CPC C07K 14/5434; A61P 35/00; C07K 2319/03; C07K 2319/02; C07K 2319/50

(57) ABSTRACT

The present invention relates to modified forms of IL-12. These modified forms of IL-12 may be engineered to have a shortened in vivo half-life compared and/or enhanced localization of biological effects compared to that of corresponding non-modified form of IL-12. Short half-life and membrane bound forms of IL-12 may provide greater therapeutic control for in vivo therapeutic delivery, in particular when used in combination with ligand inducible delivery of IL-12. Modified forms of IL-12 engineered to have shortened in vivo half-life and/or enhanced localization of biological effects include heterodimeric p35/p40, single chain and membrane bound forms of IL-12 wherein a naturally occurring IL-12 amino acid sequence is genetically modified to destabilize IL-12 tertiary structure/polypeptide folding and enhance susceptibility of the IL-12 molecule to in vivo proteolytic degradation.

### REFERENCE TO SEQUENCE LISTING

The instant application contains a Sequence Listing which has been submitted electronically in ASCII format and is hereby incorporated by reference in its entirety. Said ASCII copy, created on Oct. 10, 2016, is named 0100-0023WO1_SL.txt and is 358,758 bytes in size.

### FIELD OF THE INVENTION

The present invention provides novel nucleic acids encoding modified forms of interleukin-12 (IL-12) for enhanced in vivo therapeutic control and dose regulation. The present invention also provides vectors comprising such nucleic acids, polypeptides encoded by such nucleic acids, and for use of such compositions in therapeutic applications in which IL-12 is beneficial.

### BACKGROUND OF THE INVENTION

Human IL-12 p70 (i.e., dimer of p35 and p40) has a reported in vivo half-life of 5-19 hours which, when administered as a therapeutic compound, can result in significant systemic toxicity. See e.g., Car et al. “The Toxicology of Interleukin-12: A Review” Toxicologic Path. 27:1, 58-63 (1999); Robertson et al. “Immunological Effects of Interleukin 12 Administered by Bolus Intravenous Injection to Patients with Cancer” Clin. Cancer Res. 5:9-16 (1999); Atkins et al. “Phase I Evaluation of Intravenous Recombinant Human Interleukin 12 in Patients with Advance Malignancies” Clin. Cancer Res. 3:409-417 (1997).

While ligand inducible control of IL-12 gene expression can regulate IL-12 production in a dose dependent fashion, the time from cessation (stopping administration) of ligand dosing to cessation of protein synthesis and IL-12 clearance (“decay”) may be insufficient to prevent toxic accumulation of IL-12 in plasma. As such, strategies for example, of engineering tumor lymphocytes with spatial and temporal control of traditional forms of IL-12 may be insufficient to optimally control IL-12 systemic toxicity.

Therefore, there remains a need in the art for improved therapeutic control of in vivo delivered forms of IL-12, for example as vaccine adjuvants and in the treatment of infections and cancer.

### Heterodimeric IL-12

Interleukin-12 (IL-12) is a heterodimeric molecule composed of an alpha chain (the p35 subunit) and a beta chain (the p40 subunit) covalently linked by a disulfide bridge to form the biologically active 70 kDa dimer. Biologically, IL-12 is an inflammatory cytokine that is produced in response to infection by a variety of cells of the immune system, including phagocytic cells, B cells and activated dendritic cells (Colombo and Trinchieri (2002), Cytokine & Growth Factor Reviews, 13: 155-168 and Hamza et al., “Interleukin-12 a Key Immunoregulatory Cytokine in Infection Applications” Int. J. Mol. Sci. 11; 789-806 (2010). IL-12 plays an essential role in mediating the interaction of the innate and adaptive arms of the immune system, acting on T-cells and natural killer (NK) cells, enhancing the proliferation and activity of cytotoxic lymphocytes and the production of other inflammatory cytokines, especially interferon-gamma (IFN-gamma).

IL-12 has been tested in human clinical trials as an immunotherapeutic agent for the treatment of a wide variety of cancers (Atkins et al. (1997), Clin. Cancer Res., 3: 409-17; Gollob et al. (2000), Clin. Cancer Res., 6: 1678-92; Hurteau et al. (2001), Gynecol. Oncol., 82: 7-10; and Youssoufian, et al. (2013) Surgical Oncology Clinics of North America, 22(4): 885-901), including renal, colon, and ovarian cancer, melanoma and T-cell lymphoma, and as an adjuvant for cancer vaccines (Lee et al. (2001), J. Clin. Oncol. 19: 3836-47). However, IL-12 is toxic when administered systemically as a recombinant protein. Trinchieri, Adv. Immunol. 1998; 70:83-243. In order to maximize the anti-tumoral effect of IL-12 while minimizing its systemic toxicity, IL-12 gene therapy approaches have been proposed to allow production of the cytokine at the tumor site, thereby achieving high local levels of IL-12 with low serum concentration. Qian et al., Cell Research (2006) 16: 182-188; US Patent Publication 20130195800.

### Single Chain IL-12

Since IL-12 is a heterodimeric molecule composed of an alpha chain (the p35 subunit) and a beta chain (the p40 subunit), the simultaneous expression of the two subunits is necessary for the production of the biologically active heterodimer. Recombinant IL-12 expression has been achieved using bicistronic vectors containing the p40 and p35 subunits separated by an IRES (internal ribosome entry site) sequence to allow independent expression of both subunits from a single vector. However, use of IRES sequences can impair protein expression. Mizuguchi et al., Mol Ther (2000); 1: 376-382. Moreover, unequal expression of the p40 and p35 subunits can lead to the formation of homodimeric proteins (e.g., p40-p40) which can have inhibitory effects on IL-12 signaling. Gillessen et al. Eur. J. Immunol. 25(1):200-6 (1995).

As an alternative to bicistronic expression of the IL-12 subunits, functional single chain IL-12 fusion proteins have been produced by joining the p40 and p35 subunits with (Gly4Ser)3 (SEQ ID NO: 61) or Gly6Ser (SEQ ID NO: 59) linkers. Lieschke et al., (1997), Nature Biotechnology 15, 35-40; Lode et al., (1998), PNAS 95, 2475-2480. (These forms of p40-linker-p35 or p35-linker-p40 IL-12 configurations may be referred to herein as “traditional single chain IL-12 (scIL-12)”.) Notably, however, long linker sequences may interfere with the ability to construct viral vectors for gene therapy, and may increase the likelihood of inducing immunogenic responses (e.g., by generating anti-single chain IL-12 antibodies).

### BRIEF SUMMARY OF THE INVENTION

The present invention relates to modified forms of IL-12. These modified forms of IL-12 are engineered to have a shortened in vivo half-life and/or enhanced localization of biological effects compared to that of corresponding non-modified forms of IL-12. Short half-life and membrane bound forms of IL-12 provide greater therapeutic control for in vivo therapeutic delivery, in particular when used in combination with ligand inducible expression and delivery of IL-12. Modified forms of IL-12 engineered to have shortened in vivo half-life and/or enhanced localization of biological effects include heterodimeric p35/p40, single chain and membrane bound forms of IL-12 wherein naturally occurring IL-12 amino acid sequences are genetically modified with one or more proteolytic target sequences and one or more amino acid substitution which destabilize inter- or intra-chain protein-protein (e.g., amino acid-amino acid) interactions thereby enhancing susceptibility of the IL-12 polypeptides to in vivo proteolytic degradation and/or decreasing one or more IL-12 biological activities. See e.g., Examples herein. Previous examples of IL-12 comprising proteolytic target sequences, but lacking further destabilization modifications, are described in PCT/US2015/051246 (WO 2016/048903), filed 21 Sep. 2015, which is hereby incorporated by reference in its entirety.

Modified forms of IL-12 include dimeric IL-12 polypeptides (heterodimers of IL-12 p35/p40 polypeptides), various forms of single-chain interleukin-12 fusion proteins (scIL-12), and membrane-bound forms of heterodimeric or single-chain IL-12 polypeptides (mbIL-12) which have been engineered to comprise proteolytic and destabilizing amino acid sequences. Modified forms of IL-12 comprising non-naturally occurring proteolytic and destabilizing sequences function to shorten in vivo half-life and/or biological activity. (In this context, “non-naturally occurring proteolytic and destabilizing sequences” means proteolytic amino acid sites or sequences not found in wild-type IL-12 polypeptide sequences or encoded by naturally occurring IL-12 genes/polynucleotides.) In certain embodiments, IL-12 polypeptides of the invention (i.e., “modified” IL-12 polypeptides) are engineered to comprise amino acid sequences which are preferentially targeted by any one or more of matrix metalloproteinase-2 (MMP-2), plasmin, thrombin, urokinase-type plasminogen activator (uPA), and/or carboxypeptidases (e.g., acting in concert with endoproteinases or enteropeptidases).

Modified forms of destabilized IL-12 as described herein are engineered to have plasma proteinase cleavage sites. Multiple locations exist on IL-12 to engineer proteinase cleavage sites. Cleavage sites are engineered into the IL-12 p35 domain, the IL-12 p40 domain, or both the IL-12 p35 and p40 domain; in any of heterodimeric IL-12, single-chain IL-12 (scIL-12), or membrane bound forms of IL-12 (mbIL-12). For single chain and membrane bound forms of destabilized IL-12, in addition to or instead of the p35 and p40 subunits, proteinase cleavage sites are engineered into the linker or membrane-anchoring sequences used to generate the IL-12 fusion protein. Modified forms of destabilized IL-12 are engineered to be rapidly cleared from the in vivo blood plasma.

The present invention also comprises destabilized single chain IL-12 (scIL-12) polypeptides wherein the length of linker sequences, if any, is minimized by inserting IL-12 p35 polypeptide sequences within an IL-12 p40 polypeptide sequence while retaining at least one IL-12 biological activity. (These forms of p40N-p35-p40C IL-12 configurations may be referred to herein as “topologically manipulated single chain IL-12 (scIL-12)” or variations thereon such as “topo scIL-12” or simply “topo IL-12”. See, PCT/US2014/070695 (WO 2015/095249), filed 17 Dec. 2014, which is hereby incorporated by reference in its entirety.) In one embodiment, such destabilized scIL-12 polypeptides are modified to comprise amino acid substitutions and proteolytic amino acid sequences, thereby rendering the biologically active composition susceptible to reduced in vivo (plasma) half-life and/or reduced biological activity.

The present invention further comprises modified topologically manipulated (“topo”) destabilized scIL-12 polypeptides comprising, from N- to C-terminus:

(i) a first IL-12 p40 domain (p40N),

(ii) an optional first peptide linker,

(iii) an IL-12 p35 domain,

(iv) an optional second peptide linker, and

(v) a second IL-12 p40 domain (p40C).

See e.g., PCT/US2014/70695 (WO2015/095249) which is hereby incorporated by reference herein in its entirety.

In one embodiment, topologically manipulated (“topo”) destabilized scIL-12 polypeptides are modified to comprise amino acid substitutions and proteolytic amino acid sequences, thereby rendering the biologically active composition susceptible to reduced in vivo (plasma) half-life and/or reduced biological activity.

In certain embodiments, destabilized IL-12, scIL-12 and mbIL-12 polypeptides of the invention retain at least one biological activity of a reference IL-12, scIL-12 and mbIL-12, respectively.

The invention includes destabilized modified IL-12, scIL-12 and mbIL-12 polynucleotides encoding IL-12, scIL-12 and mbIL-12 polypeptides as described herein, respectively, and to vectors comprising said IL-12, scIL-12 and mbIL-12 polynucleotides, respectively.

The invention includes destabilized modified variant IL-12 and scIL-12 polypeptides and polynucleotides comprising at least 80%, 85%, 90%, 95%, 97%, 98%, or 99% identity to a reference scIL-12 polypeptide or polynucleotide.

The invention includes modified cells or non-human organisms transformed, transfected or otherwise genetically altered to contain and/or express destabilized modified IL-12, scIL-12 and mbIL-12 polynucleotides or vectors as described herein.

The invention includes pharmaceutical and diagnostic compositions comprising as an active agent destabilized modified IL-12, scIL-12 and mbIL-12 polypeptides, polynucleotides, vectors, or cells as described herein.

The invention includes methods of using destabilized modified IL-12, scIL-12 and mbIL-12 polypeptides, polynucleotides, vectors and cells of the invention for enhancing immune system function, for example, but not limited to, use as vaccine adjuvants and in the treatment of infections, cancer and immune system disorders or pathologies.

### DESCRIPTION OF THE FIGURES

FIG. 1 provides a schematic depiction of approaches for improved therapeutic control of IL-12. Upper portion of figure depicts expression and secretion from a modified cell generated to express IL-12 genetically engineered to comprise non-naturally occurring proteolytic sites, thereby resulting in rapid degradation/breakdown (proteolysis) and clearance. Lower portion of the figure depicts highly localized (concentrated) biological effects/activities mediated by membrane bound IL-12 (Objects not to scale). As described further herein, membrane bound and protease sensitivity features are combined (engineered) into a single IL-12 compound (single chain or heterodimeric forms).

FIG. 2 provides schematic diagrams showing the p40-p35 single chain configuration (FIG. 1A), the p35-p40 single chain configuration (FIG. 1B), and a p40N-p35-p40C insert configuration (FIG. 1C). Construction and characterization of these designs are discussed in detail elsewhere herein.

FIG. 3 shows expression levels of human scIL-12 designs as determined by p70 ELISA (see Example 2).

FIG. 4 shows scIL-12 stimulated IFN-gamma production as measured by ELISA (see Example 3).

FIG. 5 shows highly exposed loops on IL-12 which targeted for engineering in proteinase cleavage sites.

FIG. 6 schematically depicts a membrane bound form of single chain IL-12.

FIG. 7 depicts non-limiting examples of membrane bound IL-12 polypeptide constructs. For example, first row depicts a membrane-tethered single chain polypeptide in which IL-12 is anchored to the cell membrane via fusion to a transmembrane (TMD) and cytoplasmic domain (CD) of CD80. Other TMDs and CDs may be substituted in place of CD80. Second row depicts a membrane-tethered single chain polypeptide in which IL-12 is anchored to the cell membrane via a decay accelerating factor (DAF) glycosylphosphatidylinositol membrane anchoring moiety. Third row depicts a membrane-tethered single chain polypeptide in which IL-12 is anchored to the cell membrane via a CD59 GPI membrane anchoring moiety.

Glycosylphosphatidylinositol (GPI) anchor is a glycolipid structure that is post-translationally linked to the C-terminus of some eukaryotic proteins. It is composed of a phosphatidylinositol group linked through a carbohydrate-containing linker (glucosamine and mannose glycosidically bound to the inositol residue) and via an ethanolamine phosphate (EtNP) bridge to the C-terminal amino acid of a mature protein. The two fatty acids within the hydrophobic phosphatidyl-inositol group function to anchor the linked protein to the extracellular surface of the cell membrane.

FIG. 8 is a schematic depiction of critical amino acid residues and intrachain polypeptide interactions in a single-chain IL-12 fusion polypeptide.

FIG. 9 includes examples of IL-12 Wild-Type and Destabilized Polypeptide Components. FIG. 9A is wild-type IL-12 p40 polypeptide (SEQ ID NO:43). The p40 predicted 22 AA signal peptide sequence is noted with the wavey underline. The p40 wild-type Cys-177 and Asp-290 AA residues are noted with the double-underline (form bonds with Cys-74 and Arg-189 respectively in p35 polypeptide). FIG. 9B includes the sequence for destabilized #2 IL-12 p40 polypeptide (SEQ ID NO:44). The p40 predicted 22 AA signal peptide sequence is noted with wavey underline. p40 Destabilized #2 equals p40 with Cys-177 AA substituted with Ser-177 (a.k.a., C177S). p40 Destabilized #2 equals p40 with Asp-290 AA residues double-underlined (forms ionic bond with p35 Arg-189).

FIG. 9C is the Gly-Ser Linker (No Thrombin site) (SEQ ID NO:59). FIG. 9D is the Destabilized #2 Thrombin Linker (SEQ ID NO:60). FIG. 9E is the wild-type IL-12 p35 polypeptide (SEQ ID NO:45). The p35 wild-type Cys-74 AA and Arg-189 AA residues are double-underlined (these AA residues form bonds with Cys-177 and Asp-290 respectively in p40 polypeptide).

FIG. 9F is the destabilized #2 IL-12 p35 polypeptide (SEQ ID NO:46). The figure shows (i) p35 Cys-74 substituted with Ser-74 (C74S) and (ii) p35 Arg-189 substituted with Lys-189 (R189K) (double underlined). FIG. 9G represents the wild-type (non-destabilized) IL-12 with the p40 and p35 polypeptides linked as single chain fusion protein by a Gly-Ser linker (no thrombin target site) (SEQ ID NO:47).

FIG. 9H is the polypeptide sequence of destabilized IL-12 #2 (DSIL-12 #2) having AA substituted p40 and p35 polypeptides linked as a single chain fusion protein by a thrombin target site linker (SEQ ID NO:49). The figure shows (i) destabilized #2 p40 predicted 22 AA signal peptide sequence (wavey underline), (ii) destabilized #2 p40 polypeptide having Cys-177 substituted with Ser-177 (double-underline) (C177S), (iii) destabilized #2 having “LVPRGSS” thrombin target sequence (SEQ ID NO: 60) (dotted underline), (iv) destabilized #2 p35 polypeptide having Cys-74 substituted with Ser-74 (double-underline)(C74S), and (v) destabilized #2 p35 polypeptide having Arg-189 substituted with Lys-189 (double-underline)(R189K)

FIG. 10 is the polypeptide sequence of destabilized IL-12 #4 (DSIL-12 #4) having p40 and AA substituted p35 polypeptides linked as a single chain fusion protein by a thrombin target site linker (SEQ ID NO:51). The figure shows (i) destabilized #4 p40 predicted 22 AA signal peptide sequence (wavey underline), (ii) destabilized #4 having “LVPRGSS” thrombin target sequence (SEQ ID NO: 60) (dotted underline), and (iii) destabilized #4 p35 polypeptide having Arg-189 substituted with Lys-189 (double-underline)(R189K).

FIG. 11 is the polypeptide sequence of destabilized IL-12 #5 (DSIL-12 #5) having AA substituted p40 and p35 polypeptides linked as a single chain fusion protein by a thrombin target site linker (SEQ ID NO:53). The figure shows (i) destabilized #5 p40 predicted 22 AA signal peptide sequence (wavey underline), (ii) destabilized #5 p40 polypeptide having Cys-177 substituted with Ser-177 (double-underline)(C177S), (iii) destabilized #5 having “LVPRGSS” thrombin target sequence (SEQ ID NO: 60) (dotted underline), and (iv) destabilized #5 p35 polypeptide having Cys-74 substituted with Ser-74 (double-underline)(C74S).

FIG. 12 is the polypeptide sequence of destabilized IL-12 #1 (DSIL-12 #1) having AA substituted p40 and p35 polypeptides linked as a single chain fusion protein by a thrombin target site linker (SEQ ID NO:55). The figure shows (i) destabilized #1 p40 predicted 22 AA signal peptide sequence (wavey underline), (ii) destabilized #1 p40 polypeptide having Cys-177 substituted with Ser-177 (double-underline) (C177S), (iii) destabilized #1 having “LVPRGSS” thrombin target sequence (SEQ ID NO: 60) (dotted underline), (iv) destabilized #1 p35 polypeptide having Cys-74 substituted with Ser-74 (double-underline) (C74S), (v) destabilized #1 p35 polypeptide having Arg-189 substituted with Ala-189 (double-underline)(R189A).

FIG. 13 is the polypeptide sequence of destabilized IL-12 #3 (DSIL-12 #3) having AA substituted p40 and p35 polypeptides linked as a single chain fusion protein by a thrombin target site linker (SEQ ID NO:57). The figure shows (i) destabilized #3 p40 predicted 22 AA signal peptide sequence (wavey underline), (ii) destabilized #3 having “LVPRGSS” thrombin target sequence (SEQ ID NO: 60) (dotted underline), and (iii) destabilized #3 p35 polypeptide having Arg-189 substituted with Ala-189 (double-underline)(R189A).

FIG. 14 is the Urokinase Plasminogen Activator (uPA) Proteolytic Linker Sequence (SEQ ID NO:63). See, e.g., Turkmen B, et al., “Mutational analysis of the genes encoding urokinase-type plasminogen activator (uPA) and its inhibitor PAI-1 in advanced ovarian cancer”, Electrophoresis (1997). PMID: 9194591.

FIG. 15 is a plasmin-sensitive proteolytic linker (SEQ ID NO:62).

FIG. 16A: Wild-type (non-destabilized) IL-12 (WTscIL-12-S) with the p40 and p35 polypeptides linked as single chain fusion protein by a short Gly-Ser linker (no protease target site) (SEQ ID NO: 139). The figure shows (i) wild-type p40 predicted 22 AA signal peptide sequence (wave-underline), (ii) wild-type p40 polypeptide having Cys-177 (double-underline), (iii) a short (7 AA) non-digestible Gly-Ser linker “GGGGGGS” (SEQ ID NO: 59) (dotted underline), (iv) wild-type p35 polypeptide having Cys-74 (double-underline) and Arg-189 (double-underline)

FIG. 16B: Wild-type (non-destabilized) IL-12 (WTscIL-12-L) with the p40 and p35 polypeptides linked as single chain fusion protein by a longer Gly-Ser linker (no protease target site) (SEQ ID NO: 140). The figure shows (i) wild-type p40 predicted 22 AA signal peptide sequence (wave-underline), (ii) wild-type p40 polypeptide having Cys-177 (double-underline), (iii) a longer (15 AA) non-digestible Gly-Ser linker “GGGGSGGGGSGGGGS” (SEQ ID NO: 61) (dotted underline), (iv) wild-type p35 polypeptide having Cys-74 (double-underline) and Arg-189 (double-underline).

FIG. 16C: Destabilized IL-12 #2 (DSIL-12 #2) having AA substituted p40 and p35 polypeptides linked as a single chain fusion protein by a thrombin target site linker (SEQ ID NO:141). The figure shows (i) destabilized #2 p40 predicted 22 AA signal peptide sequence (wave-underline), (ii) destabilized #2 p40 polypeptide having Cys-177 substituted with Ser-177 (double-underline)(C177S), (iii) destabilized #2 linker “LVPRGSS” containing a thrombin target sequence (SEQ ID NO: 60) (dotted underline), and (iv) destabilized #2 p35 polypeptide having Cys-74 substituted with Ser-74 (double-underline)(C74S).

FIG. 16D: Destabilized IL-12 #4 (DSIL-12 #4) having p40 and AA substituted p35 polypeptides linked as a single chain fusion protein by a thrombin target site linker (SEQ ID NO:142). The figure shows (i) destabilized #4 p40 predicted 22 AA signal peptide sequence (wave-underline), (ii) destabilized #4 linker “LVPRGSS” containing a thrombin target sequence (SEQ ID NO: 60) (dotted underline), and (iii) destabilized #4 p35 polypeptide having Arg-189 substituted with Lys-189 (double-underline)(R189K).

FIG. 16E: Destabilized IL-12 #5 (DSIL-12 #5) having AA substituted p40 and p35 polypeptides linked as a single chain fusion protein by a thrombin target site linker (SEQ ID NO:143). The figure shows (i)—Destabilized #5 p40 predicted 22 AA signal peptide sequence (wave-underline), (ii) destabilized #5 p40 polypeptide having Cys-177 substituted with Ser-177 (double-underline)(C177S), (iii) destabilized #5 linker “LVPRGSS” containing a thrombin target sequence (SEQ ID NO: 60) (dotted underline), and (iv) destabilized #5 p35 polypeptide having Cys-74 substituted with Ser-74 (double-underline)(C74S).

FIG. 16F: Destabilized IL-12 #1 (DSIL-12 #1) having AA substituted p40 and p35 polypeptides linked as a single chain fusion protein by a thrombin target site linker (SEQ ID NO:144). The figure shows (i) destabilized #1 p40 predicted 22 AA signal peptide sequence (wave-underline), (ii) destabilized #1 p40 polypeptide having Cys-177 substituted with Ser-177 (double-underline) (C177S), (iii) destabilized #1 linker “LVPRGSS” containing a thrombin target sequence (SEQ ID NO: 60) (dotted underline), (iv) destabilized #1 p35 polypeptide having Cys-74 substituted with Ser-74 (double-underline) (C74S), (v) destabilized #1 p35 polypeptide having Arg-189 substituted with Ala-189 (double-underline)(R189A).

FIG. 16G: Destabilized IL-12 #3 (DSIL-12 #3) having AA substituted p40 and p35 polypeptides linked as a single chain fusion protein by a thrombin target site linker (SEQ ID NO:145). The figure shows (i) destabilized #3 p40 predicted 22 AA signal peptide sequence (wave-underline), (ii) destabilized #3 linker “LVPRGSS” containing a thrombin target sequence (SEQ ID NO: 60) (dotted underline), and (iii) destabilized #3 p35 polypeptide having Arg-189 substituted with Ala-189 (double-underline)(R189A).

FIG. 16H: Destabilized IL-12 #6 (DSIL-12 #6) having AA substituted p40 and p35 polypeptides linked as a single chain fusion protein by a plasmin target site linker (SEQ ID NO:146). The figure shows (i) destabilized #6 p40 predicted 22 AA signal peptide sequence (wave-underline), (ii) destabilized #6 p40 polypeptide having Cys-177 substituted with Ser-177 (double-underline)(C177S), (iii) destabilized #6 linker “PQFRIKGG” containing a plasmin target sequence (SEQ ID NO: 62) (dotted underline), and (iv) destabilized #6 p35 polypeptide having Cys-74 substituted with Ser-74 (double-underline)(C74S).

FIG. 16I: Destabilized IL-12 #7 (DSIL-12 #7) having AA substituted p40 and p35 polypeptides linked as a single chain fusion protein by a urokinase plasminogen activator (uPA) target site linker (SEQ ID NO:147). The figure shows (i)—Destabilized #7 p40 predicted 22 AA signal peptide sequence (wave-underline), (ii) destabilized #7 p40 polypeptide having Cys-177 substituted with Ser-177 (double-underline)(C177S), (iii) destabilized #7 linker “KTKQLRVVN” containing a urokinase plasminogen activator (uPA) target sequence (SEQ ID NO: 63) (dotted underline), and (iv) destabilized #7 p35 polypeptide having Cys-74 substituted with Ser-74 (double-underline)(C74S).

FIG. 16J: Destabilized IL-12 #8 (DSIL-12 #8) having AA substituted p40 and p35 polypeptides linked as a single chain fusion protein by a plasmin target site linker and a plasmin target within the heparin-binding region of p40 (SEQ ID NO:148). The figure shows (i) destabilized #8 p40 predicted 22 AA signal peptide sequence (wave-underline), (ii) destabilized #8 p40 polypeptide having Cys-177 substituted with Ser-177 (double-underline)(C177S) and having positions 254-264 substituted with “FPQFRIKGGPI”, a plasmin target sequence (SEQ ID NO: 64) (dashed underline), (iii) destabilized #8 linker “PQFRIKGG” containing a plasmin target sequence (SEQ ID NO: 62) (dotted underline), and (iv) destabilized #8 p35 polypeptide having Cys-74 substituted with Ser-74 (double-underline)(C74S).

FIG. 16K: Destabilized IL-12 #9 (DSIL-12 #9) having AA substituted p40 and p35 polypeptides linked as a single chain fusion protein by a short Gly-Ser linker (no protease target site) (SEQ ID NO:149). The figure shows (i)—Destabilized #9 p40 predicted 22 AA signal peptide sequence (wave-underline), (ii) destabilized #9 p40 polypeptide having Cys-177 substituted with Ser-177 (double-underline)(C177S), (iii) destabilized #9 linker “GGGGGGS” containing no protease target site (SEQ ID NO: 59) (dotted underline), and (iv) destabilized #9 p35 polypeptide having Cys-74 substituted with Ser-74 (double-underline)(C74S).

FIG. 16L: Destabilized IL-12 #10 (DSIL-12 #10) having AA substituted p40 and p35 polypeptides linked as a single chain fusion protein by a long Gly-Ser linker (no protease target site) (SEQ ID NO:150). The figure shows (i) destabilized #10 p40 predicted 22 AA signal peptide sequence (wave-underline), (ii) destabilized #10 p40 polypeptide having Cys-177 substituted with Ser-177 (double-underline)(C177S), (iii) destabilized #10 linker “GGGGSGGGGSGGGGS” containing no protease target site (SEQ ID NO: 61) (dotted underline), (iv) destabilized #10 p35 polypeptide having Cys-74 substituted with Ser-74 (double-underline)(C74S).

FIG. 16M: Destabilized IL-12 #11 (DSIL-12 #11) having AA substituted p40 and p35 polypeptides linked as a single chain fusion protein by a long linker containing a plasmin target sequence (SEQ ID NO:151). The figure shows (i) destabilized #11 p40 predicted 22 AA signal peptide sequence (wave-underline), (ii) destabilized #11 p40 polypeptide having Cys-177 substituted with Ser-177 (double-underline)(C177S), (iii) destabilized #11 linker “GGGSGGGGSPQFRIKGGGGGSGGGS” containing a plasmin target site (SEQ ID NO: 65) (dotted underline), and (iv) destabilized #11 p35 polypeptide having Cys-74 substituted with Ser-74 (double-underline)(C74S).

FIG. 16N: Destabilized IL-12 #12 (DSIL-12 #12) having AA substituted p40 and p35 polypeptides linked as a single chain fusion protein by a linker containing multiple plasmin sequences (SEQ ID NO:152). The figure shows (i) destabilized #12 p40 predicted 22 AA signal peptide sequence (wave-underline), (ii) destabilized #12 p40 polypeptide having Cys-177 substituted with Ser-177 (double-underline)(C177S), (iii) destabilized #12 linker “PQFRIKGGGGSPQFRIKGGGGSPQFRIKGG” containing 3 plasmin target sites (SEQ ID NO: 66) (dotted underline), (iv) destabilized #12 p35 polypeptide having Cys-74 substituted with Ser-74 (double-underline)(C74S).

FIG. 16O: Destabilized IL-12 #13 (DSIL-12 #13) having AA substituted p40 and p35 polypeptides linked as a single chain fusion protein by a long linker containing a thrombin target sequence (SEQ ID NO:153). The figure shows (i) destabilized #13 p40 predicted 22 AA signal peptide sequence (wave-underline), (ii) destabilized #13 p40 polypeptide having Cys-177 substituted with Ser-177 (double-underline)(C177S), (iii) destabilized #13 linker “GGGGSFSARGHRPGGGGS” containing a thrombin target sequence (SEQ ID NO: 67) (dotted underline), and (iv) destabilized #13 p35 polypeptide having Cys-74 substituted with Ser-74 (double-underline)(C74S),

FIG. 16P: Destabilized IL-12 #14 (DSIL-12 #14) having AA substituted p40 and p35 polypeptides linked as a single chain fusion protein by a long linker containing a thrombin target sequence (SEQ ID NO:154). The figure shows (i) destabilized #14 p40 predicted 22 AA signal peptide sequence (wave-underline), (ii) destabilized #14 p40 polypeptide having Cys-177 substituted with Ser-177 (double-underline)(C177S), (iii) destabilized #141inker “GGGGSLVPRGSSGGGGS” containing a thrombin target sequence (SEQ ID NO: 68) (dotted underline), and (iv) destabilized #14 p35 polypeptide having Cys-74 substituted with Ser-74 (double-underline)(C74S).

FIG. 16Q: Destabilized IL-12 #15 (DSIL-12 #15) having AA substituted p40 and p35 polypeptides linked as a single chain fusion protein by a long linker containing a urokinase plasminogen activator (uPA) target sequence (SEQ ID NO:155). The figure shows (i) destabilized #15 p40 predicted 22 AA signal peptide sequence (wave-underline), (ii) destabilized #15 p40 polypeptide having Cys-177 substituted with Ser-177 (double-underline)(C177S), (iii) destabilized #15 linker “GGGGSKTKQLRVVNGGGGS” containing a urokinase plasminogen activator (uPA) target sequence (SEQ ID NO: 69) (dotted underline), and (iv) destabilized #15 p35 polypeptide having Cys-74 substituted with Ser-74 (double-underline)(C74S).

FIG. 16R: Destabilized IL-12 #16 (DSIL-12 #16) having AA substituted p40 and p35 polypeptides linked as a single chain fusion protein by a long linker containing a neutrophil elastase target sequence (SEQ ID NO:156). The figure shows (i) destabilized #16 p40 predicted 22 AA signal peptide sequence (wave-underline), (ii) destabilized #16 p40 polypeptide having Cys-177 substituted with Ser-177 (double-underline)(C177S), (iii) destabilized #16 linker “GGGGSQEPVKGPVGGGGS” containing a neutrophil elastase target sequence (SEQ ID NO: 70) (dotted underline), and (iv) destabilized #16 p35 polypeptide having Cys-74 substituted with Ser-74 (double-underline)(C74S)

FIG. 16S: Destabilized IL-12 #17 (DSIL-12 #17) having AA substituted p40 and p35 polypeptides linked as a single chain fusion protein by a long linker containing a plasma kallikrein target sequence (SEQ ID NO:157). The figure shows (i) destabilized #17 p40 predicted 22 AA signal peptide sequence (wave-underline), (ii) destabilized #17 p40 polypeptide having Cys-177 substituted with Ser-177 (double-underline)(C177S), (iii) destabilized #17 linker “GGGGSVEKRRNSGPGGGGS” containing a plasma kallikrein target sequence (SEQ ID NO: 71) (dotted underline), and (iv) destabilized #17 p35 polypeptide having Cys-74 substituted with Ser-74 (double-underline)(C74S).

FIG. 16T: Destabilized IL-12 #18 (DSIL-12 #18) having AA substituted p40 and p35 polypeptides linked as a single chain fusion protein by a linker containing plasmin, urokinase plasminogen activator (uPA) and plasma kallikrein target sequences (SEQ ID NO:158). The figure shows (i) destabilized #18 p40 predicted 22 AA signal peptide sequence (wave-underline), (ii) destabilized #18 p40 polypeptide having Cys-177 substituted with Ser-177 (double-underline)(C177S), (iii) destabilized #18 linker “GGSPQFRIKGGKTKQLRVVNVEKRRNSGPGGS” containing plasmin, urokinase plasminogen activator (uPA) and plasma kallikrein target sequences (SEQ ID NO: 72) (dotted underline), (iv) destabilized #18 p35 polypeptide having Cys-74 substituted with Ser-74 (double-underline) (C74S).

FIG. 16U: Destabilized IL-12 #19 (DSIL-12 #19) having AA substituted p40 and p35 polypeptides linked as a single chain fusion protein by a long linker containing several plasmin target sequences (SEQ ID NO:159). The figure shows (i) destabilized #19 p40 predicted 22 AA signal peptide sequence (wave-underline), (ii) destabilized #19 p40 polypeptide having Cys-177 substituted with Ser-177 (double-underline)(C177S), (iii) destabilized #19 linker “GGGSRRPYLKVFNPRRKLEFGGGS” containing several plasmin, target sequences (SEQ ID NO: 73) (dotted underline), and (iv) destabilized #19 p35 polypeptide having Cys-74 substituted with Ser-74 (double-underline)(C74S)

FIG. 16V: Destabilized IL-12 #20 (DSIL-12 #20) having AA substituted p40 and p35 polypeptides linked as a single chain fusion protein by a long linker containing a plasmin target sequence (SEQ ID NO:160). The figure shows (i) destabilized #20 p40 predicted 22 AA signal peptide sequence (wave-underline), (ii) destabilized #20 p40 polypeptide having Cys-177 substituted with Ser-177 (double-underline)(C177S), (iii) destabilized #20 linker “GGGSNSGRAVTYSRSRYLGGGS” containing a plasmin, target sequence (SEQ ID NO: 74) (dotted underline), and (iv) destabilized #20 p35 polypeptide having Cys-74 substituted with Ser-74 (double-underline)(C74S).

FIG. 16W: Destabilized IL-12 #21 (DSIL-12 #21) having AA substituted p40 and p35 polypeptides linked as a single chain fusion protein by a linker containing a tissue plasminogen activator target sequence and a plasmin target sequence (SEQ ID NO:161). The figure shows (i) destabilized #21 p40 predicted 22 AA signal peptide sequence (wave-underline), (ii) destabilized #21 p40 polypeptide having Cys-177 substituted with Ser-177 (double-underline)(C177S), (iii) destabilized #21 linker “GGGSCPGRVVGGPQFRIKGGGGGS” containing a tissue plasminogen activator (tPA) target sequence and a plasmin target sequence (SEQ ID NO: 75) (dotted underline), and (iv) destabilized #21 p35 polypeptide having Cys-74 substituted with Ser-74 (double-underline)(C74S).

FIG. 16X: Destabilized IL-12 #22 (DSIL-12 #22) having AA substituted p40 and p35 polypeptides linked as a single chain fusion protein by a long linker containing a thrombin target sequence (SEQ ID NO:162). The figure shows (i) destabilized #22 p40 predicted 22 AA signal peptide sequence (wave-underline), (ii) destabilized #22 p40 polypeptide having Cys-177 substituted with Ser-177 (double-underline)(C177S), (iii) destabilized #22 linker “GGGSGGGGSLVPRGSSGGGGSGGGGS” containing a thrombin target sequence (SEQ ID NO: 76) (dotted underline), and (iv) destabilized #22 p35 polypeptide having Cys-74 substituted with Ser-74 (double-underline)(C74S).

FIG. 16Y: Destabilized IL-12 #23 (DSIL-12 #23) having AA substituted p40 and p35 polypeptides linked as a single chain fusion protein by a long linker containing a plasmin target sequence (SEQ ID NO:163). The figure shows (i) destabilized #23 p40 predicted 22 AA signal peptide sequence (wave-underline), (ii) destabilized #23 p40 polypeptide having Cys-177 substituted with Ser-177 (double-underline)(C177S), (iii) destabilized #23 linker “GGGSSKGRSLIGGGGS” containing a plasmin target sequence (SEQ ID NO: 77) (dotted underline), and (iv) destabilized #23 p35 polypeptide having Cys-74 substituted with Ser-74 (double-underline)(C74S).

FIG. 16Z: Destabilized IL-12 #24 (DSIL-12 #24) having AA substituted p40 and p35 polypeptides linked as a single chain fusion protein by a long linker containing a plasmin target sequence (SEQ ID NO:164). The figure shows (i) destabilized #24 p40 predicted 22 AA signal peptide sequence (wave-underline), (ii) destabilized #24 p40 polypeptide having Cys-177 substituted with Ser-177 (double-underline)(C177S), (iii) destabilized #24 linker “GGGSQRYKVDYEGGGS” containing a plasmin target sequence (SEQ ID NO: 78) (dotted underline), and (iv) destabilized #24 p35 polypeptide having Cys-74 substituted with Ser-74 (double-underline)(C74S).

FIG. 16AA: Destabilized IL-12 #25 (DSIL-12 #25) having AA substituted p40 and p35 polypeptides linked as a single chain fusion protein by a long linker containing a plasmin target sequence (SEQ ID NO:165). The figure shows (i) destabilized #25 p40 predicted 22 AA signal peptide sequence (wave-underline), (ii) destabilized #25 p40 polypeptide having Cys-177 substituted with Ser-177 (double-underline)(C177S), (iii) destabilized #25 linker “GGGSPQSRSVPPGGGS” containing a plasmin target sequence (SEQ ID NO: 79) (dotted underline), and (iv) destabilized #25 p35 polypeptide having Cys-74 substituted with Ser-74 (double-underline)(C74S).

FIG. 16BB: Destabilized IL-12 #26 (DSIL-12 #26) having AA substituted p40 and p35 polypeptides linked as a single chain fusion protein by a long linker containing a plasmin target sequence (SEQ ID NO:166). The figure shows (i) destabilized #26 p40 predicted 22 AA signal peptide sequence (wave-underline), (ii) destabilized #26 p40 polypeptide having Cys-177 substituted with Ser-177 (double-underline)(C177S), (iii) destabilized #26 linker “GGGSKKPRCGVPGGGS” containing a plasmin target sequence (SEQ ID NO: 80) (dotted underline), and (iv) destabilized #26 p35 polypeptide having Cys-74 substituted with Ser-74 (double-underline)(C74S).

FIG. 16CC: Destabilized IL-12 #27 (DSIL-12 #27) having AA substituted p40 and p35 polypeptides linked as a single chain fusion protein by a plasmin target site linker (SEQ ID NO:167). The figure shows (i) destabilized #27 p40 predicted 22 AA signal peptide sequence (wave-underline), (ii) destabilized #27 p40 polypeptide having Cys-177 substituted with Ser-177 (double-underline)(C177S) positions 258-264 modified to “ASGREPI” (SEQ ID NO: 81), (dashed underline), (iii) destabilized #27 linker “PQFRIKGG” containing a plasmin target sequence (SEQ ID NO: 62) (dotted underline), (iv) destabilized #27 p35 polypeptide having Cys-74 substituted with Ser-74 (double-underline)(C74S).

FIG. 16DD: Single-chain (non-destabilized) IL-12 #28 (SCIL-12 #28) having p40 and p35 polypeptides linked as single chain fusion protein by a thrombin target sequence (SEQ ID NO:168). The figure shows (i) single-chain IL-12 #28 p40 predicted 22 AA signal peptide sequence (wave-underline), (ii) single-chain IL-12 #28 p40 polypeptide having wild-type Cys-177 (double-underline), (iii) single-chain IL-12 #28 linker “LVPRGSS” containing a thrombin target sequence (SEQ ID NO: 60) (dotted underline), and (iv) single-chain IL-12 #28 p35 polypeptide having wild-type Cys-74 (double-underline) and wild-type Arg-189 (double-underline).

FIG. 16EE: Single-chain (non-destabilized) IL-12 #29 (SCIL-12 #29) having p40 and p35 polypeptides linked as single chain fusion protein by a long linker containing a thrombin target sequence (SEQ ID NO:169). The figure shows (i)single-chain IL-12 #29 p40 predicted 22 AA signal peptide sequence (wave-underline), (ii) single-chain IL-12 #29 p40 polypeptide having wild-type Cys-177 (double-underline), (iii) single-chain IL-12 #29 linker “GGGGSLVPRGSSGGGGS” containing a thrombin target sequence (SEQ ID NO: 68) (dotted underline), and (iv) single-chain IL-12 #29 p35 polypeptide having wild-type Cys-74 (double-underline) and wild-type Arg-189 (double-underline)

FIG. 16FF: Single-chain (non-destabilized) IL-12 #30 (SCIL-12 #30) having p40 and p35 polypeptides linked as single chain fusion protein by an extended long linker containing a thrombin target sequence (SEQ ID NO:170). The figure shows (i) single-chain IL-12 #30 p40 predicted 22 AA signal peptide sequence (wave-underline), (ii) single-chain IL-12 #30 p40 polypeptide having wild-type Cys-177 (double-underline), (iii) single-chain IL-12 #30 linker “GGGSGGGGSLVPRGSSGGGGSGGGGS” containing a thrombin target sequence (SEQ ID NO: 76) (dotted underline), and (iv) single-chain IL-12 #30 p35 polypeptide having wild-type Cys-74 (double-underline) and wild-type Arg-189 (double-underline).

FIG. 17 presents immunoblot (a.k.a., Western Blot) analysis to detect thrombin cleavage (IL-12 p35 antibody). HEK293T cells were transfected with the indicated constructs in serum-free media (SFM). 72 hours post-transfection, cell supernatants were collected and treated with 67 U/mL thrombin for 20 hours at 37 degrees C. Cleavage of IL-12 by thrombin was assessed by immunoblotting (a.k.a., western blotting) under reducing (+DTT) and non-reducing (−DTT) conditions using an antibody against the p35 subunit of IL-12. Under reducing conditions, thrombin cleavage of all three IL-12 mutants was detected. In contrast, under non-reducing conditions, thrombin cleavage of Destabilized #4 was not detected. These data suggest that although Destabilized #4 is cut by thrombin, the disulfide bonds present in the IL-12 molecule keep the protein in its native state. However, when the disulfide bonds of IL-12 are disrupted, either by exposure to DTT or by mutagenesis (Destabilized #2 and Destabilized #5), the activity of thrombin is sufficient cleave and inactivate the single chain IL-12.

FIG. 18 presents immunoblot (a.k.a., Western Blot) analysis to detect thrombin cleavage (IL-12 p40 antibody). HEK293T cells were transfected with the indicated constructs in serum-free media (SFM). 72 hours post-transfection, cell supernatants were collected and treated with 67 U/mL thrombin for 20 hours at 37 degrees C. Cleavage of IL-12 by thrombin was assessed by immunoblotting (a.k.a., western blotting) under reducing (+DTT) and non-reducing (−DTT) conditions using an antibody against the p40 subunit of IL-12. Under reducing conditions, thrombin cleavage of all three IL-12 mutants was detected. In contrast, under non-reducing conditions, thrombin cleavage of Destabilized #4 was not detected. These data are consistent with the results of the p35 western blot and again indicate the importance of disulfide bond disruption in thrombin-mediated IL-12 cleavage.

FIG. 19 demonstrates decreased interferon-gamma (IFNg) production from destabilized IL-12 in the presence of Thrombin. To determine if the cleavage of IL-12 observed in the above western blots was sufficient to inhibit IL-12 activity, serial dilutions of the thrombin-digested supernatants from the transfected HEK293T cells were added to NK-92 cells, and the secretion of interferon gamma (IFNg) was measured by HTRF. In the absence of thrombin treatment, all 3 destabilized IL-12 mutants showed activity similar to that of wildtype (WT) IL-12. However, when treated with thrombin, Destabilized #2 and Destabilized #5 showed a significant reduction in activity compared to WT, whereas Destabilized #4 did not. The same results were observed when the thrombin digestions were carried out in the absence (data not shown) and presence of serum (10% FBS). The data indicate that both thrombin cleavage and destabilization of the disulfide bond bridging the p35 and p40 subunits were required to reduce the activity of IL-12.

FIG. 20 presents thrombin dose titration for reducing IFNg production in NK-92 cells. To determine the minimal amount of thrombin required to inactivate Destabilized #2 and #5, supernatants from the transfected HEK293T cells were digested with varying amounts of thrombin and then added to NK-92 cells. As a measure of IL-12 activity, the secretion of IFNg from the NK-92 cells was determined by HTRF (Homogeneous Time Resolved Fluorescence). In the absence of serum, approximately 10 U/mL thrombin was needed to significantly reduce the activity of both mutants. Approximately 10U/mL thrombin was required for biological inactivation of Destabilized #2 and Destabilized #5.

FIG. 21 presents expression data of destabilized IL-12 in primary human T cells. To demonstrate that destabilized IL-12 variants can be expressed by primary human T cells, transient transfection of T cells were performed. Briefly, human PBMCs from a normal healthy donor were nucleofected using the Amaxa nucleofection device. A total of 15 ug of each respective DNA was used to nucleofect 20e6 PBMCs. GFP expressing vector was utilized to assess transfection efficiency as assessed by flow cytometry on gated CD3 T cell populations. Cells were cultured for ˜48 hrs. Culture supernatants were collected and an IL-12p70 ELISA performed to detect protein expression levels. To also confirm the transfected T cells were expressing the IL-12, intracellular cytokine staining (ICS) was performed. For the last 6 hrs of culture, Brefeldin A was added to the cultures to prevent protein transport then cells were collected and stained for cell surface markers. Cells were also prestained with a cell viability marker. Cells were then fixed using a 4% PFA solution then permeabilized and stained with anti-human IL-12p40 and anti-human IL-12p35 subunit antibodies. Samples were analyzed on a LSR II flow cytometer with gating performed on FSC/SSC/LIVE/CD3 gated cell population. Overall the data from the ELISA and ICS demonstrated dslL-12 versions #2 and #5 typically had lower levels of expression in comparison to dslL-12 version 4; but nevertheless, IL-12 expression was confirmed in the transfected cells. Destabilized #2 and #5 constructs expression levels by ELISA may be under-represented due to limited antibody binding affinity.

FIG. 22 and FIG. 23 present data of NK-92 activity assays of destabilization mutants (Trials #1 and #2) following thrombin digestion After digestion with thrombin, the data indicate that destabilized IL-12 #2 (DSIL-12 #2) exhibits approximately 11-fold reduction in biological activity, Destabilized IL-12 #5 (DSIL-12 #5) exhibits approximately 9-fold reduction in biological activity, and Destabilized IL-12 #4 (DSIL-12 #4) exhibits approximately 1.5 fold reduction in biological activity compared to the corresponding wild-type single-chain IL-12 molecule.

FIG. 24 presents the results of immunoblot (a.k.a., Western blot) analysis which demonstrates that linker length contributes to thrombin sensitivity of destabilized single-chain IL-12 proteins.

FIG. 25 presents the results of immunoblot (a.k.a., Western blot) analysis which demonstrates that linker length contributes to plasmin sensitivity of destabilized single-chain IL-12 proteins.

FIG. 26 presents the results of immunoblot (a.k.a., Western blot) analysis of the digestion of various destabilized single-chain IL-12 proteins in human plasma.

FIG. 27 presents the results of immunoblot (a.k.a., Western blot) analysis of the digestion of destabilized single-chain IL-12 proteins in human plasma over time.

FIG. 28A represents data from a bioactivity assay of various destabilized single-chain IL-12 proteins. FIG. 28B shows the concentration for half maximal response (EC50) of the proteins used in the assays.

FIG. 29A represents pharmacokinetic analysis of destabilized single-chain IL-12 proteins. FIG. 29B represents pharmacokinetic analysis of destabilized single-chain IL-12 proteins by measuring serum activity on NK-92 cells.

FIG. 30 represents pharmacokinetic analysis of destabilized single-chain IL-12 proteins containing or lacking proteolytic target sequences in their linkers.

### DETAILED DESCRIPTION OF THE INVENTION

The present invention advantageously provides destabilized modified forms of IL-12, including modified forms of naturally-occurring, heterodimeric p35/p40 forms of IL-12 and of traditional single-chain forms of IL-12 (such as, traditional p40-linker-p35 and p35-linker-p40 forms of IL-12; as well as modified “topo” single chain (topologically manipulated) forms of IL-12 as described herein). These destabilized modified forms of IL-12 are engineered (e.g., by genetic, recombinant and synthetic engineering technologies) to have a shortened in vivo half-life compared to that of a corresponding non-modified form of IL-12.

The present invention advantageously provides destabilized modified forms of membrane bound IL-12 (“mbIL-12”), including modified forms of membrane bound heterodimeric p35/p40 forms of IL-12 and membrane bound single-chain forms of IL-12 (such as, traditional p40-linker-p35 and p35-linker-p40 forms of IL-12; as well as membrane bound forms of single chain (topologically manipulated) forms of IL-12 as described herein) wherein the modified forms further comprise a membrane-anchoring moiety or amino acid sequence (i.e., as a fusion protein) wherein membrane-anchoring portion functions to localize (or “co-localize”) the biologically active IL-12 molecule to the extracellular side of a cell membrane. Membrane anchoring moieties may comprise any amino acid sequence or organic molecule useful in anchoring, tethering or linking IL-12 polypeptide(s) to a mammalian cell membrane.

Destabilizedmodified IL-12 molecules of the invention include membrane binding (i.e., anchoring, linking, or tethering) moieties selected from the group consisting of: covalent membrane surface linking moieties, hydrophobic membrane surface linking moieties, hydrophilic membrane surface linking moieties, ionic membrane surface linking moieties, integral cell membrane polypeptides, and transmembrane polypeptides. Useful anchoring, tethering, or linking moieties for generating membrane bound forms of IL-12 of the invention include both naturally occurring and artificially created/synthesized transmembrane or cell membrane-embedding amino acid sequences capable of sequestering (i.e., anchoring, tethering, linking) biologically active IL-12 molecules to the extracellular side (surface) of a cell membrane.

Short half-life (destabilized modified) forms of IL-12 provide greater therapeutic control for in vivo therapeutic delivery, in particular when used in combination with ligand inducible delivery of IL-12.

Destabilized modified forms of IL-12 as described herein are engineered to have plasma proteinase cleavage sites. Multiple locations exist on IL-12 to engineer proteinase cleavage sites. Cleavage sites are engineered into the IL-12 p35 domain, the IL-12 p40 domain, or both the IL-12 p35 and p40 domain, in any of heterodimeric or single-chain forms of IL-12. For single chain forms of IL-12, in addition to, or instead of, the p35 and p40 subunits, proteinase cleavage sites are engineered into linker sequences used to generate single chain IL-12 fusion proteins, or engineered into amino acid sequences used to generate membrane tethered IL-12 (mbIL-12). Modified forms of IL-12 are engineered to be rapidly cleared from the in vivo blood plasma.

### Examples of Proteinases

A proteinase (also referred to herein and elsewhere in the art as a protease or peptidase) is an enzyme that cleaves amino acid bonds (an action referred to as “proteolysis”) in a protein or polypeptide (as these terms may be used synonymously herein). Typically, proteinases perform proteolysis by hydrolysis of the peptide bonds that link amino acids together in the polypeptide chain. For example, there are currently at least six identified classes of proteinases. These are: (1) Serine proteases (utilize a serine alcohol for proteolysis); (2) Threonine proteases (utilize a threonine secondary alcohol); (3) Cysteine proteases (utilize a cysteine thiol); (4) Aspartate proteases (utilize an aspartate carboxylic acid); (5) Glutamic acid proteases (utilize a glutamate carboxylic acid); and, (6) Metalloproteases (utilize a metal ion, usually zinc). For further information on proteinases, see for example, “Molecular Biology of the Cell” 5th Edition (2007) by Alberts et al. (ISBN #9780815341055; Garland Publishing Inc., New York & London); see also, “Biochemistry” 4th Edition (2010) by Voet & Voet (ISBN #978-0470570951; Wiley & Sons, NY).

Some examples of well-known proteases (for purposes of exemplification and illustration only and not by way of limitation) include matrix metalloproteinase-2 (MMP-2), plasmin, thrombin, urokinase-type plasminogen activator (uPA), and carboxy peptidases (e.g., acting in concert with enteropeptidases or endoproteinases).

### WP-2

Matrix metalloproteinase-2 (MMP-2) is a 72 kDa protein (also known as Type IV Collagenase and Gelatinase A). Proteins in this family function to breakdown extracellular matrix components (e.g., type IV collagen—a main structural component of basement membranes) in normal physiological processes; such as embryonic development, reproduction, and tissue remodeling, as well as in disease processes, such as arthritis and metastasis. Most MMP's are secreted as inactive proproteins which are activated when cleaved by extracellular proteinases. See e.g., Devarajan, et al. “Structure and expression of neutrophil gelatinase cDNA. Identity with type IV collagenase from HT1080 cells”. J. Biol. Chem. 267 (35): 25228-32 (December 1992); Massova, et al. “Matrix metalloproteinases: structures, evolution, and diversification”. FASEB J. 12 (12): 1075-1095 (1998); Nagase et al. “Matrix metalloproteinases”. J. Biol. Chem. 274 (31): 21491-21494 (1999); and, Hrabec et al. “[Type IV collagenases (MMP-2 and MMP-9) and their substrates—intracellular proteins, hormones, cytokines, chemokines and their receptors]”. Postepy Biochem. 53 (1): 37-45 (2007).

### Plasmin

Plasmin is a serine protease which plays a critical role in dissolving fibrin blood clots (referred to as fibrinolysis), it proteolyzes other proteases to convert them to active form, such as collagenases and some mediators of the complement system. Plasmin is known to cleave fibrin, fibronectin, thrombospondin, laminin, and von Willebrand factor. Plasmin is released as a zymogen called plasminogen (PLG) from the liver into the systemic circulation. In the blood plasma circulation, plasminogen is found in a closed, activation resistant conformation. Upon binding to clots, or to a cell surface, plasminogen changes to an open form which can be converted into active plasmin by a variety of enzymes, such as tissue plasminogen activator (tPA), urokinase plasminogen activator (uPA), kallikrein, and factor XII (Hageman factor). See e.g., Butera, et al. “Characterization of a reduced form of plasma plasminogen as the precursor for angiostatin formation” J. Biol. Chem., 289(5):2992-3000 (Jan. 31, 2014); Forsgren et al. “Molecular cloning and characterization of a full-length cDNA clone for human plasminogen” FEBS Lett. 213 (2): 254-60 (1987); and, Law et al. “The X-ray Crystal Structure of Full-Length Human Plasminogen” Cell Reports 1 (3): 185 (2012).

### Thrombin

Thrombin is a serine protease which is sometimes also called fibrinogenase, thrombase, thrombofort, topical, thrombin-C, tropostasin, activated blood-coagulation factor II, blood-coagulation factor IIa, factor IIa, E thrombin, beta-thrombin, gamma-thrombin. Prothrombin (or coagulation factor II) is proteolytically cleaved to form thrombin in the coagulation cascade, which ultimately results in the reduction of blood loss. Thrombin in turn acts as a serine protease that converts soluble fibrinogen into insoluble strands of fibrin, as well as catalyzing many other coagulation-related reactions. See e.g., Royle et al. “Human genes encoding prothrombin and ceruloplasmin map to 11p11-q12 and 3q21-24, respectively”. Somat. Cell Mol. Genet. 13 (3): 285-92 (May 1987); Degen et al. “Nucleotide sequence of the gene for human prothrombin”. Biochemistry 26 (19): 6165-77 (September-1987); De Cristofaro et al. “Thrombin domains: structure, function and interaction with platelet receptors”. J. Thromb. Thrombolysis 15 (3): 151-63 (2004); Bode et al. “Structure and interaction modes of thrombin”. Blood Cells Mol. Dis 36 (2): 122-30 (2007); and, Wolberg et al. “Thrombin generation and fibrin clot structure”. Blood Rev 21 (3): 131-42 (2007).

Urokinase-Type Plasminogen Activator (uPA)

Urokinase-type plasminogen activator (uPA), is a serine protease first isolated from human urine in 1947. uPA is also relatively abundant, however, in the blood stream and extracellular matrix. The primary physiological substrate is plasminogen, which is an inactive form (zymogen) of the serine protease plasmin. Activation of plasmin triggers a proteolysis cascade that, depending on the physiological environment, participates in thrombolysis or extracellular matrix degradation. See e.g., Crippa “Urokinase-type plasminogen activator” Intl. J. Biochem. & Cell Biol. 39:4, 600-694 (2007).

### Carboxypeptidases

Carboxypeptidases are proteases that hydrolyze peptide bonds at the carboxy-terminal (C-terminal) end of a protein or peptide. Carboxypeptidases function in blood clotting, growth factor production, wound healing, reproduction, and many other processes. Carboxypeptidases are usually classified into one of the six known families of proteases based on their active site mechanism. For example, carboxypeptidases that use a metal ion in the active site are called “metallo-carboxypeptidases”; carboxypeptidases that utilize serine residues at the active site are called “serine carboxypeptidases”; and, those that utilize cysteine at the active site are called “cysteine carboxypeptidases” (or “thiol carboxypeptidases”). Another classification system for carboxypeptidases is based on their substrate preference. For example in this system, carboxypeptidases that preferentially target amino acids having aromatic or branched hydrocarbon chains are called carboxypeptidase A (“A” being for aromatic/aliphatic). Carboxypeptidases that cleave positively charged amino acids (arginine, lysine) are called carboxypeptidase B (“B” for basic). Some, but not all, carboxypeptidases are initially produced in an inactive form, referred to as a procarboxypeptidase; these may be converted to an active form via cleavage by enteropeptidases or endopeptidases. For example, the inactive zymogen form of pancreatic carboxypeptidase A (called “pro-carboxypeptidase A”) is converted to its active form by an enteropeptidase (thereby ensuring that cells in which pro-carboxypeptidase A is produced are not themselves digested). See e.g., Section on “Proteases” in Berg et al. “Biochemistry” 5^{th} Edition, W.H. Freeman, NY (2002).

### Single Chain IL-12

The present invention advantageously provides (as a foundation for generating shortened half-life IL-12 compositions) isolated polynucleotides encoding destabilized topologically manipulated single chain IL-12 (scIL-12) polypeptides, such as p40N-p35-p40C scIL-12 as described in international patent application PCT/US2014/70695 (WO2015/095249) “Single Chain IL-12 Nucleic Acids, Polypeptides, And Uses Thereof” which is hereby incorporated by reference in its entirety. In one embodiment, such destabilized “topo” scIL-12 polypeptides are modified to comprise one or more amino acid substitution and one or more proteolytic amino acid sequences, thereby rendering the biologically active composition susceptible to reduced in vivo (e.g., in blood plasma) half-life and/or reduced biological activity. The polynucleotides and polypeptides of the present invention are useful in methods of enhancing the immune response of a host, for example as vaccine adjuvants, and in the treatment of proliferative disorders such as cancer, infectious diseases, and immune system disorders.

### Membrane Bound IL-12

IL-12 systemic toxicity is also limited or more tightly controlled via mechanisms involving tethering IL-12 to the cell surface so it acts locally, at the site of the tumor, but is inhibited or prevented from circulating systemically. Literature reports have shown IL-12 can be anchored to the cell surface through attachment of a glycosyl-phosphatidylinositol (GPI) signal peptide to the C-terminus of scIL-12 (Nagarajan 2002, Bozeman 2013) as well as with the CD80 transmembrane domain (TMD) (Pan 2012).

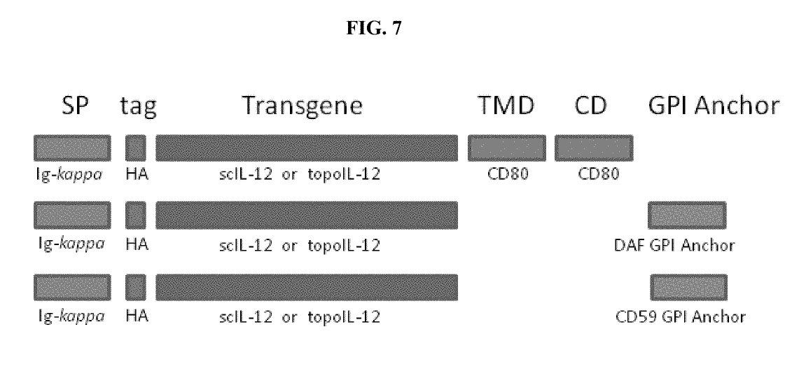

Embodiments of the present invention include both destabilized GPI anchored and TMD membrane-tethered (anchored/membrane-bound) forms of scIL-12 and topoIL-12. See, for example, but without limitation, constructs depicted by FIG. 7.

In certain embodiments, the invention provides membrane bound forms of IL-12 (mbIL-12) which confer highly localized therapeutic effects.

In certain embodiments, mbIL-12 is a single chain IL-12 molecule such as in any of the forms described or referenced herein.

In certain embodiments, mbIL-12 are engineered to comprise protease sensitive sites (proteolytic sites) as described herein.

In certain embodiments, mbIL-12 comprising engineered protease sensitive sites is a single chain IL-12 molecule such as in any of the forms described or referenced herein.

Any number of transmembrane domains (TMD) selected from a multitude of naturally occurring TMD may be incorporated to generate mbIL-12 polypeptides of the invention. There are two basic types of transmembrane proteins: alpha-helical and beta-barrels. Alpha-helical proteins are present in the plasma membrane of eukaryotes and, in humans, as much as 27% of all proteins may be alpha-helical membrane proteins. Indeed, one survey of the entire human membrane proteome determined there are at least 2,925 unique integral alpha-helical TMD sequences encoded by the human genome (Pieper, et al., “Coordinating the impact of structural genomics on the human α-helical transmembrane proteome”, Nat Struct Mol Biol. 20(2): 135-138 (2013).

All beta-barrel transmembrane proteins have simplest up-and-down topology, which may reflect their common evolutionary origin and similar folding mechanism. TMP classification by topology refers to the position of the N- and C-terminal domains. Types I, II, and III are single-pass molecules, while type IV are multiple-pass molecules. Type I transmembrane proteins are anchored to the lipid membrane with a stop-transfer anchor sequence and have their N-terminal domains targeted to the ER lumen during synthesis (and the extracellular space, if mature forms are located on cell surface). Type II and III are anchored with a signal-anchor sequence, with type II being targeted to the ER lumen with its C-terminal domain, while type III have their N-terminal domains targeted to the ER lumen. Type IV TMP are subdivided into IV-A, with their N-terminal domains targeted to the cytosol and IV-B, with an N-terminal domain targeted to the lumen.

Some non-limiting examples of the types of known TMD which could be utilized include those from: Single-pass transmembrane proteins (TMP) (including: Type-I TMP such as E3 Ubiquitin-Protein Ligase; Type-II TMP such as 4F2 Cell-Surface Antigen Heavy Chain; Type-III TMP such as Linker For Activation of T-cells Family Member 1; and, Type-IV TMP such as Junctophilin-1); Multi-pass TMP such as human Calcitonin Receptor; and, Beta-barrel TMP. See e.g., Almén, et al., “Mapping the human membrane proteome: A majority of the human membrane proteins can be classified according to function and evolutionary origin”. BMC Biol. 7:50 (2009).

### Definitions

The following defined terms are used throughout the present specification, and should be helpful in understanding the scope and practice of the present invention.

The term “destabilized IL-12” and abbreviation “DSIL-12”, or variations of these, as used herein means any form of IL-12 (including, but not limited to, forms such as those referenced immediately below) wherein portions of naturally occurring IL-12 p40 and/or IL-12 p35 polypeptide sequences comprise at least one or more amino acid substitutions, deletions, or insertions to effect a change in stability of IL-12 polypeptide folding, dimerization or affinity interactions (i.e., of p35 and p40 subunits; whether p35 and p40 are separate polypeptides or linked as a single-chain fusion protein), or biological activity wherein the polypeptide(s) also comprise at least one or more proteolytic amino acid sequences (i.e., amino acid substitutions, deletions, or insertions which enhance or increase susceptibility to proteolytic degradation).

The term “traditional single chain IL-12” or “traditional scIL-12” as used herein means forms of single chain IL-12 which have been engineered to express the IL-12 p40 polypeptide fused via a linker sequence to the IL-12 p35 polypeptide such that the p40/p35 molecule is produced as a single polypeptide chain. This “traditional scIL-12” configuration can be in either order such that the single polypeptide is produced beginning with the p40 polypeptide as the amino-terminal portion (“N-terminal”) linked (via linker polypeptide) to the p35 polypeptide as the carboxyl-terminal portion (“C-terminal”). This traditional configuration may be represented by a shorthand designation as “p40-linker-p35”. Conversely, in a traditional scIL-12 construct, the p35 portion can also be the N-terminal portion linked to p40 as the C-terminal portion in a format designated as “p35-linker-p40”.

The term “topologically manipulated scIL-12” or “topo scIL-12” or “topo IL-12” as used herein means a form of single chain IL-12 where the p40 IL-12 polypeptide has been engineered to comprise within its linear sequence (or be “interrupted” by) the p35 IL-12 polypeptide, as described more fully elsewhere herein. This “topo IL-12” configuration may be represented herein by short hand as “p40N-p35-p40C”, thereby indicating that an N-terminal portion of the p40 polypeptide has linked to it (via a short linker or no linker) the p35 polypeptide, which is then fused to the remainder of a carboxy-terminal portion of p40 (via a short linker or no linker).

Unless indicated or specified otherwise, the term “scIL-12” or “single chain IL-12” as used herein means both “traditional” and “topologically manipulated” scIL-12.

Unless indicated or specified otherwise, the terms “IL-12” or “IL-12 compositions of the invention” (and apparent variations of these terms) are intended to mean and encompass heterodimeric IL-12 polypeptide complexes as well as both “traditional” and “topologically manipulated” scIL-12.

Unless indicated or specified otherwise, “membrane bound IL-12” or “mbIL-12” means IL-12 polypeptides comprising a membrane anchoring moiety and/or amino acid sequence (i.e., IL-12 fusion proteins) which function to localize (or “co-localize”, “tether”, or “anchor”) the IL-12 molecule to the extracellular side of a cell membrane.

Unless indicated or specified otherwise, “ . . . of the invention” (or similar phrases) when used in association with, or reference to, IL-12 polypeptides, polynucleotides, and amino acid sequences described herein means molecules which have been engineered (e.g., synthetically, genetically, recombinantly) to comprise altered amino acid residues compared to an initial or corresponding wild-type or naturally occurring IL-12 polypeptide sequence such that the modification results in reduced half-life of IL-12 biological activity.

Unless indicated or specified otherwise, the terms “modified” in relation to “IL-12”, “scIL-12” and “mbIL-12” means IL-12 polypeptides which have been engineered (e.g., synthetically, genetically, recombinantly) to comprise altered amino acid residues compared to an initial or corresponding wild-type or naturally occurring IL-12 polypeptide sequence such that the modification results in reduced half-life of IL-12 biological activity. In a specific embodiment, when necessary and possible to attach a numeric value, the term “about” or “approximately” means within 10% of a given value or range.

The term “substantially free” means that a composition comprising “A” (where “A” is a single protein, DNA molecule, vector, recombinant host cell, etc.) is substantially free of “B” (where “B” comprises one or more contaminating proteins, DNA molecules, vectors, etc.) when at least about 75% by weight of the proteins, DNA, vectors (depending on the category of species to which A and B belong) in the composition is “A”. Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

The term “isolated” for the purposes of the present invention designates a biological material (nucleic acid or protein) that has been removed, at some point, from its original environment (the environment in which it is naturally present). For example, a polynucleotide present in the natural state in a plant or an animal is not isolated, however the same polynucleotide separated from the adjacent nucleic acids in which it is naturally present, is considered “isolated”. The term “purified” does not require the material to be present in a form exhibiting absolute purity, exclusive of the presence of other compounds. It is rather a relative definition.

A polynucleotide is in the “purified” state after purification of the starting material or of the natural material by at least one order of magnitude, preferably 2 or 3 and preferably 4 or 5 orders of magnitude.

As used herein, the term “substantially pure” describes a polypeptide or other material which has been separated from its native contaminants. Typically, a monomeric polypeptide is substantially pure when at least about 60 to 75% of a sample exhibits a single polypeptide backbone. Minor variants or chemical modifications typically share the same polypeptide sequence. Usually a substantially pure polypeptide will comprise over about 85 to 90% of a polypeptide sample, and preferably will be over about 99% pure. Normally, purity is measured on a polyacrylamide gel, with homogeneity determined by staining. Alternatively, for certain purposes high resolution will be necessary and HPLC or a similar means for purification will be used. For most purposes, a simple chromatography column or polyacrylamide gel will be used to determine purity.

The term “substantially free of naturally-associated host cell components” describes a polypeptide or other material which is separated from the native contaminants which accompany it in its natural host cell state. Thus, a polypeptide which is chemically synthesized or synthesized in a cellular system different from the host cell from which it naturally originates will be free from its naturally-associated host cell components.

The terms “nucleic acid” or “polynucleotide” are used interchangeably herein to refer to a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes but is not limited to cDNA, genomic DNA, plasmid DNA, synthetic DNA, and semi-synthetic DNA. DNA may be linear, circular, or supercoiled.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes, without limitation, double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “fragment” will be understood to mean a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 6-1500 consecutive nucleotides of a nucleic acid according to the invention.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A “gene” refers to an assembly of nucleotides that encode an RNA transcript or a polypeptide, and includes cDNA and genomic DNA nucleic acids. “Gene” also refers to a nucleic acid fragment that expresses a specific protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Heterologous” DNA refers to DNA not naturally located in the cell, or in a chromosomal site of the cell. Preferably, the heterologous DNA includes a gene foreign to the cell.

The term “genome” includes chromosomal as well as mitochondrial, chloroplast and viral DNA or RNA.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989 infra). Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization.

Stringency conditions can be adjusted to screen for moderately similar fragments, such as homologous sequences from distantly related organisms, to highly similar fragments, such as genes that duplicate functional enzymes from closely related organisms. For preliminary screening for homologous nucleic acids, low stringency hybridization conditions, corresponding to a Tₘ of 55°, can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC.

As used herein, the term “oligonucleotide” refers to a nucleic acid, generally of at least 18 nucleotides, that is hybridizable to a genomic DNA molecule, a cDNA molecule, a plasmid DNA or an mRNA molecule. Oligonucleotides can be labeled, e.g., with ³²P-nucleotides or nucleotides to which a label, such as biotin, has been covalently conjugated. A labeled oligonucleotide can be used as a probe to detect the presence of a nucleic acid. Oligonucleotides (one or both of which may be labeled) can be used as PCR primers, either for cloning full length or a fragment of a nucleic acid, or to detect the presence of a nucleic acid. An oligonucleotide can also be used to form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “primer” is an oligonucleotide that hybridizes to a target nucleic acid sequence to create a double stranded nucleic acid region that can serve as an initiation point for DNA synthesis under suitable conditions. Such primers may be used in a polymerase chain reaction.

“Polymerase chain reaction” is abbreviated PCR and means an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid, and extension of the annealed primer(s) by DNA polymerase. PCR provides a means to detect the presence of the target molecule and, under quantitative or semi-quantitative conditions, to determine the relative amount of that target molecule within the starting pool of nucleic acids.

“Reverse transcription-polymerase chain reaction” is abbreviated RT-PCR and means an in vitro method for enzymatically producing a target cDNA molecule or molecules from an RNA molecule or molecules, followed by enzymatic amplification of a specific nucleic acid sequence or sequences within the target cDNA molecule or molecules as described above. RT-PCR also provides a means to detect the presence of the target molecule and, under quantitative or semi-quantitative conditions, to determine the relative amount of that target molecule within the starting pool of nucleic acids.

A DNA “coding sequence” is a double-stranded DNA sequence that is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, without limitation, promoters, translation leader sequences, introns, polyadenylation recognition sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

“Open reading frame” is abbreviated ORF and means a length of nucleic acid sequence, either DNA, cDNA or RNA, that comprises a translation start signal or initiation codon, such as an ATG or AUG, and a termination codon and can be potentially translated into a polypeptide sequence.

Many methods known in the art may be used to propagate a polynucleotide according to the invention. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus, adenovirus and adeno-associated virus (AAV); insect viruses such as baculovirus; yeast vectors; bacteriophage vectors (e.g., lambda); and plasmid and cosmid DNA vectors, to name but a few.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. A large number of vectors known in the art may be used to manipulate nucleic acids, incorporate response elements and promoters into genes, etc. Possible vectors include, for example but without limitation, plasmids or modified viruses including, for example bacteriophages such as lambda derivatives, or plasmids such as pBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include but are not limited to retrovirus, adeno-associated virus (AAV), pox, baculovirus, vaccinia, herpes simplex, Epstein-Barr, adenovirus, geminivirus, and caulimovirus vectors. Non-viral vectors include, without limitation, plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A “cloning vector” is a “replicon”, which is a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (“shuttle vector”).

Vectors may be introduced into the desired host cells by methods known in the art, e.g., transfection, electroporation, microinjection, transduction, cell fusion, DEAE dextran, calcium phosphate precipitation, lipofection (lysosome fusion), particle bombardment, use of a gene gun, or a DNA vector transporter (see, e.g., Wu et al., 1992, J. Biol. Chem. 267:963-967; Wu and Wu, 1988, J. Biol. Chem. 263:14621-14624; and Hartmut et al., Canadian Patent Application No. 2,012,311, filed Mar. 15, 1990).

A polynucleotide according to the invention can also be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et al., 1987. PNAS 84:7413; Mackey, et al., 1988. Proc. Natl. Acad. Sci. U.S.A. 85:8027-8031; and Ulmer et al., 1993. Science 259:1745-1748). The use of cationic lipids may promote encapsulation of negatively charged nucleic acids, and also promote fusion with negatively charged cell membranes (Feigner and Ringold, 1989. Science 337:387-388). Particularly useful lipid compounds and compositions for transfer of nucleic acids are described in International Patent Publications WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce a vector in vivo as a naked DNA plasmid (see U.S. Pat. Nos. 5,693,622, 5,589,466 and 5,580,859). Receptor-mediated DNA delivery approaches can also be used (Curiel et al., 1992. Hum. Gene Ther. 3:147-154; and Wu and Wu, 1987. J. Biol. Chem. 262:4429-4432).

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

“Transformation” refers to the transfer of a nucleic acid molecule into a host cell or into the genome of a host organism, resulting in genetically stable or instable inheritance. Host organisms containing the transformed nucleic acid molecule stably integrated into the host organism genome are referred to as “transgenic” or “recombinant” or “transformed” organisms. Cells containing the transformed nucleic acid molecule are referred to as “transformed.” Cells containing the transformed nucleic acid molecule stably integrated into the host cell genome are referred to as “transformed” or “stably transformed.” Cells containing the transformed nucleic acid molecule which is not stably integrated into the host cell genome are referred to as “transiently transformed” or “transiently transfected”.

The term “genetic region” will refer to a region of a nucleic acid molecule or a nucleotide sequence that comprises a gene encoding a polypeptide.

In addition, the recombinant vector comprising a polynucleotide according to the invention may include one or more origins for replication in the cellular hosts in which their amplification or their expression is sought, markers or selectable markers.

The term “selectable marker” means an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include, without limitation: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Selectable marker genes may also be considered reporter genes.

The term “reporter gene” means a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include, without limitation: luciferase (Luc), green fluorescent protein (GFP), chloramphenicol acetyltransferase (CAT), β-galactosidase (LacZ), β-glucuronidase (Gus), and the like.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. Promoters that cause a gene to be expressed in a specific cell type are commonly referred to as “cell-specific promoters” or “tissue-specific promoters”. Promoters that cause a gene to be expressed at a specific stage of development or cell differentiation are commonly referred to as “developmentally-specific promoters” or “cell differentiation-specific promoters”. Promoters that are induced and cause a gene to be expressed following exposure or treatment of the cell with an agent, biological molecule, chemical, ligand, light, or the like that induces the promoter are commonly referred to as “inducible promoters” or “regulatable promoters”. It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “response element” means one or more cis-acting DNA elements which confer responsiveness on a promoter mediated through interaction with the DNA-binding domains of the first chimeric gene. This DNA element may be either palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon the nature of the cell or organism into which the response element will be incorporated. The DNA binding domain of the first hybrid protein binds, in the presence or absence of a ligand, to the DNA sequence of a response element to initiate or suppress transcription of downstream gene(s) under the regulation of this response element.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from a nucleic acid or polynucleotide. Expression may also refer to translation of mRNA into a protein or polypeptide.

The terms “cassette”, “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

The terms “modulate” and “modulates” mean to induce, reduce or inhibit nucleic acid or gene expression, resulting in the respective induction, reduction or inhibition of protein or polypeptide production.

The plasmids or vectors according to the invention may further comprise at least one promoter suitable for driving expression of a gene in a host cell. The term “expression vector” means a vector, plasmid or vehicle designed to enable the expression of an inserted nucleic acid sequence following transformation into the host. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving these genes is suitable for the present invention including but not limited to: viral promoters, bacterial promoters, animal promoters, mammalian promoters, synthetic promoters, constitutive promoters, tissue specific promoter, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TPI, alkaline phosphatase promoters (useful for expression in Saccharomyces); AOX1 promoter (useful for expression in Pichia); b-lactamase, lac, ara, tet, trp, lP_{L}, lP_{R}, T7, tac, and trc promoters (useful for expression in Escherichia coli); light regulated-, seed specific-, pollen specific-, ovary specific-, pathogenesis or disease related-, cauliflower mosaic virus 35S, CMV 35S minimal, cassava vein mosaic virus (CsVMV), chlorophyll a/b binding protein, ribulose 1, 5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art include, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus IE1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo AII control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell α-actin, and the like. In addition, these expression sequences may be modified by addition of enhancer or regulatory sequences and the like.

Enhancers that may be used in embodiments of the invention include but are not limited to: an SV40 enhancer, a cytomegalovirus (CMV) enhancer, an elongation factor 1 (EF1) enhancer, yeast enhancers, viral gene enhancers, and the like.

Termination control regions, i.e., terminator or polyadenylation sequences, may also be derived from various genes native to the preferred hosts. Optionally, a termination site may be unnecessary, however, it is most preferred if included. In certain embodiments of the invention, the termination control region may be comprised or be derived from a synthetic sequence, synthetic polyadenylation signal, an SV40 late polyadenylation signal, an SV40 polyadenylation signal, a bovine growth hormone (BGH) polyadenylation signal, viral terminator sequences, or the like.

The terms “3′ non-coding sequences” or “3′ untranslated region (UTR)” refer to DNA sequences located downstream (3′) of a coding sequence and may comprise polyadenylation [poly(A)] recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Regulatory region” means a nucleic acid sequence that regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include, without limitation, origins of replication, RNA splice sites, promoters, enhancers, transcriptional termination sequences, and signal sequences which direct the polypeptide into the secretory pathways of the target cell.

A regulatory region from a “heterologous source” is a regulatory region that is not naturally associated with the expressed nucleic acid. Included among the heterologous regulatory regions are, without limitation, regulatory regions from a different species, regulatory regions from a different gene, hybrid regulatory sequences, and regulatory sequences which do not occur in nature, but which are designed by one having ordinary skill in the art.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene. The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

A “polypeptide” is a polymeric compound comprised of covalently linked amino acid residues. Amino acids have the following general structure:

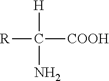

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the invention preferably comprises at least about 14 amino acids.

An “isolated polypeptide” or “isolated protein” is a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

A “fragment” of a polypeptide according to the invention will be understood to mean a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide according to the invention may have a length of at least 2-300 amino acids.

A “heterologous protein” refers to a protein not naturally produced in the cell.

A “mature protein” refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

The term “signal peptide” refers to an amino terminal polypeptide preceding the secreted mature protein. The signal peptide is cleaved from and is therefore not present in the mature protein. Signal peptides have the function of directing and translocating secreted proteins across cell membranes. Signal peptide is also referred to as signal protein.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667.). Such proteins (and their encoding genes) have sequence homology, as reflected by their high degree of sequence similarity. However, in common usage and in the instant application, the term “homologous,” when modified with an adverb such as “highly,” may refer to sequence similarity and not a common evolutionary origin.

Accordingly, the term “sequence similarity” in all its grammatical forms refers to the degree of identity or correspondence between nucleic acid or amino acid sequences of proteins that may or may not share a common evolutionary origin (see Reeck et al., 1987, Cell 50:667).

In a specific embodiment, two DNA sequences are “substantially homologous” or “substantially similar” when at least about 50% (preferably at least about 75%, and most preferably at least about 90 or 95%) of the nucleotides match over the defined length of the DNA sequences.

Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Sambrook et al., 1989, supra.

As used herein, “substantially similar” refers to nucleic acid fragments wherein changes in one or more nucleotide bases results in substitution of one or more amino acids, but do not affect the functional properties of the protein encoded by the DNA sequence. “Substantially similar” also refers to nucleic acid fragments wherein changes in one or more nucleotide bases does not affect the ability of the nucleic acid fragment to mediate alteration of gene expression by antisense or co-suppression technology. “Substantially similar” also refers to modifications of the nucleic acid fragments of the instant invention such as deletion or insertion of one or more nucleotide bases that do not substantially affect the functional properties of the resulting transcript. It is therefore understood that the invention encompasses more than the specific exemplary sequences. Each of the proposed modifications is well within the routine skill in the art, as is determination of retention of biological activity of the encoded products.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

A “substantial portion” of an amino acid or nucleotide sequence comprises enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method may be selected: KTUPLE 1, GAP PENALTY=3, WINDOW=5 and DIAGONALS SAVED=5.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available. Alternatively, or in addition to optimization to reflect codon bias, optimization can also include optimization of nucleotide sequence based on specific host cells wherein optimization is performed to maximize transcription rate or quantity, transcript half-life, and translation rate or quantity. Such optimization can be performed through empirical determinations based on specific host cell.

The term “gene switch” refers to the combination of a response element associated with a promoter, and a ligand-dependent transcription factor-based system which, in the presence of one or more ligands, modulates the expression of a gene with which the response element and promoter are operably associated. The term “a polynucleotide encoding a gene switch” refers to the combination of a response element associated with a promoter, and a polynucleotide encoding a ligand-dependent transcription factor-based system which, in the presence of one or more ligands, modulates the expression of a gene with which the response element and promoter are operably associated.

The terms “IL-12 activity” and “IL-12 biological activity” refer to any of the well-known bioactivities of IL-12, and include, without limitation, stimulating differentiation of naive T cells into Th1 cells, stimulating growth and function of T cells, stimulating production of interferon-gamma (IFN-gamma) and tumor necrosis factor-alpha (TNF-alpha) from T-cells and natural killer (NK) cells, stimulating reduction of IL-4 mediated suppression of IFN-gamma, stimulating enhancement of the cytotoxic activity of NK cells and CD8⁺ cytotoxic T lymphocytes, stimulating expression of IL-12R-beta1 and IL-12R-beta2, facilitating the presentation of tumor antigens through the upregulation of MHC I and II molecules, and stimulating anti-angiogenic activity. Exemplary assays for IL-12 activity include the Gamma Interferon Induction Assay (see Example 3, and U.S. Pat. No. 5,457,038). Additional assays are known in the art, such as, but not limited to, NK Cell Spontaneous Cytotoxicity Assays, ADCC Assays, Co-Mitogenic Effect Assays, and GM-CSF Induction Assays (e.g., as disclosed in Example 8 of U.S. Pat. No. 5,457,038, incorporated herein by reference).

In a preferred embodiment, IL-12 and scIL-12 polypeptides of the invention retain at least one IL-12 biological activity. In certain embodiments, IL-12 and scIL-12 polypeptides of the invention retain more than one IL-12 biological activity. In certain embodiments, IL-12 and scIL-12 polypeptides of the invention retain at least one, at least two, at least three, at least four, at least five or at least six of the above-referenced IL-12 biological activities. In certain embodiments, the IL-12 biological activity of IL-12 and scIL-12 polypeptides of the present invention is compared to (assayed against) the heterodimeric p35/p40 (wild-type) form of IL-12. In certain embodiments, IL-12 and scIL-12 polypeptides of the invention retain at least about 50%, at least about 75%, at least about 85%, at least about 90%, at least about 100%, at least 50%, at least 75%, at least 85%, at least 90%, at least 100%, or more of the biological activity of IL-12 compared to the heterodimeric p35/p40 (wild-type) form of IL-12. In one embodiment, IL-12 and scIL-12 polypeptides are modified to comprise proteolytic amino acid sequences, thereby rendering the biologically active composition susceptible to reduced in vivo (plasma) half-life.

As used herein, the terms “treating” or “treatment” of a disease refer to executing a protocol, which may include administering one or more drugs or in vitro engineered cells to a mammal (human or non-human), in an effort to alleviate signs or symptoms of the disease. Thus, “treating” or “treatment” should not necessarily be construed to require complete alleviation of signs or symptoms, does not require a cure, and specifically includes protocols that have only marginal effect on the subject.

As used herein, “immune cells” include dendritic cells, macrophages, neutrophils, mast cells, eosinophils, basophils, natural killer cells and lymphocytes (e.g., B and T cells).

As used herein, the term “stem cells” includes embryonic stem cells, adult stem cells and induced pluripotent stem cells. Stem cells can be obtained from any appropriate source, including bone marrow, adipose tissue, and blood (including, but not limited to, umbilical cord blood and menstrual blood). Examples of stem cells include, but are not limited to, mesenchymal stem cells and hematopoietic stem cells.

As used herein, the terms “dendritic cells” and “DC” are interchangeably used. Likewise, the terms “Natural Killer Cells” and “NK cells” are interchangeably used.

Polynucleotides Encoding Topologically Manipulated Single Chain IL-12 (“Topo scIL-12”) Polypeptides

The present invention includes polynucleotides encoding topologically manipulated single chain interleukin-12 (topo scIL-12) polypeptides, including full length and mature topo scIL-12 polypeptides wherein the sequences are (optionally) modified to comprise one or more amino acid substitutions that increase susceptibility of the polypeptide to proteolysis and/or reduce IL-12 biological half-life.

In accordance with specific embodiments of the present invention, nucleic acid sequences encoding modified topo scIL-12 polypeptides are provided. Specifically, the invention provides polynucleotides encoding a modified topo scIL-12 polypeptide comprising, from N- to C-terminus:

(i) a first IL-12 p40 domain (p40N),

(ii) an optional first peptide linker,

(iii) an IL-12 p35 domain,

(iv) an optional second peptide linker, and

(v) a second IL-12 p40 domain (p40C).

In one embodiment, topo scIL-12 polypeptides are modified to comprise proteolytic amino acid sequences. In one embodiment, modified topo scIL-12 polypeptides exhibit increased susceptibility to degradation (proteolysis) by proteinases (proteases) compared to corresponding unmodified topo scIL-12 polypeptides. In one embodiment, modified topo sclL-12 polypeptides have a reduced in vivo (e.g., plasma) half-life compared to corresponding unmodified topo scIL-12 polypeptides.

In certain embodiments, the first topo scIL-12 p40 domain (also referred to herein as p40N) encoded by polynucleotides of the invention is an N-terminal fragment of an IL-12 p40 subunit. IL-12 p40 polynucleotides for use in the invention include the human IL-12 p40 nucleic acid sequence of SEQ ID NO: 1 and the murine IL-12 p40 nucleic acid sequence of SEQ ID NO: 5, wherein the sequence is (optionally) further modified to encode one or more amino acid substitutions that increase susceptibility of the polypeptide to proteolysis and/or reduce IL-12 biological half-life. Additional, non-limiting examples of polynucleotides encoding IL-12 p40 subunits are available in public sequence databases, including but not limited to Genbank Accession Nos. AF180563.1 (human), NM_002187.2 (human), NG_009618.1 (human), NM_001077413.1 (cat), AF091134.1 (dog), NM_008352.2 (mouse), NM_001159424.1 (mouse), and NM_008351.2 (mouse), wherein the sequence is (optionally) further modified to encode one or more amino acid substitutions that increase susceptibility of the polypeptide to proteolysis and/or reduce IL-12 biological half-life.

N-terminal fragments of IL-12 p40 encoded by polynucleotides of the invention and suitable as a first topo sc IL-12 p40 domain (p40N) include, but are not limited to, polypeptides comprising, or alternatively consisting of, amino acids 1 to 288, 1 to 289, 1 to 290, 1 to 291, 1 to 292, 1 to 293, 1 to 294, 1 to 295, 1 to 296, 1 to 297, and 1 to 298 of SEQ ID NO: 2 wherein the sequence is (optionally) further modified to encode one or more amino acid substitutions that increase susceptibility of the polypeptide to proteolysis and/or reduce IL-12 biological half-life. A preferred N-terminal fragment of topo scIL-12 p40 encoded by polynucleotides of the invention and suitable as a first topo scIL-12 p40 domain (p40N) comprises, or alternatively consists of, amino acids 1 to 293 of SEQ ID NO: 2, wherein the sequence is (optionally) further modified to encode one or more amino acid substitutions that increase susceptibility of the polypeptide to proteolysis and/or reduce IL-12 biological half-life.

N-terminal fragments of topo scIL-12 p40 encoded by polynucleotides of the invention and suitable as a first topo scIL-12 p40 domain (p40N) may lack a signal sequence. It is understood that the specific cleavage site of a signal peptide may vary by 1, 2, 3 or more residues. Accordingly, in additional embodiments the first topo scIL-12 p40 domain (p40N) encoded by polynucleotides of the invention comprises, or alternatively consists of, a fragment of SEQ ID NO: 2 beginning with residue 18, 19, 20, 21, 22, 23, 24, 25, 26, 27 or 28 of SEQ ID NO: 2 and ending with residue 288, 289, 290, 291, 292, 293, 294, 295, 296, 297, or 298 of SEQ ID NO: 2 wherein the sequence is (optionally) further modified to encode one or more amino acid substitutions that increase susceptibility of the polypeptide to proteolysis and/or reduce IL-12 biological half-life. In one embodiment, a first topo scIL-12 p40 domain (p40N) encoded by polynucleotides of the invention comprises, or alternatively consists of, amino acid residues 23 to 293 of SEQ ID NO: 2 wherein the sequence is (optionally) further modified to encode one or more amino acid substitutions that increase susceptibility of the polypeptide to proteolysis and/or reduce IL-12 biological half-life.

The optional first peptide linker (ii) is any suitable peptide linker that allows folding of the topo scIL-12 polypeptide into a functional protein. In certain embodiments, the optional first topo scIL-12 peptide linker encoded by polynucleotides of the invention consists of 10 or fewer amino acids. In specific embodiments, the first topo scIL-12 peptide linker consists of 1, 2, 3, 4, 5, 6, 7, 8, 9, or 10 amino acids. In a preferred embodiment, the first topo scIL-12 peptide linker is selected from the peptides Thr-Pro-Ser (SEQ ID NO: 41) and Ser-Gly-Pro-Ala-Pro (SEQ ID NO: 42), and peptides with one amino acid substitution in Thr-Pro-Ser (SEQ ID NO: 41) and Ser-Gly-Pro-Ala-Pro (SEQ ID NO: 42). In certain embodiments the first topo scIL-12 peptide linker is absent. In some embodiments, any one or more linker sequences are modified to comprise one or more amino acid sequences that increase susceptibility of the linker to proteolysis and/or reduce IL-12 biological half-life.

In certain embodiments, the IL-12 p35 domain (iii) encoded by polynucleotides for use in the invention is a mature IL-12 p35 subunit, lacking a signal peptide. IL-12 p35 polynucleotides for use in the invention include the human IL-12 p35 nucleic acid sequence of SEQ ID NO: 3 and the murine IL-12 p35 nucleic acid sequence of SEQ ID NO: 7, wherein the sequence is (optionally) further modified to encode one or more amino acid substitutions that increase susceptibility of the polypeptide to proteolysis and/or reduce IL-12 biological half-life. Additional, non-limiting examples of polynucleotides encoding IL-12 p35 subunits are available in public sequence databases, including but not limited to AF101062.1 (human), NM_000882.3 (human), NG_033022.1 (human), NM_001159424.1 (mouse), NM_008351.2 (mouse), NM_001009833 (cat), NM_001082511.1 (horse), NM_001003293.1 (dog), wherein the sequence is (optionally) further modified to encode one or more amino acid substitutions that increase susceptibility of the polypeptide to proteolysis and/or reduce IL-12 biological half-life.

It is understood that the specific cleavage site of a signal peptide may vary by 1, 2, 3 or more residues. Accordingly, IL-12 p35 domains encoded by polynucleotides for use in the invention include the predicted mature sequence comprising, or alternatively consisting of, residues 57 to 253 of SEQ ID NO: 4 as well as mature sequences comprising, or alternatively consisting of, amino acids 52 to 253, 53 to 253, 54 to 253, 55 to 253, 56 to 253, 58 to 253, 59 to 253, 60 to 253, 61 to 263 and 62 to 253 of SEQ ID NO: 4, wherein the sequence is (optionally) further modified to comprise one or more amino acid substitutions that increase susceptibility of the polypeptide to proteolysis and/or reduce IL-12 biological half-life.

Suitable IL-12 p35 domains encoded by polynucleotides for use in the invention may be truncated at the C-terminus by one or more amino acid residues. Therefore, in additional embodiments the IL-12 p35 domain encoded by polynucleotides of the invention comprise, or alternatively consist of, a fragment of SEQ ID NO: 4 beginning with residue 52, 53, 54, 55, 56, 57, 58, 59, 60, or 61 of SEQ ID NO: 4 and ending with residue 247, 248, 249, 250, 251, 252, or 253 of SEQ ID NO: 4 wherein the sequence is (optionally) further modified to comprise one or more amino acid substitutions that increase susceptibility of the polypeptide to proteolysis and/or reduce IL-12 biological half-life.

The optional second peptide linker (iv) is any suitable peptide linker that allows folding of an scIL-12 polypeptide into a functional protein. In certain embodiments, the optional second peptide linker in topo scIL-12 encoded by polynucleotides of the invention consists of 10 or fewer amino acids. In specific embodiments, the second peptide linker in topo scIL-12 consists of 1, 2, 3, 4, 5, 6, 7, 8, 9, or 10 amino acids. In a preferred embodiment, the second peptide linker in topo scIL-12 is selected from the peptides Thr-Pro-Ser (SEQ ID NO: 41) and Ser-Gly-Pro-Ala-Pro (SEQ ID NO: 42), and peptides with one amino acid substitution in Thr-Pro-Ser (SEQ ID NO: 41) and Ser-Gly-Pro-Ala-Pro (SEQ ID NO: 42). In certain embodiments the second peptide linker in topo scIL-12 is absent. In a preferred embodiment, the first and second peptide linkers in topo scIL-12 consist of 10, 9, 8, 7 or fewer amino acid residues combined. In some embodiments any or all of the linkers are modified to comprise one or more amino acid sequences that increase susceptibility of the linker to proteolysis and/or reduction of IL-12 biological half-life

In certain embodiments, the second topo scIL-12 p40 domain (also referred to herein as p40C) encoded by polynucleotides of the invention is a C-terminal fragment of an IL-12 p40 subunit. C-terminal fragments of IL-12 p40 encoded by polynucleotides for use in the invention and suitable as a second IL-12 p40 domain (p40C) for use in the invention, comprise, or alternatively consist of, amino acids 289 to 328, 290 to 328, 291 to 328, 292 to 328, 293 to 328, 294 to 328, 295 to 328, 296 to 328, 297 to 328, 298 to 328, and 299 to 328 of SEQ ID NO: 2 wherein the sequence is (optionally) further modified to comprise one or more amino acid substitutions that increase susceptibility of the polypeptide to proteolysis and/or reduce IL-12 biological half-life.

Suitable second topo scIL-12 p40 domains (p40C) encoded by polynucleotides of the invention may be truncated at the C-terminus by one or more amino acid residues. Accordingly, in additional embodiments the second IL-12 p40 domain (p40C) encoded by polynucleotides for modification or not as part of the invention, comprise, or alternatively consist of, a fragment of SEQ ID NO: 2 beginning with residue 289, 290, 291, 292, 293, 294, 295, 296, 297, 298, or 299 of SEQ ID NO: 2 and ending with residue 322, 323, 324, 325, 326, 327, or 328 of SEQ ID NO: 2 wherein the sequence is (optionally) further modified to comprise one or more amino acid substitutions that increase susceptibility of the polypeptide to proteolysis and/or reduce IL-12 biological half-life.

The full-length sequence of a polynucleotide encoding a preferred scIL-12 polypeptide for use in the invention is presented herein as SEQ ID NO: 9 wherein the sequence is (optionally) further modified to encode one or more amino acid substitutions that increase susceptibility of the polypeptide to proteolysis and/or reduce IL-12 biological half-life. The full-length sequence encodes a predicted signal peptide at nucleic acids 1 to 66 of SEQ ID NO: 9, and a mature scIL-12 polypeptide at nucleic acids 67 to 1599 of SEQ ID NO: 9 wherein the sequence is (optionally) further modified to comprise one or more amino acid substitutions that increase susceptibility of the polypeptide to proteolysis and/or reduce IL-12 biological half-life.

Thus, a subject of the invention relates to an isolated polynucleotide encoding a modified scIL-12 polypeptide. In a specific embodiment, the isolated polynucleotide comprises a nucleic acid sequence selected from the group consisting of SEQ ID NO: 9 and nucleic acids 67 to 1599 of SEQ ID NO: 9 wherein the sequence is (optionally) further modified to encode one or more amino acid substitutions that increase susceptibility of the polypeptide to proteolysis and/or reduce IL-12 biological half-life. In a specific embodiment, the isolated polynucleotide further comprises a region permitting expression of the polypeptide in a host cell.

The present invention also relates to an isolated polynucleotide encoding a scIL-12 polypeptide comprising an amino acid sequence selected from the group consisting of SEQ ID NO: 10 and amino acids 23 to 533 of SEQ ID NO: 10 wherein the sequence is (optionally) further modified to encode one or more amino acid substitutions that increase susceptibility of the polypeptide to proteolysis and/or reduce IL-12 biological half-life.

The invention also provides polynucleotides encoding variants of the IL-12 polypeptides of the invention. In a preferred embodiment the polynucleotides of the invention encode a IL-12 variant polypeptide at least 80%, at least 85%, at least 90%, at least 95%, at least 97%, at least 98%, or at least 99% identical to the full-length or mature amino acid sequence of SEQ ID NO: 10, where the variant polypeptide exhibits at least one IL-12 activity, such as induction of IFN-gamma secretion from NK cells. Such IL-12 activities are readily determined using assays known in the art, such as the assays described in Example 8 of U.S. Pat. No. 5,457,038, which is incorporated herein by reference.

Due to the degeneracy of nucleotide coding sequences, other polynucleotides that encode substantially the same amino acid sequence as a IL-12 polynucleotide disclosed herein, including an amino acid sequence that contains a single amino acid variant, may be used in the practice of the present invention. These include but are not limited to allelic genes, homologous genes from other species, and nucleotide sequences comprising all or portions of a IL-12 polynucleotide that are altered by the substitution of different codons that encode the same amino acid residue within the sequence, thus producing a silent change. Likewise, the IL-12 derivatives of the invention include, but are not limited to, those comprising, as a primary amino acid sequence, all or part of the amino acid sequence of a IL-12 polypeptide including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations can be produced by various methods known in the art (see Sambrook et al., 1989, infra) and are not expected to affect apparent molecular weight as determined by polyacrylamide gel electrophoresis, or isoelectric point.

The present invention also relates to an isolated modified IL-12 polypeptide encoded by a polynucleotide according to the invention.

### Single Chain IL-12 Polypeptides

The present invention provides topologically manipulated (“topo”) scIL-12 polypeptides, including full length and mature topo scIL-12 polypeptides wherein the polypeptide has been further modified to encode one or more amino acid substitutions that increase susceptibility of the polypeptide to proteolysis and/or reduce IL-12 biological half-life.

Thus, the invention relates to isolated topo scIL-12 polypeptides. In a specific embodiment, the invention provides a scIL-12 polypeptide comprising, from N- to C-terminus:

(i) a first IL-12 p40 domain (p40N),

(ii) an optional first peptide linker,

(iii) an IL-12 p35 domain,

(iv) an optional second peptide linker, and

(v) a second IL-12 p40 domain (p40C)

wherein the sequence has been further modified to comprise one or more amino acid substitutions that increase susceptibility of the polypeptide to proteolysis and/or reduce IL-12 biological half-life.

In certain embodiments, the first topo scIL-12 p40 domain (p40N) is an N-terminal fragment of an IL-12 p40 subunit. IL-12 p40 polypeptides for use in the invention include the human IL-12 p40 amino acid sequence of SEQ ID NO: 2 and the murine IL-12 p40 amino acid sequence of SEQ ID NO: 6 wherein the sequence is (optionally) further modified to comprise one or more amino acid substitutions that increase susceptibility of the polypeptide to proteolysis and/or reduce IL-12 biological half-life. Additional, non-limiting examples of IL-12 p40 subunits which are further modified to encode one or more amino acid substitutions that increase susceptibility of the polypeptide to proteolysis and/or reduce IL-12 biological half-life are available in public sequence databases, including but not limited to Genbank Accession Nos. P29460.1 (human), AAD56386.1 (human), NP 005526.1 (human), NP 714912.1 (human), Q28268.1 (dog), NP_001003292.1 (dog), NP_032378.1 (mouse), NP_001152896.1 (mouse), NP_032377.1 (mouse).

N-terminal fragments of IL-12 p40 suitable as a first topo scIL-12 p40 domain (p40N) include, but are not limited to, polypeptides comprising, or alternatively consisting of, amino acids 1 to 288, 1 to 289, 1 to 290, 1 to 291, 1 to 292, 1 to 293, 1 to 294, 1 to 295, 1 to 296, 1 to 297, and 1 to 298 of SEQ ID NO: 2 wherein the sequence is (optionally) further modified to comprise one or more amino acid substitutions that increase susceptibility of the polypeptide to proteolysis and/or reduce IL-12 biological half-life. A preferred first topo scIL-12 p40 domain (p40N) comprises, or alternatively consists of, amino acids 1 to 293 of SEQ ID NO: 2 wherein the sequence is (optionally) further modified to comprise one or more amino acid substitutions that increase susceptibility of the polypeptide to proteolysis and/or reduce IL-12 biological half-life.

N-terminal fragments of IL-12 p40 suitable as a first topo scIL-12 p40 domain (p40N) may lack a signal sequence. Therefore, in additional embodiments the first topo sc IL-12 p40 domain (p40N) comprises, or alternatively consists of, a fragment of SEQ ID NO: 2 beginning with residue 18, 19, 20, 21, 22, 23, 24, 25, 26, 27 or 28 of SEQ ID NO: 2 and ending with residue 288, 289, 290, 291, 292, 293, 294, 295, 296, 297, or 298 wherein the sequence is (optionally) further modified to comprise one or more amino acid substitutions that increase susceptibility of the polypeptide to proteolysis and/or reduce IL-12 biological half-life. In one embodiment, the first IL-12 p40 domain (p40N) comprises, or alternatively consists of, amino acid residues 23 to 293 of SEQ ID NO: 2, wherein the sequence is (optionally) further modified to comprise one or more amino acid substitutions that increase susceptibility of the polypeptide to proteolysis and/or reduce IL-12 biological half-life.

The optional first peptide linker (ii) is any suitable peptide linker that allows folding of the topo scIL-12 polypeptide into a functional protein. In certain embodiments, the optional first topo scIL-12 peptide linker consists of 10 or fewer amino acids. In specific embodiments, the first topo scIL-12 peptide linker consists of 1, 2, 3, 4, 5, 6, 7, 8, 9, or 10 amino acids. In a preferred embodiment, the first topo scIL-12 peptide linker is selected from the peptides Thr-Pro-Ser (SEQ ID NO: 41) and Ser-Gly-Pro-Ala-Pro (SEQ ID NO: 42), and peptides with one amino acid substitution in Thr-Pro-Ser (SEQ ID NO: 41) and Ser-Gly-Pro-Ala-Pro (SEQ ID NO: 42). In certain embodiments the first topo scIL-12 peptide linker is absent. In certain embodiments a topo scIL-12 linker comprises an amino acid sequence that increases susceptibility of the polypeptide to proteolysis and/or reduced IL-12 biological half-life.

In certain embodiments, the IL-12 p35 domain (iii) is a mature IL-12 p35 subunit, lacking a signal peptide. IL-12 p35 polypeptides for use in the invention include the human IL-12 p35 amino acid sequence of SEQ ID NO: 4 and the murine IL-12p35 amino acid sequence of SEQ ID NO: 8, wherein the sequence is (optionally) further modified to comprise one or more amino acid substitutions that increase susceptibility of the polypeptide to proteolysis and/or reduce IL-12 biological half-life. Additional, non-limiting examples of IL-12 p35 subunits are available in public sequence databases, including but not limited to Genbank Accession Nos. AAB32758.1 (cat), NP_001003293 (dog), NP_001075980.1 (horse), NP_000873.2 (human), AAD56385.1 (human), NP_001152896.1 (mouse), and NP_032377.1 (mouse), wherein the sequence is (optionally) further modified to comprise one or more amino acid substitutions that increase susceptibility of the polypeptide to proteolysis and/or reduce IL-12 biological half-life.

It is understood that the specific cleavage site of a signal peptide may vary by 1, 2, 3 or more residues. Accordingly, in certain embodiments, mature p35 polypeptides of the invention include the predicted mature sequence consisting of residues 57 to 253 of SEQ ID NO: 4 as well as mature sequences consisting of amino acids 52 to 253, 53 to 253, 54 to 253, 55 to 253, 56 to 253, 58 to 253, 59 to 253, 60 to 253, 61 to 263 and 62 to 253 of SEQ ID NO: 4, wherein the sequence is (optionally) further modified to comprise one or more amino acid substitutions that increase susceptibility of the polypeptide to proteolysis and/or reduce IL-12 biological half-life.

Suitable IL-12 p35 domains may be truncated at the C-terminus by one or more amino acid residues. Therefore, in additional embodiments the IL-12 p35 domain comprises, or alternatively consists of, a fragment of SEQ ID NO: 4 beginning with residue 52, 53, 54, 55, 56, 57, 58, 59, 60, or 61 of SEQ ID NO: 4 and ending with residue 247, 248, 249, 250, 251, 252, or 253 of SEQ ID NO: 4, wherein the sequence is (optionally) further modified to comprise one or more amino acid substitutions that increase susceptibility of the polypeptide to proteolysis and/or reduce IL-12 biological half-life.

The optional second peptide linker (iv) is any suitable peptide linker that allows folding of the topo scIL-12 polypeptide into a functional protein. In certain embodiments, the optional second topo scIL-12 peptide linker consists of 10 or fewer amino acids. In specific embodiments, the second topo scIL-12 peptide linker consists of 1, 2, 3, 4, 5, 6, 7, 8, 9, or 10 amino acids. In preferred embodiments, the second topo scIL-12 peptide linker is selected from the peptides Thr-Pro-Ser (SEQ ID NO: 41) and Ser-Gly-Pro-Ala-Pro (SEQ ID NO: 42), and peptides with one amino acid substitution in Thr-Pro-Ser (SEQ ID NO: 41) and Ser-Gly-Pro-Ala-Pro (SEQ ID NO: 42). In certain embodiments the second topo scIL-12 peptide linker is absent. In a preferred embodiment, the first and second topo scIL-12 peptide linkers consist of 10 or fewer amino acid residues combined. In certain embodiments one or more topo scIL-12 peptide linkers comprise one or more amino acid sequences that increase susceptibility of the polypeptide to proteolysis and/or reduce IL-12 biological half-life.

In certain embodiments, the second IL-12 p40 domain (p40C) is a C-terminal fragment of an IL-12 p40 subunit. C-terminal fragments of p40 suitable as a second IL-12 p40 domain (p40C) comprise, or alternatively consist of, amino acids 289 to 328, 290 to 329, 291 to 328, 292 to 328, 293 to 328, 294 to 328, 295 to 328, 296 to 328, 297 to 328, 298 to 328, and 299 to 328 of SEQ ID NO: 2, wherein the sequence is (optionally) further modified to comprise one or more amino acid substitutions that increase susceptibility of the polypeptide to proteolysis and/or reduce IL-12 biological half-life.

Suitable second IL-12 p40 domains (p40C) may be truncated at the C-terminus by one or more amino acid residues. Therefore, in additional embodiments the second IL-12 p40 domain (p40C) comprises, or alternatively consists of, a fragment of SEQ ID NO: 2 beginning with residue 289, 290, 291, 292, 293, 294, 295, 296, 297, 298, or 299 of SEQ ID NO: 2 and ending with residue 322, 323, 324, 325, 326, 327, or 328 of SEQ ID NO: 2, wherein the sequence is (optionally) further modified to comprise one or more amino acid substitutions that increase susceptibility of the polypeptide to proteolysis and/or reduce IL-12 biological half-life.

The full-length sequence of a representative scIL-12 polypeptide of the invention is presented herein as SEQ ID NO: 10. The full-length sequence contains a predicted signal peptide at amino acids 1 to 22 of SEQ ID NO: 10, and a mature scIL-12 polypeptide at amino acids 23 to 533 of SEQ ID NO: 10, wherein the sequence is further modified to comprise one or more amino acid substitutions that increase susceptibility of the polypeptide to proteolysis and/or reduce IL-12 biological half-life.

In another specific embodiment, the scIL-12 polypeptide is encoded by a polynucleotide comprising a nucleic acid sequence selected from the group consisting of SEQ ID NO: 9 and nucleotides 67 to 1599 of SEQ ID NO: 9, wherein the sequence is further modified to encode one or more amino acid substitutions that increase susceptibility of the polypeptide to proteolysis and/or reduce IL-12 biological half-life.

Thus, a first subject of the invention relates to an isolated scIL-12 polypeptide. In a specific embodiment, the isolated polypeptide comprises an amino acid sequence selected from the group consisting of SEQ ID NO: 10 and amino acids 23 to 533 of SEQ ID NO: 10, wherein the sequence is further modified to comprise one or more amino acid substitutions that increase susceptibility of the polypeptide to proteolysis and/or reduce IL-12 biological half-life.

One of skill in the art is able to produce other polynucleotides to encode the polypeptides of the invention, by making use of the present invention and the degeneracy or non-universality of the genetic code as described herein.

Additional embodiments of the present invention include functional fragments of a topo scIL-12 polypeptide, or fusion proteins comprising a topo scIL-12 polypeptide of the present invention fused to second polypeptide comprising a heterologous, or normally non-contiguous, protein domain, wherein the sequence is further modified to comprise one or more amino acid substitutions that increase susceptibility of the polypeptide to proteolysis and/or reduce IL-12 biological half-life. Preferably, the second polypeptide is a targeting polypeptide such as an antibody, including single chain antibodies or antibody fragments. Thus, the invention provides a scIL-12 polypeptide fused at its N- or C-terminus to a second polypeptide, preferably to an antibody, an antibody fragment, or a single chain antibody, wherein the sequence is further modified to comprise one or more amino acid substitutions that increase susceptibility of the polypeptide to proteolysis and/or reduce IL-12 biological half-life.

The invention also provides variants of the topo scIL-12 polypeptides of the invention. In certain embodiments a topo scIL-12 variant polypeptide is at least 80%, at least 85%, at least 90%, at or at least 95%, at least 97%, at least 98%, or at least 99% identical to the full-length or mature amino acid sequence of SEQ ID NO: 10, where the variant polypeptide exhibits at least one IL-12 activity, such as induction of IFN-gamma secretion from NK cells. Such IL-12 activities are readily determined using assays known in the art, such as the assays described in Example 8 of U.S. Pat. No. 5,457,038, which is incorporated herein by reference.

The present invention also relates to compositions comprising an isolated polypeptide according to the invention.

The present invention relates to biologically active forms of an IL-12 complex (i.e., comprising p40 and p35 amino acid sequences (in either single chain or heterodimeric form)) wherein polypeptides forming the IL-12 complex have been modified to increase susceptibility to proteinases (proteases) to reduce the biologically active half-life of the IL-12 complex compared to a corresponding IL-12 complex lacking the proteinase susceptibility modifications.

In one example, an IL-12 p40 polypeptide is modified (e.g., genetically, synthetically or recombinantly engineered) to comprise non-naturally occurring regions of proteolytic susceptibility. Table 2 provides some examples of amino acid substitutions which are introduced into the IL-12 p40 polypeptide to increase susceptibility to proteolytic cleavage by matrix metalloproteinase-2 (MMP-2). Table 3 provides some examples of amino acid substitutions which are introduced into the IL-12 p40 polypeptide to increase susceptibility to proteolytic cleavage by plasmin. Table 4 provides some examples of amino acid substitutions which are introduced into the IL-12 p40 polypeptide to increase susceptibility to proteolytic cleavage by thrombin. Table 5 provides some examples of amino acid substitutions which are introduced into the IL-12 p40 polypeptide to increase susceptibility to proteolytic cleavage by urokinase-type plasminogen activator (uPA).

The amino acid substitutions examples indicated in Tables 2-5 are exemplified using the amino acid numbering of p40 in SEQ ID NO: 2 (which includes a predicted 22 amino acid signal peptide sequence). It is understood by those skilled in the art of the present invention that amino acid numbering in polypeptide sequences may differ depending on differences which may occur in signal peptide sequence cleavage (in vitro or in vivo) and depending on naturally occurring sequence variations among IL-12 p40 species. Those skilled in the art of the present invention understand that corresponding topological amino acid positions, when compared to the examples in Tables 2-5, may be used instead in IL-12 p40 sequences with variances in comparison to the amino acid numbering of SEQ ID NO: 2. (These Tables indicate amino acid name according to standard single letter code. The first letter represents the amino acid naturally occurring at the amino acid position indicated by the number immediately following. The second letter, following the amino acid position number, represents the amino acid residue to be substituted into that position. Forward slashes (“/”) in the Tables are indicative of the word “and”).

TABLE 2
Examples of amino acid substitutions in IL-12 p40 (SEQ ID NO: 2)
for increased susceptibility to proteolytic cleavage by MMP-2.

K126L
K124G/K126L
K124A/K126L
K124S/K126L
K124G/N125G/K126L
K124A/N125A/K126L
M45L
N248L
K247A/N248L
L246A/K247A/N248L
L246S/K247A/N248L
A172P
A172P/T174A
D40A/P42L
G161P/D164L

TABLE 3
Examples of amino acid substitutions in IL-12 p40 (SEQ ID NO: 2)
for increased susceptibility to proteolytic cleavage by plasmin.

D287S
K302S/N303S
V180S

TABLE 4
Examples of amino acid substitutions in IL-12 p40 (SEQ ID NO: 2)
for increased susceptibility to proteolytic cleavage by thrombin.

K280L/S281V/K282P/E284G/K285S
S176L/A177V/E178P/V180T/R181S
K280L/S281V/K282P/E284G/K285V
S176L/A177V/E178P/V180S/R181S

TABLE 5
Examples of amino acid substitutions in IL-12 p40 (SEQ ID NO: 2)
for increased susceptibility to proteolytic cleavage by uPA.

N248S/S249G
K282G/K285V
S249G
K282G/K307V

In another example, an IL-12 p35 polypeptide is modified (e.g., genetically, synthetically or recombinantly engineered) to comprise non-naturally occurring regions of proteolytic susceptibility. Table 6 provides some examples of amino acid substitutions which are introduced into the IL-12 p35 polypeptide to increase susceptibility to proteolytic cleavage by matrix metalloproteinase-2 (MMP-2). Table 7 provides some examples of amino acid substitutions which are introduced into the IL-12 p35 polypeptide to increase susceptibility to proteolytic cleavage by plasmin. Table 8 provides some examples of amino acid substitutions which are introduced into the IL-12 p35 polypeptide to increase susceptibility to proteolytic cleavage by thrombin. Table 9 provides some examples of amino acid substitutions which are introduced into the IL-12 p35 polypeptide to increase susceptibility to proteolytic cleavage by urokinase-type plasminogen activator (uPA).

The amino acid substitutions examples indicated in Tables 6-9 are exemplified using the amino acid numbering of p35 in SEQ ID NO: 4 (which includes a predicted 56 amino acid signal peptide sequence). It is understood by those skilled in the art of the present invention that amino acid numbering in polypeptide sequences may differ depending on differences which may occur in signal peptide sequence cleavage (in vitro or in vivo) and depending on naturally occurring sequence variations among IL-12 p35 species. Those skilled in the art of the present invention understand that corresponding topological amino acid positions, when compared to the examples in Tables 6-9, may be used instead in IL-12 p35 sequences with variances in comparison to the amino acid numbering of SEQ ID NO: 4. (These Tables indicate amino acid name according to standard single letter code. The first letter represents the amino acid naturally occurring at the amino acid position indicated by the number immediately following. The second letter, following the amino acid position number, represents the amino acid residue to be substituted into that position. Forward slashes (“/”) in the Tables are indicative of the word “and”).

TABLE 6
Examples of amino acid substitutions in IL-12 p35 (SEQ ID NO: 4)
for increased susceptibility to proteolytic cleavage by MMP-2.

Q186L
S215L
Y223L
K214P
K214P/S216A
C144P/S147L
C144P/L145S/S147L

TABLE 7
Examples of amino acid substitutions in IL-12 p35 (SEQ ID NO: 4)
for increased susceptibility to proteolytic cleavage by plasmin.

G142R/R148G
K149S
K149A
E135S
Q186S
S216R
D111A/K112R
Q213R/K214L/S215R/S216A

TABLE 8
Examples of amino acid substitutions in IL-12 p35 (SEQ ID NO: 4)
for increased susceptibility to proteolytic cleavage by thrombin.

A146V/S147P/K149G/T150S/S151K
N132V/S133P/E135G/T136S/S137K
S147P/K149I/T150I/S151K
N132F/S133P/E135G/S137K
N77I/L78P/S83R
T210L/Q213R/K214G

TABLE 9
Examples of amino acid substitutions in IL-12 p35 (SEQ ID NO: 4)
for increased susceptibility to proteolytic cleavage by uPA.

R148G/K149R
N207S/S208G/E209R
E209G/T210R

In another example, a topo scIL-12 polypeptide is modified (e.g., genetically, synthetically or recombinantly engineered) to introduce regions of proteolytic susceptibility. Table 10 provides some examples of amino acid substitutions which are introduced into the topo sc IL-12 polypeptide to increase susceptibility to proteolytic cleavage by matrix metalloproteinase-2 (MMP-2). Table 11 provides some examples of amino acid substitutions which are introduced into the topo sc IL-12 polypeptide to increase susceptibility to proteolytic cleavage by plasmin. Table 12 provides some examples of amino acid substitutions which are introduced into the topo sc IL-12 polypeptide to increase susceptibility to proteolytic cleavage by thrombin. Table 13 provides some examples of amino acid substitutions which are introduced into the topo sc IL-12 polypeptide to increase susceptibility to proteolytic cleavage by urokinase-type plasminogen activator (uPA).

The amino acid substitutions examples indicated in Tables 10-13 are exemplified using the amino acid numbering of topo sc IL-12 in SEQ ID NO: 10 (which includes a predicted 22 amino acid signal peptide sequence). It is understood by those skilled in the art of the present invention that amino acid numbering in polypeptide sequences may differ depending on differences which may occur in signal peptide sequence cleavage (in vitro or in vivo) and depending on other sequence variations which may be introduced among various topo sc IL-12 species. Those skilled in the art of the present invention understand that corresponding topological amino acid positions, when compared to the examples in Tables 10-13, may be used instead in topo sc IL-12 sequences with variances in comparison to the amino acid numbering of SEQ ID NO: 10. (These Tables indicate amino acid name according to standard single letter code. The first letter represents the amino acid naturally occurring at the amino acid position indicated by the number immediately following. The second letter, following the amino acid position number, represents the amino acid residue to be substituted into that position. Forward slashes (“/”) in the Tables are indicative of the word “and”).

TABLE 10
Examples of amino acid substitutions in topo sc IL-12 (SEQ ID NO:
10) for increased susceptibility to proteolytic cleavage by MMP-2.

K126L
K124G/K126L
K124A/K126L
K124S/K126L
K124G/N125G/K126L
K124A/N125A/K126L
M45L
N248L
K247A/N248L
L246A/K247A/N248L
L246S/K247A/N248L
Q426L
S455L
Y463L
A172P
A172P/T174A
K454P
K454P/S456A
C384P/S387L
C384P/L385S/S387L
D40A/P42L
G161P/D164L

TABLE 11
Examples of amino acid substitutions in topo sc IL-12 (SEQ ID NO: 10)
for increased susceptibility to proteolytic cleavage by plasmin.

D287S
K302S/N303S
V180S
G382R/R388G
K389S
K389A
E375S
Q426S
S456R
D351A/K352R
Q453R/K454L/S455R/S456A

TABLE 12
Examples of amino acid substitutions in topo sc IL-12 (SEQ ID NO: 10)
for increased susceptibility to proteolytic cleavage by thrombin.

K280L/S281V/K282P/E284G/K285S
S176L/A177V/E178P/V180T/R181S
A386V/S387P/K389G/T390S/S391K
N372V/S373P/E375G/T377S/S378K
K280L/S281V/K282P/E284G/K285V
S176L/A177V/E178P/V180S/R181S
S365P/K367I/T368I/S369K
N372F/S373P/E375G/S377K
N317I/L319P/S323R
T450L/Q453R/K454G

TABLE 13
Examples of amino acid substitutions in topo sc IL-12 (SEQ ID NO:
10) for increased susceptibility to proteolytic cleavage by uPA.

N248S/S249G
K282G/K285V
S249G
K282G/K285V
R388G/K389R
N447S/S448G/E449R
E449G/T450R

In certain embodiments modified IL-12 polypeptides of the invention comprise any combination of two or more sets of substitutions indicated in Tables 2-13. For example, in some embodiments a combination comprises any two, three, four, five, six, seven, eight, nine, ten, eleven, twelve or more sets of substitutions indicated in Tables 2-13.

In some embodiments, the proteolytic target sequence of the modified IL-12 polypeptides comprises a thrombin target sequence or a plasmin target sequence. In some embodiments, additional polypeptides are added to the C-terminus and/or N-terminus of the proteolytic target sequence. For example, 1, 2, 3, 4, 5, 6, 7, 8, 9, 10, 11, 12, 13, 14, 15, 16, 17, 18, 19, 20 or more than 20, e.g., 25, 30, 35, or 40, polypeptides can be added to the the C-terminus and/or N-terminus of the proteolytic target sequence. While not being bound by any theory, addition of polypeptides to the C-terminus and/or N-terminus of the proteolytic target sequence may allow for increased digestion of the proteolytic target sequence.

In some embodiments, the proteolytic target sequence comprises at least four polypeptides on N-terminal side of the proteolytic target sequence. In some embodiments, the proteolytic target sequence comprises at least four polypeptides on C-terminal side of the proteolytic target sequence. In some embodiments, the proteolytic target sequence comprises a at least four polypeptides on N-terminal side of the proteolytic target sequence and at least four polypeptides on C-terminal side of the proteolytic target sequence.

In some embodiments, the proteolytic target sequence comprises four to twenty polypeptides on N-terminal side of the proteolytic target sequence. In some embodiments, the proteolytic target sequence comprises four to twenty polypeptides on C-terminal side of the proteolytic target sequence. In some embodiments, the proteolytic target sequence comprises four to twenty polypeptides on N-terminal side of the proteolytic target sequence and four to twenty polypeptides on C-terminal side of the proteolytic target sequence.

In some embodiments, the proteolytic target sequence comprises a thrombin target sequence or a plasmin target sequence, and at least four polypeptides on N-terminal side of the thrombin target sequence or a plasmin target sequence. In some embodiments, the proteolytic target sequence comprises a thrombin target sequence or a plasmin target sequence, and at least four polypeptides on C-terminal side of the thrombin target sequence or a plasmin target sequence. In some embodiments, the proteolytic target sequence comprises a thrombin target sequence or a plasmin target sequence, at least four polypeptides on N-terminal side of the thrombin target sequence or a plasmin target sequence. and at least four polypeptides on C-terminal side of the thrombin target sequence or a plasmin target sequence.

In some embodiments, the proteolytic target sequence comprises a thrombin target sequence or a plasmin target sequence, and four to twenty polypeptides on N-terminal side of the thrombin target sequence or a plasmin target sequence. In some embodiments, the proteolytic target sequence comprises a thrombin target sequence or a plasmin target sequence, and four to twenty polypeptides on C-terminal side of the thrombin target sequence or a plasmin target sequence. In some embodiments, the proteolytic target sequence comprises a thrombin target sequence or a plasmin target sequence, four to twenty polypeptides on N-terminal side of the thrombin target sequence or a plasmin target sequence. and four to twenty polypeptides on C-terminal side of the thrombin target sequence or a plasmin target sequence. In some embodiments, the composition comprises a linker sequence selected from the group consisting of SEQ ID NOS 60, 68, 76, 67, 62, 65, 66, 75 and 72.

### Compositions

The present invention also relates to compositions comprising IL-12 polynucleotides or polypeptides according to the invention. Such compositions may comprise a IL-12 polypeptide or a polynucleotide encoding a IL-12 polypeptide, as defined above, and an acceptable carrier or vehicle. The compositions of the invention are particularly suitable for formulation of biological material for use in therapeutic administration. Thus, in one embodiment, the composition comprises a polynucleotide encoding a IL-12 polypeptide. In another embodiment, the composition comprises a IL-12 polypeptide according to the invention.

The phrase “acceptable” refers to molecular entities and compositions that are physiologically tolerable to the cell or organism when administered. The term “carrier” refers to a diluent, adjuvant, excipient, or vehicle with which the composition is administered. Such carriers can be sterile liquids, such as water and oils, including those of petroleum, animal, vegetable or synthetic origin, such as peanut oil, soybean oil, mineral oil, sesame oil and the like. Examples of acceptable carriers are saline, buffered saline, isotonic saline (e.g., monosodium or disodium phosphate, sodium, potassium, calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin. Pharmaceutical compositions of the invention may be formulated for the purpose of topical, oral, parenteral, intranasal, intravenous, intramuscular, intratumoral, subcutaneous, intraocular, and the like, administration.

Preferably, the compositions comprise an acceptable vehicle for an injectable formulation. This vehicle can be, in particular, a sterile, isotonic saline solution (monosodium or disodium phosphate, sodium, potassium, calcium or magnesium chloride, and the like, or mixtures of such salts), or dry, in particular lyophilized, compositions which, on addition, as appropriate, of sterilized water or of physiological saline, enable injectable solutions to be formed. The preferred sterile injectable preparations can be a solution or suspension in a nontoxic parenterally acceptable solvent or diluent.

In yet another embodiment, a composition comprising a modified IL-12 polypeptide, or polynucleotide encoding the polypeptide, can be delivered in a controlled release system. For example, the polynucleotide or polypeptide may be administered using intravenous infusion, an implantable osmotic pump, a transdermal patch, liposomes, or other modes of administration. Other controlled release systems are discussed in the review by Langer [Science 249:1527-1533 (1990)].

### Expression of IL-12 Polypeptides

With the sequence of the IL-12 polypeptides and the polynucleotides encoding them, large quantities of IL-12 polypeptides may be prepared. By the appropriate expression of vectors in cells, high efficiency production may be achieved. Thereafter, standard purification methods may be used, such as ammonium sulfate precipitations, column chromatography, electrophoresis, centrifugation, crystallization and others. See various volumes of Methods in Enzymology for techniques typically used for protein purification. Alternatively, in some embodiments high efficiency of production is unnecessary, but the presence of a known inducing protein within a carefully engineered expression system is quite valuable. Typically, the expression system will be a cell, but an in vitro expression system may also be constructed.

A polynucleotide encoding a IL-12, or fragment, derivative or analog thereof, or a functionally active derivative, including a chimeric protein, thereof, can be inserted into an appropriate expression vector, i.e., a vector which comprises the necessary elements for the transcription and translation of the inserted protein-coding sequence. A polynucleotide of the invention is operationally linked with a transcriptional control sequence in an expression vector. An expression vector also preferably includes a replication origin.

The isolated polynucleotides of the invention may be inserted into any appropriate cloning vector. A large number of vector-host systems known in the art may be used. Possible vectors include, but are not limited to, plasmids or modified viruses, but the vector system must be compatible with the host cell used. Examples of vectors include, but are not limited to, Escherichia coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the polynucleotide into a cloning vector that has complementary cohesive termini. However, if the complementary restriction sites used to fragment the polynucleotide are not present in the cloning vector, the ends of the polynucleotide molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Preferably, the cloned gene is contained on a shuttle vector plasmid, which provides for expansion in a cloning cell, e.g., E. coli, and purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2μ plasmid.

In addition, the present invention relates to an expression vector comprising a polynucleotide according the invention, operatively linked to a transcription regulatory element. In one embodiment, the polynucleotide is operatively linked with an expression control sequence permitting expression of the IL-12 polypeptide in an expression competent host cell. The expression control sequence may comprise a promoter that is functional in the host cell in which expression is desired. The vector may be a plasmid DNA molecule or a viral vector. In certain embodiments, viral vectors include, without limitation, retrovirus, adenovirus, adeno-associated virus (AAV), herpes virus, and vaccinia virus. The invention further relates to a replication defective recombinant virus comprising in its genome, a polynucleotide according to the invention. Thus, the present invention also relates to an isolated host cell comprising such an expression vector, wherein the transcription regulatory element is operative in the host cell.

The desired genes will be inserted into any of a wide selection of expression vectors. The selection of an appropriate vector and cell line depends upon the constraints of the desired product. Typical expression vectors are described in Sambrook et al. (1989). Suitable cell lines may be selected from a depository, such as the ATCC. See, ATCC Catalogue of Cell Lines and Hybridomas (6th ed.) (1988); ATCC Cell Lines, Viruses, and Antisera, each of which is hereby incorporated herein by reference. The vectors are introduced to the desired cells by standard transformation or transfection procedures as described, for instance, in Sambrook et al. (1989). Fusion proteins will typically be made by either recombinant nucleic acid methods or by synthetic polypeptide methods. Techniques for nucleic acid manipulation are described generally, for example, in Sambrook et al. (1989), Molecular Cloning: A Laboratory Manual (2d ed.), Vols. 1-3, Cold Spring Harbor Laboratory, which are incorporated herein by reference. Techniques for synthesis of polypeptides are described, for example, in Merrifield, J. Amer. Chem. Soc. 85:2149-2156 (1963).

Once a particular recombinant DNA molecule is identified and isolated, any of multiple methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus, adenovirus, or adeno-associated virus (AAV); insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Different host cells have characteristic and specific mechanisms for the translational and post-translational processing and modification of proteins. Appropriate cell lines or host systems can be chosen to ensure the desired modification and processing of the foreign protein expressed. Expression in yeast can produce a biologically active product. Expression in eukaryotic cells can increase the likelihood of “native” folding. Moreover, expression in mammalian cells can provide a tool for reconstituting, or constituting, IL-12 activity. Furthermore, different vector/host expression systems may affect processing reactions, such as proteolytic cleavages, to a different extent.

Vectors are introduced into the desired host cells by methods known in the art, e.g., transfection, electroporation, microinjection, transduction, cell fusion, DEAE dextran, calcium phosphate precipitation, lipofection (lysosome fusion), particle bombardment, use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

### Vectors and Gene Expression Cassettes Comprising IL-12 Polynucleotides

The present invention also relates to a vector comprising a polynucleotide encoding a IL-12 polypeptide according to the invention. The present invention also provides a gene expression cassette comprising a polynucleotide encoding a IL-12 polypeptide according to the invention. The polynucleotides of the invention, where appropriate incorporated in vectors or gene expression cassettes, and the compositions comprising them, are useful for enhancing immune system function, for example as vaccine adjuvants and in combination with other immunomodulators and/or small molecule pharmaceuticals in the treatment of infections and cancer. They may be used for the transfer and expression of genes in vitro or in vivo in any type of cell or tissue. The transformation can, moreover, be targeted (transfer to a particular tissue can, in particular, be determined by the choice of a vector, and expression by the choice of a particular promoter). The polynucleotides and vectors of the invention are advantageously used for the production in vivo of IL-12 polypeptides of the invention.

The polynucleotides encoding the IL-12 polypeptides of the invention may be used in a plasmid vector. Preferably, an expression control sequence is operably linked to the IL-12 polynucleotide coding sequence for expression of the IL-12 polypeptide. The expression control sequence may be any enhancer, response element, or promoter system in vectors capable of transforming or transfecting a host cell. Once the vector has been incorporated into the appropriate host, the host, depending on the use, will be maintained under conditions suitable for high level expression of the polynucleotides.

Polynucleotides will normally be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences (see, e.g. U.S. Pat. No. 4,704,362, which is incorporated herein by reference).

Escherichia coli is one prokaryotic host useful for cloning the polynucleotides of the present invention. Other microbial hosts suitable for use include, without limitation, bacilli, such as Bacillus subtilis, and other enterobacteriaceae, such as Salmonella, Serratia, and various Pseudomonas species.

Other eukaryotic cells may be used, including, without limitation, yeast cells, insect tissue culture cells, avian cells or the like. Preferably, mammalian tissue cell culture will be used to produce the polypeptides of the present invention (see, Winnacker, From Genes to Clones, VCH Publishers, N.Y. (1987), which is incorporated herein by reference).

Expression vectors may also include, without limitation, expression control sequences, such as an origin of replication, a promoter, an enhancer, a response element, and necessary processing information sites, such as ribosome-binding sites, RNA splice sites, polyadenylation sites, and transcriptional terminator sequences. Preferably, the enhancers or promoters will be those naturally associated with genes encoding the IL-12 subunits p40 and p35, although it will be understood that in many cases others will be equally or more appropriate. In further embodiments, expression control sequences are enhancers or promoters derived from viruses, such as SV40, Adenovirus, Bovine Papilloma Virus, and the like.

The vectors comprising the polynucleotides of the present invention can be transferred into the host cell by well-known methods, which vary depending on the type of cellular host. For example, calcium chloride transfection is commonly utilized for prokaryotic cells, whereas calcium phosphate treatment may be used for other cellular hosts. (See, generally, Sambrook et al. (1989), Molecular Cloning: A Laboratory Manual (2d ed.), Cold Spring Harbor Press, which is incorporated herein by reference.) The term “transformed cell” is meant to also include the progeny of a transformed cell.

Potential host-vector systems include but are not limited to mammalian cell systems infected with virus (e.g., vaccinia virus, adenovirus, adeno-associated virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant IL-12 protein of the invention, or functional fragment, derivative, chimeric construct, or analog thereof, may be expressed chromosomally, after integration of the coding sequence by recombination. In this regard, any of a number of amplification systems may be used to achieve high levels of stable gene expression (See Sambrook et al., 1989, supra).

The cell containing the recombinant vector comprising the IL-12 polynucleotide is cultured in an appropriate cell culture medium under conditions that provide for expression of the IL-12 polypeptide by the cell. Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

A polynucleotide encoding a IL-12 polypeptide may be operably linked and controlled by any regulatory region, i.e., promoter/enhancer element known in the art, but these regulatory elements must be functional in the host cell selected for expression. The regulatory regions may comprise a promoter region for functional transcription in the host cell, as well as a region situated 3′ of the gene of interest, and which specifies a signal for termination of transcription and a polyadenylation site. All these elements constitute an expression cassette.

Expression vectors comprising a polynucleotide encoding a IL-12 polypeptide of the invention can be identified by five general approaches: (a) PCR amplification of the desired plasmid DNA or specific mRNA, (b) nucleic acid hybridization, (c) presence or absence of selection marker gene functions, (d) analyses with appropriate restriction endonucleases, and (e) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding a IL-12 polypeptide is inserted within the “selection marker” gene sequence of the vector, recombinants comprising the IL-12 nucleic acid insert can be identified by the absence of the gene function. In the fourth approach, recombinant expression vectors are identified by digestion with appropriate restriction enzymes. In the fifth approach, recombinant expression vectors can be identified by assaying for the activity, biochemical, or immunological characteristics of the gene product expressed by the recombinant, provided that the expressed protein assumes a functionally active conformation.

A wide variety of host/expression vector combinations may be employed in expressing the DNA sequences of this invention. Useful expression vectors, for example, may consist of segments of chromosomal, non-chromosomal and synthetic DNA sequences. Suitable vectors include but are not limited to derivatives of SV40 and known bacterial plasmids, e.g., E. coli plasmids col E1, pCR1, pBR322, pMal-C2, pET, pGEX (Smith et al., 1988, Gene 67:31-40), pMB9 and their derivatives, plasmids such as RP4; phage DNAS, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

The present invention also provides a gene expression cassette that is capable of being expressed in a host cell, wherein the gene expression cassette comprises a polynucleotide that encodes a IL-12 polypeptide according to the invention. Thus, Applicants' invention also provides novel gene expression cassettes useful in a IL-12 expression system.

Gene expression cassettes of the invention may include a gene switch to allow the regulation of gene expression by addition or removal of a specific ligand. In one embodiment, the gene switch is one in which the level of gene expression is dependent on the level of ligand that is present. Examples of ligand-dependent transcription factor complexes that may be used in the gene switches of the invention include, without limitation, members of the nuclear receptor superfamily activated by their respective ligands glucocorticoid, estrogen, progestin, retinoid, ecdysone, and analogs and mimetics thereof); rTTA activated by tetracycline; Biotin-based switch systems; FKBP/rapamycin switch systems; cumate switch systems; riboswitch systems; among others.

In one aspect of the invention, the gene switch is an EcR-based gene switch. Examples of such systems include, without limitation, the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. application Ser. No. 10/468,193 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. application Ser. No. 14/001,943 (U.S. Pub. No. [Pending]), each of which is incorporated by reference in its entirety.

In another aspect of the invention, the gene switch is based on heterodimerization of FK506 binding protein (FKBP) with FKBP rapamycin associated protein (FRAP) and is regulated through rapamycin or its non-immunosuppressive analogs. Examples of such systems include, without limitation, the ARGENT′ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595.

In another aspect of the invention, gene expression cassettes of the invention incorporate a cumate switch system, which works through the CymR repressor that binds the cumate operator sequences with high affinity. (SparQ™ Cumate Switch, System Biosciences, Inc.) The repression is alleviated through the addition of cumate, a non-toxic small molecule that binds to CymR. This system has a dynamic inducibility, can be finely tuned and is reversible and inducible.

In another aspect of the invention, gene expression cassettes of the invention incorporate a riboswitch, which is a regulatory segment of a messenger RNA molecule that binds an effector, resulting in a change in production of the proteins encoded by the mRNA. An mRNA that contains a riboswitch is directly involved in regulating its own activity in response to the concentrations of its effector molecule. Effectors can be metabolites derived from purine/pyrimidine, amino acid, vitamin, or other small molecule co-factors. These effectors act as ligands for the riboswitch sensor, or aptamer. Breaker, R R. Mol Cell. (2011) 43(6):867-79.

In another aspect of the invention, gene expression cassettes of the invention incorporate the biotin-based gene switch system, in which the bacterial repressor protein TetR is fused to streptavidin, which interacts with the synthetic biotinylation signal AVITAG that is fused to VP16 to activate gene expression. Biotinylation of the AVITAG peptide is regulated by a bacterial biotin ligase BirA, thus enabling ligand responsiveness. Weber et al. (2007) Proc. Natl. Acad. Sci. U.S.A. 104, 2643-2648; Weber et al. (2009) Metabolic Engineering, 11(2):117-124.

Additional gene switch systems appropriate for use in the instant invention are well known in the art, including but not limited to those described in Auslander and Fussenegger, Trends in Biotechnology (2012), 31(3):155-168, incorporated herein by reference.

Examples of ligands for use in gene switch systems include, without limitation, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; arnidoketones such as those described in U.S. Published Application No. 2004/0049037; each of which is incorporated herein by reference and other similar materials including 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, and the like. Examples of diacylhydrazine ligands useful in the present invention include RG-115819 (3,5-Dimethyl-benzoic acid N-(1-ethyl-2,2-dimethyl-propyl)-N′-(2-methyl-3-methoxy-benzoyl)-hydrazide), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See, e.g., U.S. patent application Ser. No. 12/155,111, and PCT Appl. No. PCT/US2008/006757, both of which are incorporated herein by reference in their entireties.

### Antibodies to Modified IL-12 Polypeptides

According to the invention, a modified IL-12 polypeptide produced recombinantly or by chemical synthesis, and fragments or other derivatives or analogs thereof, including fusion proteins, may be used as an antigen or immunogen to generate antibodies. Preferably, the antibodies specifically bind modified IL-12 polypeptides, but do not bind non-modified IL-12 polypeptides. More preferably, the antibodies specifically bind a modified topo scIL-12 polypeptide, but do not bind other cytokine polypeptides.

In another embodiment, the invention relates to an antibody which specifically binds an antigenic peptide comprising a fragment of a modified IL-12 polypeptide according to the invention as described above. The antibody may be polyclonal or monoclonal and may be produced by in vitro or in vivo techniques.

The antibodies of the invention possess specificity for binding to particular modified IL-12 polypeptides. Thus, reagents for determining qualitative or quantitative presence of these or homologous polypeptides may be produced. Alternatively, these antibodies may be used to separate or purify modified IL-12 polypeptides.

For production of polyclonal antibodies, an appropriate target immune system is selected, typically a mouse or rabbit. The substantially purified antigen is presented to the immune system in a fashion determined by methods appropriate for the animal and other parameters well known to immunologists. Typical sites for injection are in the footpads, intramuscularly, intraperitoneally, or intradermally. Of course, another species may be substituted for a mouse or rabbit.

An immunological response is usually assayed with an immunoassay. Normally such immunoassays involve some purification of a source of antigen, for example, produced by the same cells and in the same fashion as the antigen was produced. The immunoassay may be a radioimmunoassay, an enzyme-linked assay (ELISA), a fluorescent assay, or any of many other choices, most of which are functionally equivalent but may exhibit advantages under specific conditions.

Monoclonal antibodies with high affinities are typically made by standard procedures as described, e.g., in Harlow and Lane (1988), Antibodies: A Laboratory Manual, Cold Spring Harbor Laboratory; or Goding (1986), Monoclonal Antibodies: Principles and Practice (2nd Ed.) Academic Press, New York, which are hereby incorporated herein by reference. Briefly, appropriate animals will be selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone are tested for their production of an appropriate antibody specific for the desired region of the antigen.

Other suitable techniques involve in vitro exposure of lymphocytes to the antigenic polypeptides or alternatively to selection of libraries of antibodies in phage or similar vectors. See, Huse et al., (1989) “Generation of a Large Combinatorial Library of the Immunoglobulin Repertoire in Phage Lambda,” Science 246:1275-1281, hereby incorporated herein by reference.

The polypeptides and antibodies of the present invention may be used with or without modification. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include, without limitation, radionuclides, enzymes, substrates, cofactors, inhibitors, fluorescence, chemiluminescence, magnetic particles and the like. Patents, teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly, U.S. Pat. No. 4,816,567.

A molecule is “antigenic” when it is capable of specifically interacting with an antigen recognition molecule of the immune system, such as an immunoglobulin (antibody) or T cell antigen receptor. An antigenic polypeptide contains at least about 5, and preferably at least about 10 amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

Such antibodies include but are not limited to polyclonal, monoclonal, chimeric, single chain, Fab fragments, and an Fab expression library. The modified IL-12 antibodies of the invention may be cross reactive, e.g., they may recognize modified IL-12 polypeptides derived from different species. Polyclonal antibodies have greater likelihood of cross reactivity. Alternatively, an antibody of the invention may be specific for a single form of modified IL-12 polypeptide, such as a modified human IL-12 polypeptide. Preferably, such an antibody is specific for modified human topo scIL-12.

Various procedures known in the art may be used for the production of polyclonal antibodies. For the production of antibody, various host animals can be immunized by injection with a modified IL-12 polypeptide, or a derivative (e.g., fragment or fusion protein) thereof, including but not limited to rabbits, mice, rats, sheep, goats, etc. In one embodiment, the modified IL-12 polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward a modified IL-12 polypeptide, or fragment, analog, or derivative thereof, any technique that provides for the production of antibody molecules by continuous cell lines in culture may be used. These include but are not limited to the hybridoma technique originally developed by Kohler and Milstein [Nature 256:495-497 (1975)], as well as the trioma technique, the human B-cell hybridoma technique [Kozbor et al., Immunology Today 4:72 1983); Cote et al., Proc. Natl. Acad. Sci. U.S.A. 80:2026-2030 (1983)1, and the EBV-hybridoma technique to produce human monoclonal antibodies [Cole et al., in Monoclonal Antibodies and Cancer Therapy, Alan R. Liss, Inc., pp. 77-96 (1985)]. In an additional embodiment of the invention, monoclonal antibodies can be produced in germ-free animals [International Patent Publication No. WO 89/12690, published 28 Dec. 1989]. In fact, according to the invention, techniques developed for the production of “chimeric antibodies” [Morrison et al., J. Bacteriol. 159:870 (1984); Neuberger et al., Nature 312:604-608 (1984); Takeda et al., Nature 314:452-454 (1985)] by splicing the genes from a mouse antibody molecule specific for a modified IL-12 polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain Fv (scFv) antibodies [U.S. Pat. Nos. 5,476,786 and 5,132,405 to Huston; U.S. Pat. No. 4,946,778] can be adapted to produce modified IL-12 polypeptide-specific single chain antibodies. An additional embodiment of the invention utilizes the techniques described for the construction of Fab expression libraries [Huse et al., Science 246:1275-1281 (1989)] to allow rapid and easy identification of monoclonal Fab fragments with the desired specificity for a modified IL-12 polypeptide, or its derivatives, or analogs.

Antibody fragments which contain the idiotype of the antibody molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots (immunoblots), precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a modified IL-12 polypeptide, one may assay generated hybridomas for a product which binds to a modified IL-12 polypeptide fragment containing such epitope.

The foregoing antibodies can be used in methods known in the art relating to the localization and activity of a modified IL-12 polypeptide, e.g., for western blotting, imaging a modified IL-12 polypeptide in situ, measuring levels thereof in appropriate physiological samples, etc. using any of the detection techniques mentioned above or known in the art.

### Uses of Modified IL-12 Polynucleotides and Polypeptides

The modified IL-12 polypeptides and polynucleotides of the present invention have a variety of utilities. For example, the polynucleotides and polypeptides of the invention are useful in the treatment of diseases in which stimulation of immune function might be beneficial. In specific embodiments, the modified IL-12 polypeptides and polynucleotides of the present invention are useful for the treatment of disease states responsive to the enhanced presence of gamma interferon; for the treatment of viral, bacterial, protozoan and parasitic infections; and for the treatment of proliferative disorders such as cancer. The modified IL-12 polynucleotides and polypeptides of the invention are also useful as vaccine adjuvants.

### Methods of Inducing IFN-Gamma Production

The modified IL-12 polypeptide and polynucleotide compositions of the invention are useful for inducing the production of IFN-gamma in a patient in need thereof. Pathological states which benefit from IFN-gamma induction may result from disease, exposure to radiation or drugs, and include for example but without limitation, leukopenia, bacterial and viral infections, anemia, B cell or T cell deficiencies including immune cell or hematopoietic cell deficiency following a bone marrow transplantation.

### Methods of Treating Infections

The modified IL-12 polypeptide and polynucleotide compositions according to the present invention can be used in the treatment of viral infections, including without limitation, HIV, Hepatitis A, Hepatitis B, Hepatitis C, rabies virus, poliovirus, influenza virus, meningitis virus, measles virus, mumps virus, rubella, pertussis, encephalitis virus, papilloma virus, yellow fever virus, respiratory syncytial virus, parvovirus, chikungunya virus, haemorrhagic fever viruses, Klebsiella, and Herpes viruses, particularly, varicella, cytomegalovirus and Epstein-Barr virus infection, among others.

The modified IL-12 polypeptide and polynucleotide compositions according to the present invention can be used in the treatment of bacterial infections, including, without limitation, leprosy, tuberculosis, Yersinia pestis, Typhoid fever, pneumococcal bacterial infections, tetanus and anthrax, among others.

The modified IL-12 polypeptide and polynucleotide compositions according to the present invention can also be used in the treatment of parasitic infections, such as, but not limited to, leishmaniasis and malaria, among others; and protozoan infections, such as, but not limited to, T. cruzii) or helminths, such as Schistosoma.

### Methods of Use as a Vaccine Adjuvant

The modified IL-12 polypeptide and polynucleotide compositions are useful as vaccine adjuvants. By “adjuvant” is meant a substance which enhances the immune response when administered together with an immunogen or antigen.

The modified IL-12 polypeptide and polynucleotide compositions of the invention are useful for enhancing the immune response to viral vaccines, including without limitation, HIV, Hepatitis A, Hepatitis B, Hepatitis C, rabies virus, poliovirus, influenza virus, meningitis virus, measles virus, mumps virus, rubella, pertussis, encephalitis virus, papilloma virus, yellow fever virus, respiratory syncytial virus, parvovirus, chikungunya virus, haemorrhagic fever viruses, Klebsiella, and Herpes viruses, particularly, varicella, cytomegalovirus and Epstein-Barr virus.

The modified IL-12 polypeptide and polynucleotide compositions of the invention are also useful for enhancing the immune response to bacterial vaccines, such as, but not limited to, vaccines against leprosy, tuberculosis, Yersinia pestis, Typhoid fever, pneumococcal bacteria, tetanus and anthrax, among others.

Similarly, polypeptides and polynucleotides of the invention are also useful for enhancing the immune response to vaccines against parasitic infections (such as leishmaniasis and malaria, among others) and vaccines against protozoan infections (e.g., T. cruzii) or helminths, e.g., Schistosoma.

The modified IL-12 polypeptide and polynucleotide compositions of the invention are also useful for enhancing the immune response to a therapeutic cancer vaccine. A cancer vaccine may comprise an antigen expressed on the surface of a cancer cell. This antigen may be naturally present on the cancer cell. Alternatively, the cancer cell may be manipulated ex vivo and transfected with a selected antigen, which it then expresses when introduced into the patient. A nonlimiting example of a cancer vaccine which may be enhanced by polynucleotides and polypeptides of the invention includes Sipuleucel-T (Provenge®).

Methods of formulating and administering vaccine adjuvants are known in the art, such as the methods described in U.S. Pat. No. 5,571,515, which are herein incorporated by reference.

### Methods of Treating Cancer

The modified IL-12 polypeptide and polynucleotide compositions according to the present invention can be used to treat a cancer. Non-limiting examples of cancers that can be treated according to the invention include without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, mesothelioma, osteogenic sarcoma, primary macroglobulinemia, and retinoblastoma, and the like.

The invention provides a method of treating cancer comprising administering a modified IL-12 polypeptide of the invention to a patient in a therapeutically effective amount. In certain embodiments the modified IL-12 polypeptide is administered intratumorally.

The invention also provides a method of treating cancer comprising administering a modified IL-12 polynucleotide of the invention to a patient in an amount sufficient to produce a therapeutically effective dose of modified IL-12 polypeptide. In certain embodiments the modified IL-12 polypeptide is administered intratumorally. In additional embodiments, the modified IL-12 polynucleotide is contained in an expression vector. In a preferred embodiment, the expression vector is an adenoviral vector or adeno-associated viral (AAV) vector.

The modified IL-12 polynucleotides and polypeptides of the invention may be administered in combination with one or more therapeutic agents and/or procedures in the treatment, prevention, amelioration and/or cure of cancers.

In a specific embodiment, modified IL-12 polynucleotides and polypeptides of the invention are administered in combination with one or more chemotherapeutic useful in the treatment of cancers including, but not limited to Alkylating agents; Nitrogen mustards (mechlorethamine, cyclophosphamide, ifosfamide, melphalan, chlorambucil); Nitrosoureas (carmustine (BCNU), lomustine (CCNU), semustine (methyl-CCNU), Ethylenimine/Methyl-melamine, thriethylenemelamine (TEM), triethylene thiophosphoramide (thiotepa), hexamethylmelamine (HMM, altretamine)); Alkyl sulfonates (busulfan); Triazines (dacarbazine (DTIC)); Folic Acid analogs (methotrexate, Trimetrexate, Pemetrexed); Pyrimidine analogs (5-fluorouracil fluorodeoxyuridine, gemcitabine, cytosine arabinoside (AraC, cytarabine), 5-azacytidine, 2,2′-difluorodeoxy-cytidine); Purine analogs (6-mercaptopurine, 6-thioguanine, azathioprine, 2′-deoxycoformycin (pentostatin), erythrohydroxynonyl-adenine (EHNA), fludarabine phosphate, 2-chlorodeoxyadenosine (cladribine, 2-CdA)); Type I Topoisomerase Inhibitors (camptothecin, topotecan, irinotecan); Biological response modifiers (IL-2, G-CSF, GM-CSF); Differentiation Agents (retinoic acid derivatives, Hormones and antagonists); Adrenocorticosteroids/antagonists (prednisone and equivalents, dexamethasone, ainoglutethimide); Progestins (hydroxyprogesterone caproate, medroxyprogesterone acetate, megestrol acetate); Estrogens (diethylstilbestrol, ethynyl estradiol/equivalents); Antiestrogen (tamoxifen); Androgens (testosterone propionate, fluoxymesterone/equivalents); Antiandrogens (flutamide, gonadotropin-releasing hormone analogs, leuprolide); Nonsteroidal antiandrogens (flutamide); Natural products; Antimitotic drugs; Taxanes (paclitaxel, Vinca alkaloids, vinblastine (VLB), vincristine, vinorelbine, Taxotere (docetaxel), estramustine, estramustine phosphate); Epipodophylotoxins (etoposide, teniposide); Antibiotics (actimomycin D, daunomycin (rubido-mycin), doxorubicin (adria-mycin), mitoxantroneidarubicin, bleomycin, splicamycin (mithramycin), mitomycinC, dactinomycin, aphidicolin); Enzymes (L-asparaginase, L-arginase); Radiosensitizers (metronidazole, misonidazole, desmethylmisonidazole, pimonidazole, etanidazole, nimorazole, RSU 1069, EO9, RB 6145, SR4233, nicotinamide, 5-bromodeozyuridine, 5-iododeoxyuridine, bromodeoxycytidine); Platinium coordination complexes (cisplatin, Carboplatin, oxaliplatin, Anthracenedione, mitoxantrone); Substituted urea (hydroxyurea); Oxazaphosphorines (cyclophosphamide; ifosfamide; trofosfamide; mafosfamide (NSC 345842), glufosfamide (D19575, beta-D-glucosylisophosphoramide mustard), S-(−)-bromofosfamide (CBM-11), NSC 612567 (aldophosphamide perhydrothiazine); NSC 613060 (aldophosphamide thiazolidine); isophosphoramide mustard; palifosfamide lysine); Methylhydrazine derivatives (N-methylhydrazine (MIH), procarbazine); Adrenocortical suppressant (mitotane (o,p′-DDD), ainoglutethimide); Cytokines (interferon (alpha, beta, gamma), interleukin-2); Photosensitizers (hematoporphyrin derivatives, Photofrin, benzoporphyrin derivatives, Npe6, tin etioporphyrin (SnET2), pheoboride-a, bacteriochlorophyll-a, naphthalocyanines, phthalocyanines, zinc phthalocyanines); and Radiation (X-ray, ultraviolet light, gamma radiation, visible light, infrared radiation, microwave radiation).

### Modes of Administration

The modified IL-12 polypeptides and polynucleotides may be administered to the subject systemically or locally (e.g., at the site of the disease or disorder). Systemic administration may be by any suitable method, including subcutaneously and intravenously. Local administration may be by any suitable method, including without limitation, intraperitoneally, intrathecally, intraventricularly, or by direct injection into a tissue or organ, such as intratumoral injection.

In certain embodiments, modified IL-12 polynucleotide expression is controlled by a ligand-inducible gene switch system, such as described, for example, in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. application Ser. No. 10/468,193 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); and, U.S. application Ser. No. 12/241,018 (U.S. Pub. No. 20090136465). In these embodiments, once the modified IL-12 polynucleotides under the control of a gene switch have been introduced to the subject, an activating ligand may be administered to induce expression of the modified IL-12 polypeptide of the invention. The ligand may be administered by any suitable method, either systemically (e.g., orally, intravenously) or locally (e.g., intraperitoneally, intrathecally, intraventricularly, direct injection into the tissue or organ where the disease or disorder is occurring, including intratumorally). The optimal timing of ligand administration can be determined for each type of cell and disease or disorder using only routine techniques.

In certain embodiments, modified IL-12 polynucleotides are introduced into in vitro engineered cells such as immune cells (e.g., dendritic cells, T cells, Natural Killer cells) or stem cells (e.g., mesenchymal stem cells, endometrial stem cells, embryonic stem cells), which conditionally express a modified IL-12 polypeptide under the control of a gene switch, which can be activated by an activating ligand. Such methods are described in detail, for example, in: PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. application Ser. No. 14/001,943 (U.S. Pub. No. [Pending]).

In one embodiment, immune cells or stem cells are transfected with an adenovirus vector or an adeno-associated virus vector comprising a modified IL-12 polynucleotide to produce in vitro engineered cells.

In one embodiment the in vitro engineered immune cells or stem cells are autologous cells. In another embodiment the in vitro engineered immune cells or stem cells are allogeneic.

One embodiment of the invention provides a method for treating a tumor, comprising the steps in order of: 1) administering intra-tumorally in a mammal a population of in vitro engineered immune cells or stem cells containing a modified IL-12 vector under the control of a gene switch; and 2) administering to said mammal a therapeutically effective amount of an activating ligand.

In certain embodiments the mammal is a human. In other embodiments the mammal is a dog, a cat, or a horse.

In one embodiment, the activating ligand is administered at substantially the same time as the composition comprising the in vitro engineered cells or the vector, e.g., adenoviral or adeno-associated viral vector, e.g., within one hour before or after administration of the cells or the vector compositions. In another embodiment, the activating ligand is administered at or less than about 24 hours after administration of the in vitro engineered immune cells or stem cells, or the vector. In still another embodiment, the activating ligand is administered at or less than about 48 hours after the in vitro engineered immune cells or stem cells, or the vector. In another embodiment, the ligand is RG-115932. In another embodiment, the ligand is administered at a dose of about 1 to 50 mg/kg/day. In another embodiment, the ligand is administered at a dose of about 30 mg/kg/day. In another embodiment, the ligand is administered daily for a period of 7 to 28 days. In another embodiment, the ligand is administered daily for a period of 14 days. In another embodiment, about 1×10⁶ to 1×10⁸ cells are administered. In another embodiment, about 1×10⁷ cells are administered.

Having provided for the substantially pure polypeptides, biologically active fragments thereof and recombinant polynucleotides encoding them, the present invention also provides cells comprising each of them. By appropriate introduction techniques well known in the field, cells comprising them may be produced. See, e.g., Sambrook et al. (1989).

### Host Cells and Non-Human Organisms

Another aspect of the present invention involves cells comprising an isolated polynucleotide encoding a modified IL-12 polypeptide of the present invention. In a specific embodiment, the invention relates to an isolated host cell comprising a vector comprising a polynucleotide encoding a modified IL-12 polypeptide of the present invention. The present invention also relates to an isolated host cell comprising an expression vector according to the invention. In another specific embodiment, the invention relates to an isolated host cell comprising a gene expression cassette comprising a polynucleotide encoding a modified IL-12 polypeptide of the present invention. In another specific embodiment, the invention relates to an isolated host cell transfected with a gene expression modulation system comprising a polynucleotide encoding a modified IL-12 polypeptide of the present invention. In still another embodiment, the invention relates to a method for producing a modified IL-12 polypeptide, wherein the method comprises culturing an isolated host cell comprising a polynucleotide encoding a modified IL-12 polypeptide of the present invention in culture medium under conditions permitting expression of the polynucleotide encoding the modified IL-12 polypeptide, and isolating the modified IL-12 polypeptide from the culture.

In one embodiment, the isolated host cell is a prokaryotic host cell or a eukaryotic host cell. In another specific embodiment, the isolated host cell is an invertebrate host cell or a vertebrate host cell. Preferably, the isolated host cell is selected from the group consisting of a bacterial cell, a fungal cell, a yeast cell, a nematode cell, an insect cell, a fish cell, a plant cell, an avian cell, an animal cell, and a mammalian cell. For example but without limitation, the isolated host cell may be a yeast cell, a nematode cell, an insect cell, a plant cell, a zebrafish cell, a chicken cell, a hamster cell, a mouse cell, a rat cell, a rabbit cell, a cat cell, a dog cell, a bovine cell, a goat cell, a cow cell, a pig cell, a horse cell, a sheep cell, or a non-human primate cell (for example, a simian cell, a monkey cell, a chimpanzee cell), or a human cell.

Examples of host cells include, but are not limited to, fungal or yeast species such as Aspergillus, Trichoderma, Saccharomyces, Pichia, Candida, Hansenula, or bacterial species such as those in the genera Synechocystis, Synechococcus, Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella; animal; and mammalian host cells.

In one embodiment, the isolated host cell is a yeast cell selected from the group consisting of a Saccharomyces, a Pichia, and a Candida host cell.

In another embodiment, the isolated host cell is a Caenorhabdus elegans nematode cell.

In another embodiment, the isolated host cell is a mammalian cell selected from the group consisting of a hamster cell, a mouse cell, a rat cell, a rabbit cell, a cat cell, a dog cell, a bovine cell, a goat cell, a cow cell, a pig cell, a horse cell, a sheep cell, a non-human primate cell (such as a monkey cell or a chimpanzee cell), and a human cell.

Host cell transformation is well known in the art and may be achieved by a variety of methods including but not limited to electroporation, viral infection, plasmid/vector transfection, non-viral vector mediated transfection, Agrobacterium-mediated transformation, particle bombardment, and the like. Expression of desired gene products involves culturing the transformed host cells under suitable conditions and inducing expression of the transformed gene. Culture conditions and gene expression protocols in prokaryotic and eukaryotic cells are well known in the art (see General Methods section of Examples). Cells may be harvested and the gene products isolated according to protocols specific for the gene product.

In addition, a host cell may be chosen that modulates the expression of the transfected polynucleotide, or modifies and processes the polypeptide product in a specific fashion desired. Different host cells have characteristic and specific mechanisms for the translational and post-translational processing and modification [e.g., glycosylation, cleavage (e.g., of signal sequence)] of proteins. Appropriate cell lines or host systems can be chosen to ensure the desired modification and processing of the foreign protein expressed. For example, expression in a bacterial system can be used to produce a non-glycosylated core protein product. However, a polypeptide expressed in bacteria may not be properly folded. Expression in yeast can produce a glycosylated product. Expression in eukaryotic cells can increase the likelihood of “native” glycosylation and folding of a heterologous protein. Moreover, expression in mammalian cells can provide a tool for reconstituting, or constituting, the polypeptide's activity. Furthermore, different vector/host expression systems may affect processing reactions, such as proteolytic cleavages, to a different extent.

Applicants' invention also relates to a non-human organism comprising an isolated host cell according to the invention. In a specific embodiment, the non-human organism is a prokaryotic organism or a eukaryotic organism. In another specific embodiment, the non-human organism is an invertebrate organism or a vertebrate organism.

In certain embodiments, the non-human organism is selected from the group consisting of a bacterium, a fungus, a yeast, a nematode, an insect, a fish, a plant, a bird, an animal, and a mammal. More preferably, the non-human organism is a yeast, a nematode, an insect, a plant, a zebrafish, a chicken, a hamster, a mouse, a rat, a rabbit, a cat, a dog, a bovine, a goat, a cow, a pig, a horse, a sheep, or a non-human primate (such as a simian, a monkey, or a chimpanzee).

The present invention may be better understood by reference to the following non-limiting Examples, which are provided as exemplary of the invention.

### EXAMPLES

### General Molecular Biology Techniques

In accordance with the present invention there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, N.Y. (herein “Green & Sambrook, 2012”); DNA Cloning: A Practical Approach, Volumes I and II, Second Edition (D. M. Glover and B. D. Hames, eds. 1995); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Culture of Animal Cells: A Manual of Basic Technique and Specialized Applications [R. I. Freshney (2010)]; Immobilized Cells And Enzymes [IRL Press, (1986)]; B. Perbal, A Practical Guide To Molecular Cloning, Second Edition (1988); F. M. Ausubel et al. (eds.), Current Protocols in Molecular Biology, John Wiley & Sons, Inc. (2013).

Conventional cloning vehicles include pBR322 and pUC type plasmids and phages of the M13 series. These may be obtained commercially (e.g., Life Technologies Corporation; Promega Corporation).

For ligation, DNA fragments may be separated according to their size by agarose or acrylamide gel electrophoresis, extracted with phenol or with a phenol/chloroform mixture, precipitated with ethanol and then incubated in the presence of phage T4 DNA ligase (New England Biolabs, Inc.) according to the supplier's recommendations.

The filling in of 5′ protruding ends may be performed with the Klenow fragment of E. coli DNA polymerase I (New England Biolabs, Inc.) according to the supplier's specifications. The destruction of 3′ protruding ends is performed in the presence of phage T4 DNA polymerase (New England Biolabs, Inc.) used according to the manufacturer's recommendations. The destruction of 5′ protruding ends is performed by a controlled treatment with S1 nuclease.

Mutagenesis directed in vitro by synthetic oligodeoxynucleotides may be performed according to the method developed by Taylor et al. [Nucleic Acids Res. 13 (1985) 8749-8764] using commercial kits such as those distributed by Life Technologies Corp. and Agilent Technologies, Inc.

The enzymatic amplification of DNA fragments by PCR [Polymerase-catalyzed Chain Reaction, Saiki R. K. et al., Science 230 (1985) 1350-1354; Mullis K. B. and Faloona F. A., Meth. Enzym. 155 (1987) 335-350] technique may be performed using a “DNA thermal cycler” (Life Technologies Corp.) according to the manufacturer's specifications.

Verification of nucleotide sequences may be performed by the method developed by Sanger et al. [Proc. Natl. Acad. Sci. USA, 74 (1977) 5463-5467] using commercial kits such as those distributed by GE Healthcare and Life Technologies Corp.

Plasmid DNAs may be purified by the Qiagen Plasmid Purification System according to the manufacture's instruction.

Embodiments (E) of the Invention Comprise (without Limitation):

E1. A modified single-chain IL-12 polypeptide comprising, from N- to C-terminus:

i. a first IL-12 p40 domain (p40N),

ii. an optional first peptide linker,

iii. an IL-12 p35 domain,

iv. an optional second peptide linker, and

v. a second IL-12 p40 domain (p40C);
wherein the first IL-12 p40 domain (p40N) is an N-terminal fragment of a p40 subunit; the IL-12 p35 domain is a mature p35 subunit or fragment thereof; and the second IL-12 p40 domain (p40C) is a C-terminal fragment of a p40 subunit;
   except wherein one or more portions of the polypeptide are engineered to comprise naturally occurring or synthetically (artificially) derived proteolytic target sites.

E2. The single chain IL-12 polypeptide of E1, which comprises an N-terminal signal peptide domain.

E3. The single chain IL-12 polypeptide of E1 comprising amino acids 23 to 533 of SEQ ID NO: 10.

E4. The single chain IL-12 polypeptide of E1 comprising the amino acid sequence of SEQ ID NO: 10.

E5. The single chain IL-12 polypeptide of E1 wherein the first and second peptide linkers are selected from Thr-Pro-Ser (SEQ ID NO: 41) and Ser-Gly-Pro-Ala-Pro (SEQ ID NO: 42).

E6. The single chain IL-12 polypeptide of E1 which lacks a first peptide linker.

E7. The single chain IL-12 polypeptide of E1 which lacks a second peptide linker.

E8. A polynucleotide comprising a nucleic acid sequence encoding the single chain IL-12 polypeptide of E1.

E9. The polynucleotide of E8 which comprises nucleic acids 67 to 1599 of SEQ ID NO: 9.

E10. A vector comprising the polynucleotide of E8.

E11. The vector of E10 which is an adenovirus or adeno-associated virus vector.

E12. An isolated host cell or a non-human organism transformed or transfected with the vector of E10.

E13. The isolated host cell of E12 which is an immune cell or a stem cell.

E14. A method of enhancing the immune response of a patient comprising administering an effective amount of the single chain IL-12 polypeptide of E1.

E15. A method of enhancing the immune response of a patient comprising administering an effective amount of the polynucleotide of E8.

E16. A method of enhancing the immune response of a patient comprising administering an effective amount of the vector of E10.

E17. A method of enhancing the immune response of a patient comprising administering an effective amount of the host cell of E12.

### Further Embodiments (FE) of the Invention Comprise (without Limitation)

FE1. An interleukin-12 (IL-12) composition wherein said composition has been modified to have a reduced half-life compared to a corresponding non-modified IL-12 composition.

FE2. The composition of FE1, wherein said IL-12 composition comprises one or more amino acid substitutions which increase the rate of proteolysis of said composition compared to the rate of proteolysis of a corresponding IL-12 composition not having said one or more amino acid substitutions.

FE3. The composition of FE2, wherein said IL-12 composition is a heterodimer of p40 and p35 polypeptides.

FE4. The composition of FE2, wherein the corresponding non-modified IL-12 composition is a heterodimer of human IL-12 p40 and human IL-12 p35 polypeptides.

FE5. The composition of FE2, wherein said IL-12 composition is a single chain IL-12 polypeptide.

FE6. The composition of FE2, wherein said IL-12 composition is a topologically manipulated single chain IL-12 polypeptide.

FE7. The composition of FE2, wherein said IL-12 composition comprises a p40 polypeptide which comprises any one or more amino acid substitutions selected from the group consisting of:

K126L
K124G/K126L
K124A/K126L
K124S/K126L
K124G/N125G/K126L
K124A/N125A/K126L
M45L
N248L
K247A/N248L
L246A/K247A/N248L
L246S/K247A/N248L
A172P
A172P/T174A
D40A/P42L
G161P/D164L
K126L
K124G/K126L
K124A/K126L
K124S/K126L
K124G/N125G/K126L
K124A/N125A/K126L
M45L
D287S
K302S/N303S
V180S
K280L/S281V/K282P/E284G/K285S
S176L/A177V/E178P/V180T/R181S
K280L/S281V/K282P/E284G/K285V
S176L/A177V/E178P/V180S/R181S
N248S/S249G
K282G/K285V
S249G
K282G/K307V

wherein these substitution positions correspond to amino acid positions as shown in SEQ ID NO:2.

FE8. The composition of FE2, wherein said IL-12 composition comprises a p35 polypeptide which comprises any one or more amino acid substitutions selected from the group consisting of:

Q186L
S215L
Y223L
K214P
K214P/S216A
C144P/S147L
C144P/L145S/S147L
G142R/R148G
K149S
K149A
E135S
Q186S
S216R
D111A/K112R
Q213R/K214L/S215R/S216A
A146V/S147P/K149G/T150S/S151K
N132V/S133P/E135G/T136S/S137K
S147P/K149I/T150I/S151K
N132F/S133P/E135G/S137K
N77I/L78P/S83R
T210L/Q213R/K214G
R148G/K149R
N207S/S208G/E209R
E209G/T210R

wherein these substitution positions correspond to amino acid positions as shown in SEQ ID NO:4.

FE9. The composition of FE2, wherein said IL-12 composition comprises a topologically manipulated single chain IL-12 polypeptide which comprises any one or more amino acid substitutions selected from the group consisting of:

K126L
K124G/K126L
K124A/K126L
K124S/K126L
K124G/N125G/K126L
K124A/N125A/K126L
M45L
N248L
K247A/N248L
L246A/K247A/N248L
L246S/K247A/N248L
Q426L
S455L
Y463L
A172P
A172P/T174A
K454P
K454P/S456A
C384P/S387L
C384P/L385S/S387L
D40A/P42L
G161P/D164L
D287S
K302S/N303S
V180S
G382R/R388G
K389S
K389A
E375S
Q426S
S456R
D351A/K352R
Q453R/K454L/S455R/S456A
K280L/S281V/K282P/E284G/K285S
S176L/A177V/E178P/V180T/R181S
A386V/S387P/K389G/T390S/S391K
N372V/S373P/E375G/T377S/S378K
K280L/S281V/K282P/E284G/K285V
S176L/A177V/E178P/V180S/R181S
S365P/K367I/T368I/S369K
N372F/S373P/E375G/S377K
N317I/L319P/S323R
T450L/Q453R/K454G
K280L/S281V/K282P/E284G/K285S
N248S/S249G
K282G/K285V
S249G
K282G/K285V
R388G/K389R
N447S/S448G/E449R
E449G/T450R

wherein these substitution positions correspond to amino acid positions as shown in SEQ ID NO:10.

FE10. An interleukin-12 (IL-12) composition wherein said composition has been modified to comprise a membrane linking (tethering/anchoring/binding) moiety.

FE11. The composition of FE10, wherein said IL-12 composition comprises one or more amino acid substitutions which increase the rate of proteolysis of said composition compared to the rate of proteolysis of a corresponding IL-12 composition not having said one or more amino acid substitutions.

FE12. The composition of FE10, wherein said IL-12 composition comprises a heterodimer of p40 and p35 polypeptides.

FE13. The composition of FE11, wherein the corresponding non-modified IL-12 composition is a heterodimer of human IL-12 p40 and human IL-12 p35 polypeptides.

FE14. The composition of FE10, wherein said IL-12 composition comprises a single chain IL-12 polypeptide.

FE15. The composition of FE10, wherein said IL-12 composition comprises a topologically manipulated single chain IL-12 polypeptide.

FE16. The composition of any one of FE10 to FE15, wherein said membrane anchoring, linking, or tethering) moiety is selected from the group consisting of: a covalent membrane surface linking moiety, a hydrophobic membrane surface linking moiety, a hydrophillic membrane surface linking moiety, an ionic membrane surface linking moiety, an integral membrane polypeptide, and a transmembrane polypeptide.

FE17. The composition in any one of FE1 to FE16, wherein IL-12 expression is inducibly regulated by a gene switch.

FE18. The composition of FE17, wherein said gene switch is an ecdysone receptor-based (EcR-based) switch

FE19. The composition in any one of FE1 to FE18, wherein said IL-12 is expressed by a modified T cell.

FE20. The composition of FE19, wherein said modified T cell is a modified autologous T cell.

FE21. A method of treating a cancer or immune system disorder comprising administering a therapeutically useful amount of the composition in any one of FE1 to FE20.

### Additional Embodiments (AE) of the Invention Comprise (without Limitation)

AE1. An interleukin-12 (IL-12) composition wherein said composition has been modified to have a reduced half-life compared to a corresponding non-modified IL-12 composition.

AE2. The composition of AE1, wherein said IL-12 composition comprises one or more amino acid substitutions which increase the rate of proteolysis of said composition compared to the rate of proteolysis of a corresponding IL-12 composition not having said one or more amino acid substitutions.

AE3. The composition of AE2, wherein said IL-12 composition is a heterodimer of p40 and p35 polypeptides.

AE4. The composition of AE2, wherein the corresponding non-modified IL-12 composition is a heterodimer of human IL-12 p40 and human IL-12 p35 polypeptides.

AE5. The composition of AE2, wherein said IL-12 composition is a single chain IL-12 polypeptide.

AE6. The composition of AE2, wherein said IL-12 composition is a topologically manipulated single chain IL-12 polypeptide.

AE7. The composition of AE2, wherein said IL-12 composition comprises a p40 polypeptide which comprises any one or more amino acid substitutions selected from the group consisting of:

K126L
K124G/K126L
K124A/K126L
K124S/K126L
K124G/N125G/K126L
K124A/N125A/K126L
M45L
N248L
K247A/N248L
L246A/K247A/N248L
L246S/K247A/N248L
A172P
A172P/T174A
D40A/P42L
G161P/D164L
K126L
K124G/K126L
K124A/K126L
K124S/K126L
K124G/N125G/K126L
K124A/N125A/K126L
M45L
D287S
K302S/N303S
V180S
K280L/S281V/K282P/E284G/K285S
S176L/A177V/E178P/V180T/R181S
K280L/S281V/K282P/E284G/K285V
S176L/A177V/E178P/V180S/R181S
N248S/S249G
K282G/K285V
S249G
K282G/K307V

wherein these substitution positions correspond to amino acid positions as shown in SEQ ID NO: 2.

AE8. The composition of AE2, wherein said IL-12 composition comprises a p35 polypeptide which comprises any one or more amino acid substitutions selected from the group consisting of:

Q186L
S215L
Y223L
K214P
K214P/S216A
C144P/S147L
C144P/L145S/S147L
G142R/R148G
K149S
K149A
E135S
Q186S
S216R
D111A/K112R
Q213R/K214L/S215R/S216A
A146V/S147P/K149G/T150S/S151K
N132V/S133P/E135G/T136S/S137K
S147P/K149I/T150I/S151K
N132F/S133P/E135G/S137K
N77I/L78P/S83R
T210L/Q213R/K214G
R148G/K149R
N207S/S208G/E209R
E209G/T210R

wherein these substitution positions correspond to amino acid positions as shown in SEQ ID NO: 4.

AE9. The composition of AE2, wherein said IL-12 composition comprises a topologically manipulated single chain IL-12 polypeptide which comprises any one or more amino acid substitutions selected from the group consisting of:

K126L
K124G/K126L
K124A/K126L
K124S/K126L
K124G/N125G/K126L
K124A/N125A/K126L
M45L
N248L
K247A/N248L
L246A/K247A/N248L
L246S/K247A/N248L
Q426L
S455L
Y463L
A172P
A172P/T174A
K454P
K454P/S456A
C384P/S387L
C384P/L385S/S387L
D40A/P42L
G161P/D164L
D287S
K302S/N303S
V180S
G382R/R388G
K389S
K389A
E375S
Q426S
S456R
D351A/K352R
Q453R/K454L/S455R/S456A
K280L/S281V/K282P/E284G/K285S
S176L/A177V/E178P/V180T/R181S
A386V/S387P/K389G/T390S/S391K
N372V/S373P/E375G/T377S/S378K
K280L/S281V/K282P/E284G/K285V
S176L/A177V/E178P/V180S/R181S
S365P/K367I/T368I/S369K
N372F/S373P/E375G/S377K
N317I/L319P/S323R
T450L/Q453R/K454G
K280L/S281V/K282P/E284G/K285S
N248S/S249G
K282G/K285V
S249G
K282G/K285V
R388G/K389R
N447S/S448G/E449R
E449G/T450R

wherein these substitution positions correspond to amino acid positions as shown in SEQ ID NO:10.

AE10. An interleukin-12 (IL-12) composition wherein said composition has been modified to comprise a membrane linking (tethering/anchoring/binding) moiety.

AE11. The composition of AE10, wherein said IL-12 composition comprises one or more amino acid substitutions which increase the rate of proteolysis of said composition compared to the rate of proteolysis of a corresponding IL-12 composition not having said one or more amino acid substitutions.

AE12. The composition of AE10, wherein said IL-12 composition comprises a heterodimer of p40 and p35 polypeptides.

AE13. The composition of AE11, wherein the corresponding non-modified IL-12 composition is a heterodimer of human IL-12 p40 and human IL-12 p35 polypeptides.

AE14. The composition of AE10, wherein said IL-12 composition comprises a single chain IL-12 polypeptide.

AE15. The composition of AE10, wherein said IL-12 composition comprises a topologically manipulated single chain IL-12 polypeptide.

AE16. The composition of any one of AE10 to AE15, wherein said membrane anchoring, linking, or tethering) moiety is selected from the group consisting of: a covalent membrane surface linking moiety, a hydrophobic membrane surface linking moiety, a hydrophillic membrane surface linking moiety, an ionic membrane surface linking moiety, an integral membrane polypeptide, and a transmembrane polypeptide.

AE17. The composition in any one of AE10 to AE15, wherein IL-12 expression is inducibly regulated by a gene switch.

AE18. The composition of AE16, wherein IL-12 expression is inducibly regulated by a gene switch.

AE19. The composition of AE17, wherein said gene switch is an ecdysone receptor-based (EcR-based) switch.

AE20. The composition of AE18, wherein said gene switch is an ecdysone receptor-based (EcR-based) switch.

AE21. The composition of AE19, wherein said gene switch is an ecdysone receptor-based (EcR-based) switch.

AE22. The composition of AE20, wherein said IL-12 is expressed by a modified T cell.

AE23. The composition of AE21, wherein said IL-12 is expressed by a modified T cell.

AE24. A method of treating a cancer or an immune system disorder comprising administering a therapeutically useful amount of the composition of AE17.

AE25. A method of treating a cancer or immune system disorder comprising administering a therapeutically useful amount of the composition of AE18.

### Example 1: Design of scIL-12 Fusion Proteins

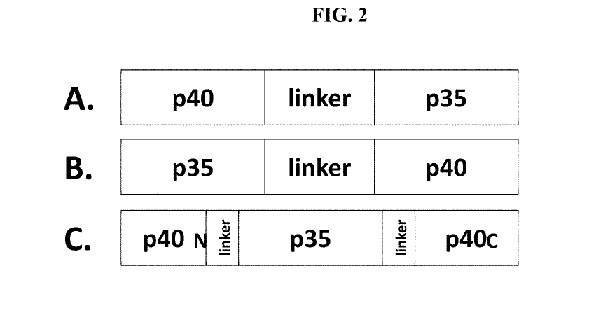

Single chain IL-12 molecules are designed to have one of three configurations, illustrated in FIG. 2:

The p40-linker-p35 configuration (FIG. 2A) contains the full-length p40 subunit (including wild type signal peptide) fused to the mature p35 subunit (without signal peptide) via a peptide linker;

The p35-linker-p40 configuration (FIG. 2B) contains the full-length p35 subunit (including wild type signal peptide) fused to the mature p40 subunit (without signal peptide) via a peptide linker; and

The p40N-p35-p40C insert configuration (FIG. 2C) comprising, from N- to C-terminus:

(i) a first IL-12 p40 domain (p40N),

(ii) an optional first peptide linker,

(iii) an IL-12 p35 domain,

(iv) an optional second peptide linker, and

(v) a second IL-12 p40 domain (p40C).

Specific human scIL-12 constructs are summarized in Table 1. Amino acid residues specified by number in the Description column refer to the amino acid numbering of the full-length human p40 or p35 subunits shown in SEQ ID NOs: 2 and 4, respectively. For example, the nucleic acid and amino acid sequences of scIL-12 Construct ID 1481273, corresponding to SEQ ID NOs: 9 and 10, respectively, is a p40N-p35-p40C insert configuration; and was designed to contain, from N- to C-terminus, a first p40 domain (p40N) consisting of amino acids 1 to 293 of SEQ ID NO: 2, a first linker sequence of TPS (Thr-Pro-Ser; SEQ ID NO: 41), a mature p35 sequence consisting of amino acids 57 to 253 of SEQ ID NO: 4, a second peptide linker sequence of GPAPTS (Gly-Pro-Ala-Pro-Thr-Ser; SEQ ID NO: 82), and a second p40 domain (p40C) consisting of amino acids 294 to 328 of SEQ ID NO: 2.

Construct ID 1481272 (SEQ ID NOs: 11 and 12) is also a p40N-p35-40C insert configuration, but the p35 insert occurs between amino acid residues 259 and 260 of the p40 subunit.

The remaining scIL-12 designs (Construct IDs 1480533 to 1480546) represent p40-p35 or p35-p40 single chain IL-12 molecules with various linkers as indicated in Table 1.

Parallel mouse constructs were also designed, using the mouse p40 and p35 sequences (SEQ ID NOs: 5-8) instead of human IL-12 sequences.

TABLE 14
Human scIL-12 constructs

| | DNA | Protein | |
|---|---|---|---|
| Construct | SEQ | SEQ | |
| ID | ID NO | ID NO | Description |
| 1481273 | 9 | 10 | p40N₍₁₋₂₉₃₎-TPS-p35₍₅₇₋₂₅₃₎-GPAPTS-p40C₍₂₉₄₋₃₂₈₎ |
| 1481272 | 11 | 12 | p40N₍₁₋₂₅₉₎-GS-p35₍₅₇₋₂₅₃₎-PQTPGP-p40C₍₂₆₀₋₃₂₈₎ |
| 1480533 | 13 | 14 | p40₍₁₋₃₂₈₎-RSPVSGDNAFPAPTG-p35₍₅₇₋₂₅₃₎ |
| 1480534 | 15 | 16 | p40₍₁₋₃₂₈₎-RSQPVPTRDLEVPLTG-p35₍₅₇₋₂₅₃₎ |
| 1480535 | 17 | 18 | p40₍₁₋₃₂₈₎-RSGTPPQTGLEKPTGTG-p35₍₅₇₋₂₅₃₎ |
| 1480536 | 19 | 20 | p40₍₁₋₃₂₈₎-SDVTGNTGNATYTIT-p35₍₅₇₋₂₅₃₎ |
| 1480537 | 21 | 22 | p40₍₁₋₃₂₈₎-GSPKDGPEIPPTGGT-p35₍₅₇₋₂₅₃₎ |
| 1480538 | 23 | 24 | p40₍₁₋₃₂₈₎-GRNAPGSPPTGNYKLEP-p35₍₅₇₋₂₅₃₎ |
| 1480539 | 25 | 26 | p40₍₁₋₃₂₈₎-QKGSVGFTDPEVHQSTNL-p35₍₅₇₋₂₅₃₎ |
| 1480540 | 27 | 28 | p40₍₁₋₃₂₈₎-GNVPELPDTTEHSRT-p35₍₅₇₋₂₅₃₎ |
| 1480541 | 29 | 30 | p40₍₁₋₃₂₈₎-GRSHPVQPYPGAFVKEPIP-p35₍₅₇₋₂₅₃₎ |
| 1480542 | 31 | 32 | p40₍₁₋₃₂₈₎-PERKERISEQTYQLS-p35₍₅₇₋₂₅₃₎ |
| 1480543 | 33 | 34 | p40₍₁₋₃₂₈₎-(G₄S)₃-p35₍₅₇₋₂₅₃₎ |
| 1480544 | 35 | 36 | p40₍₁₋₃₂₈₎-G₆S-p35₍₅₇₋₂₅₃₎ |
| 1480545 | 37 | 38 | p35₍₃₅₋₂₅₃₎-RSDVNSRTGPSGATPPSGNPYTITG-p40₍₂₃₋₃₂₈₎ |
| 1480546 | 39 | 40 | p35₍₃₅₋₂₅₃₎-PAPTPSNGSPKDGPEIPPTGG-p40₍₂₃₋₃₂₈₎ |

Embodiments of the invention include, without limitation, the scIL-12 constructs indicated in Table 14 above. The scIL-12 constructs of the invention may comprise, or may not comprise, a signal peptide sequence (whether synthesized with or without a signal peptide or as may occur as a result of polypeptide cleavage in the secreted form subsequent to in vitro or in vivo expression and post-translational processing). For example, but without limitation, with respect to scIL-12 Construct No. 1481273 (p40N₍₁₋₂₉₃₎-TPS-p35₍₅₇₋₂₅₃₎-GPAPTS-p40C₍₂₉₄₋₃₂₈₎ (SEQ ID NO: 10)) embodiments of the invention also include this polypeptide sequence without a signal peptide (e.g., p40N₍₂₃₋₂₉₃₎-TPS-p35₍₅₇₋₂₅₃₎-GPAPTS-p40C₍₂₉₄₋₃₂₈₎ (residues 23-533 of SEQ ID NO: 10)). Likewise, without limitation, embodiments of the invention include any of the remaining scIL-12 constructs shown in Table 14 without a signal peptide.

In some embodiments, other protease target sequences can be used, e.g., human protease target sequences. Examples of representative naturally-occurring protease target sequences include, but are not limited, to those found in Table X.

TABLE X*

| | | SEQ | |
|---|---|---|---|
| | | ID | |
| Protein | Target Sequence | NO: | Protease |
| AGT | YIHPFHLV | 84 | angiotensin converting enzyme |
| AGT | HPFHLVIH | 85 | angiotensin converting enzyme |
| CDCP1 | KQSRKFVP | 86 | urokinase plasminogen activator/matriptase |
| CP | PQSRSVPP | 87 | plasmin |
| CP | RRPYLKVFNPRRKLEF | 88 | plasmin |
| CPB2 | VSPRASAS | 89 | thrombin |
| F12 | SMTRVVGG | 90 | hepsin |
| F2R | LDPRSFLL | 91 | thrombin |
| F2RL1 | SKGRSLIG | 92 | plasmin |
| F5 | LGIRSFRN | 93 | thrombin |
| F9 | HKGRSALV | 94 | thrombin |
| GFB | FSARGHRP | 83 | thrombin |
| GHRL | KLQPRALA | 95 | angiotensin converting enzyme |
| GRIN1 | NALRYAPD | 96 | tissue plasminogen activator |
| GRIN2A | PEAKASCYG | 97 | plasmin |
| HGF | KTKQLRVVN | 63 | urokinase plasminogen activator/matriptase |
| HGFAC | LRPRIIGG | 98 | thrombin |
| IGFBP | QRYKVDYE | 99 | plasmin |
| IGFBP3 | SRLRAYLL | 100 | thrombin |
| IGFBP5 | AHPRIISA | 101 | thrombin |
| ITGA6 | PSSRRRVNSL | 102 | urokinase plasminogen activator |
| KLKB1 | SKLRVVGG | 103 | plasmin |
| MAPT | VEVKSEKL | 104 | thrombin |
| MMP1 | VEKRRNSGP | 105 | plasma kallikrein |
| MMP13 | KKPRCGVP | 106 | plasmin |
| MMP14 | ANVRRKRYAIQG | 107 | plasmin |
| MST1 | SKLRVVGG | 103 | plasma kallikrein |
| PI3 | QEPVKGPV | 108 | neutrophil elastase |
| PLAT | PQFRIKGG | 62 | plasmin |
| PLAUR | NSGRAVTYSRSRYL | 109 | plasmin |
| PLG | CPGRVVGG | 110 | tissue plasminogen activator |
| SERPINA1 | FLEAIPMSIPPEV | 111 | neutrophil elastase |
| SERPINA10 | LSEITAYSMPPVI | 112 | factor XI |
| SERPINA2 | HLEEKAWSKYQTV | 113 | |
| SERPINA3 | AVKITLLSALVET | 114 | cathepsin G |
| SERPINA4 | TFAIKFFSAQTNR | 115 | plasma kallikrein |
| SERPINA5 | GTIFTFRSARLNS | 116 | protein C |
| SERPINA9 | TTKFIVRSKDGPS | 117 | |
| SERPINB1 | AGIATFCMLMPEE | 118 | neutrophil elastase |
| SERPINB10 | GSEIDIRIRVPSI | 119 | thrombin |
| SERPINB11 | GDSIAVKSLPMRA | 120 | |
| SERPINB12 | GAVVSERSLRSWV | 121 | trypsin |
| SERPINB13 | GIGFTVTSAPGHE | 122 | cathepsin L |
| SERPINB2 | GGVMTGRTGHGGP | 123 | urokinase plasminogen activator |
| SERPINB3 | AVVGFGSSPTSTN | 124 | cathepsin L |
| SERPINB4 | AVVVVELSSPSTN | 125 | cathepsin G |
| SERPINB6 | AAIMMMRCARFVP | 126 | cathepsin G |
| SERPINB7 | GSNIVEKQLPOST | 127 | |
| SERPINB8 | AVVRNSRCSRMEP | 128 | furin |
| SERPINB9 | SCFVVAECCMESG | 129 | granzyme B |
| SERPINC1 | AVVIAGRSLNPNR | 130 | thrombin |
| SERPIND1 | TVGFMPLSTQVRF | 131 | thrombin |
| SERPINE1 | AVIVSARMAPEEI | 132 | tissue plasminogen activator |
| SERPINE2 | TAILIARSSPPWF | 133 | tissue plasminogen activator |
| SERPINF2 | SIAMSRMSLSSFS | 134 | plasmin |
| SERPING1 | AISVARTLLVFEV | 135 | C1 esterase |
| SERPINI1 | GMIAISRMAVLYP | 136 | tissue plasminogen activator |
| SERPINI2 | GIHIPVIMSLAQS | 137 | |
| THPO | ASARTTGS | 138 | thrombin |

*Song et al., ″Predicting Serpin/Protease Interactions″. Methods in Enzymology 2011; 501, 237; Rawlings ND, Waller M, Barrett AJ, Bateman A., ″MEROPS: the database of proteolytic enzymes, their substrates and inhibitors″. Nucleic Acids Res. 2014 Jan; 42(Database issue): D503-9. doi: 10.1093/nar/gkt953; Epub 2013 Oct 23. PubMed PMID: 24157837; PubMed Central PMCID: PMC3964991; Igarashi Y., Eroshkin A., Gramatikova S., Gramatikoff G., Zhang Y., Smith J.W., Osterman A.L., Godzik A. ″CutDB: a proteolytic event database″ Nucleic Acids Res. 2007.Example 2: Expression of scIL-12 fusion proteins in CHO cells

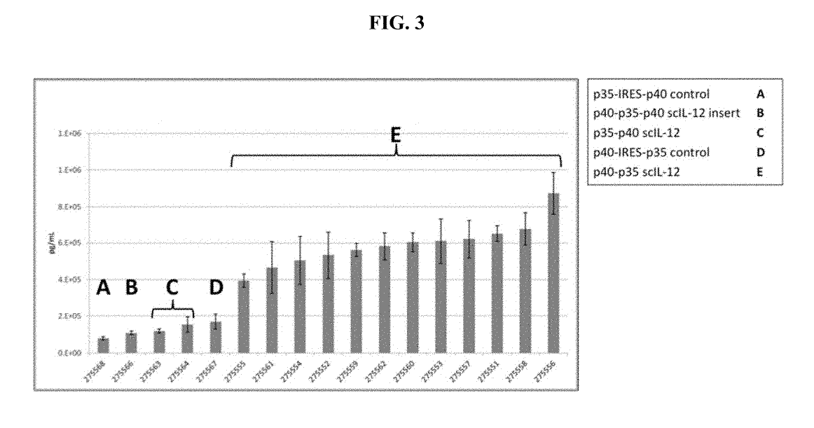

Vectors were constructed containing either human or murine scIL-12 (in all cases cloned between NheI and ClaI sites) along with a 5′UTR element derived from human GAPDH, a synthetic 3′UTR element and with transgene expression under control of a constitutive CMV promoter. Vectors encoding human or mouse scIL-12 constructs were transiently transfected into CHO-K1 cells (ATCC Accession CCL-61) in triplicate using standard high-throughput transfection methods. Briefly, CHO-K1 cells were trypsinized, counted and re-suspended at 120,000 cells/ml in whole growth media (F12-Ham (Sigma)+L-Glutamine (Gibco)+10% FBS (Atlanta Biologicals). One-hundred fifty (150) micro liters of the cell suspension was added to a 96-well cell culture plate (Corning). Plasmid DNA was prepared at 100 ng/μl in sterile water and complexed with Fugene 6 reagent (Promega) at a 3:1 DNA to Fugene 6 ratio. Five (5) micro liters of the DNA/Fugene6 complex was added to the 96-well plate containing the cells. The cells were then incubated at 37° C. for 48 hours. Following incubation the culture supernatant was harvested, and frozen at −80° C. until used for ELISA assays. Positive controls included vectors expressing two-chain IL-12 (p35-IRES-p40 and p40-IRES-p35, labeled in FIG. 3 as bars A and D, respectively). Culture supernatants from transfected CHO-K1 cells were diluted 1:10, 1:100, and 1:1000 in R&D Systems Reagent Diluent+10% conditioned CHO-K1 media.

Expression of scIL-12 was detected by ELISA assays run according to the manufacturer's instructions. R&D Systems, catalog #DY419 (mouse IL-12 ELISA) and #DY1270 (human IL-12 ELISA). Nine samples per vector were analyzed.

Human scIL-12 expression was detected in 20 of the 36 vectors evaluated, and ranged from 500 pg/mL to 900 ng/mL. See FIG. 3. Mouse scIL-12 expression was detected in 18 of the 36 vectors tested. Mouse scIL-12 expression ranged from 385 pg/mL to 1.8 μg/mL (data not shown). For both human and mouse constructs, the p40-linker-p35 configuration demonstrated higher expression levels than the p35-linker-p40 configuration and two-chain (bicistronic) IL-12, suggesting that scIL-12 with p40-linker-p35 topology has enhanced expression, folding and/or heterodimeric assembly as compared to the p35-linker-p40 single chain configuration and two-chain IL-12.

Surprisingly, the human scIL-12 construct ID 1481273, having the configuration:

p40N_{(1 to 293)}-TPS-p35₍₅₇₋₂₅₃₎-GPAPTS-p40C_{(294 to} ₃₂₈₎
(SEQ ID NOS 9 (DNA) and 10 (protein))

resulted in scIL-12 protein expression that was similar to levels produced by two-chain (bicistronic) vectors (p40-IRES-p35 and p35-IRES-p40) and single chain p35-linker-p40 configuration, although not as high as the p40-linker-p35 configuration. See FIG. 3. Similar expression patterns were observed for the mouse scIL-12 designs. Construct ID 1481272, having the configuration p40N₍₁₋₂₅₉₎-GS-p35₍₅₇₋₂₅₃₎-PQTPGP-p40C₍₂₆₀₋₃₂₈₎ (SEQ ID NOS 11 (DNA) and 12 (protein)), was found not to express detectable protein.

### Example 3: scIL-12 Stimulation of IFN-Gamma Production in NK Cells

Natural Killer (NK) cells secrete interferon gamma (IFN-gamma) in response to IL-12 exposure. Therefore, we measured IFN-gamma production in NK-92 cells (ATCC Accession CRL-2407), a human Natural Killer cell line, in a bioassay to detect the functional activity of scIL-12 designs of the invention.

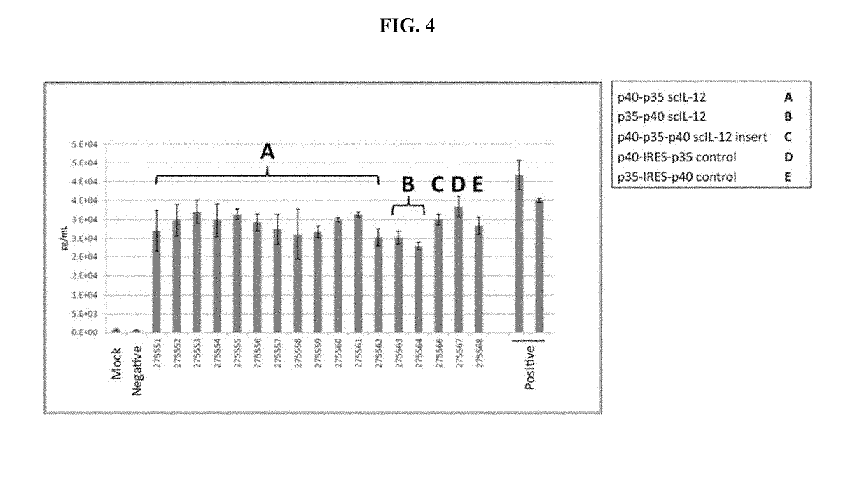

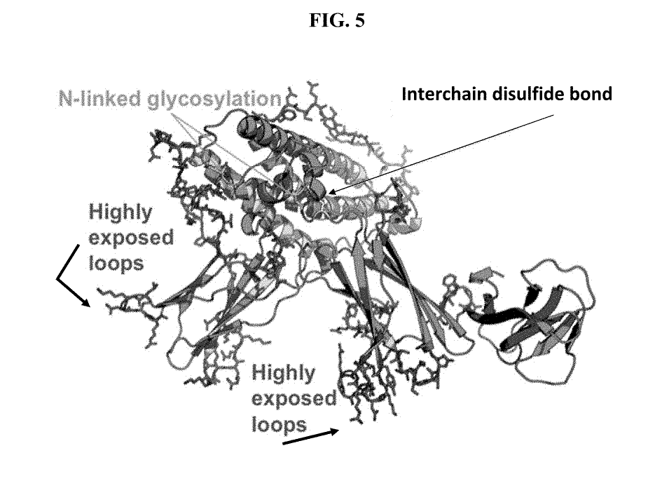

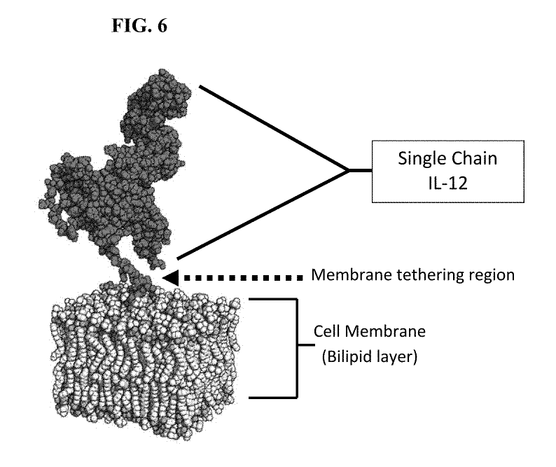

NK-92 cells were cultured according to the manufacturer's instructions using the recommended culture medium (Alpha Minimum Essential medium without ribonucleosides and deoxyribonucleosides, with 2 mM L-glutamine; 1.5 g/L sodium bicarbonate; 0.2 mM inositol; 0.1 mM 2-mercaptoethanol; 0.02 mM folic acid; 100-200 U/ml recombinant IL-2; adjusted to a final concentration of 12.5% horse serum and 12.5% fetal bovine serum). The NK-92 cells were sub-cultured 24-48 hours prior to use in the assay. On the day of the assay, the NK-92 cells were counted by staining with Trypan Blue and seeded into 96-well plates at 5×10⁴ cells per well. CHO-K1/scIL-12 culture supernatants obtained in Example 2 were diluted 1:5 in NK-92 whole growth media and added to the NK-92 cells. Controls included culture supernatants from un-transfected CHO-K1 cells (labeled “Mock” in FIG. 4) and from CHO-K1 cells transfected with plasmid not expressing IL-12 (i.e., CMV-GFP; labeled “Negative” in FIG. 4) as negative controls; and a positive control consisting of commercially available recombinant human IL-12 (R&D Systems), which was tested at 1250 ng/ml or 125 ng/ml (left and right positive controls bars, respectively, in FIG. 4). NK-92 cell culture supernatants were harvested after 48 hours, and diluted 1:10, 1:100, and 1:1000 in R&D Systems Reagent Diluent. The amount of IFN-gamma in the culture medium was determined using the R&D Systems Human IFN-gamma Duoset ELISA kit (Catalog #DY285). Nine samples per vector were analyzed.

Human scIL-12 proteins stimulated human IFN-gamma production in NK-92. Human IFN-gamma expression ranged from 600 pg/mL to 33 ng/mL. See FIG. 4. Similar IFN-gamma levels were observed for the mouse scIL-12 constructs.

Surprisingly, scIL-12 Construct ID 1481273, which exhibited relatively low protein expression levels (see Example 2), demonstrated equivalent activity to recombinant two-chain IL-12 and to p40-p35 single chain constructs in the NK-92 bioassay, suggesting that Construct ID 1481273 may be more active on a per-molecule basis.

### Example 4: Identification of Amino Acid Sequence Modifications for Increasing IL-12 Proteolysis

An analysis of sequences which may be cleaved by a given protease (derived from MEROPS database*) was used to generate a set of starting consensus sequences. These consensus sequences were then cross-compared to general consensus sequences derived from known literature. Potential IL-12 proteolytic sites were subsequently chosen based on accessibility (e.g., hydrophilicity, surface exposure, residue flexibility), the native presence of one or more residues that make up the cleavage site (already present), and a lack of problematic structural or biophysical protein features that might inhibit proteolysis. Not all criteria could be met in every instance; not all sites are amenable to (some or all) mutations matching a consensus sequence, nor, however, are canonical consensus sequences the only sequences applied in a given instance (as it may be desirable to have less than optimal cleavage events/susceptibility to proteolysis). Accordingly, an improved comparative model for human IL-12 was constructed as part of the analysis to effectively place and identify amino acid substitutions to confer proteolytic susceptibility.

Some examples of consensus sequences derived from MEROPS descriptions, which provides a starting range of possibilities from which to guide mutational analysis are:

| | |
|---|---|
| Plasmin: | XXX(RK)\hat{ }XXXX |
| Thrombin: | XX(PAGL)R\hat{ }(SAG)XXX |
| uPA: | XS(GS)(RK)\hat{ }X(RV)XX |
| MMP-2: | XPXX\hat{ }(LI)XXX |

*MEROPS Database: Rawlings ND, Waller M, Barrett AJ, Bateman A., ″MEROPS: the database of proteolytic enzymes, their substrates and inhibitors″. Nucleic Acids Res. 2014 Jan; 42(Database issue): D503-9. doi: 10.1093/nar/gkt953; Epub 2013 Oct 23. PubMed PMID: 24157837; PubMed Central PMCID: PMC3964991.

### Example 5: Characterization of Destabilized IL-12 (DSIL-12) Mutants

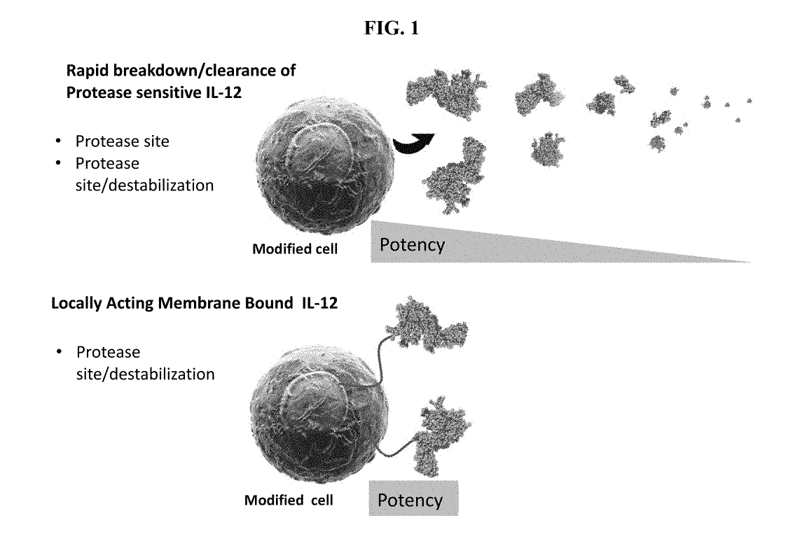

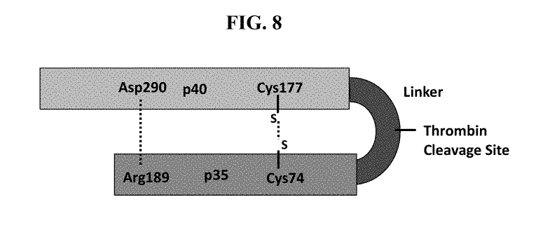

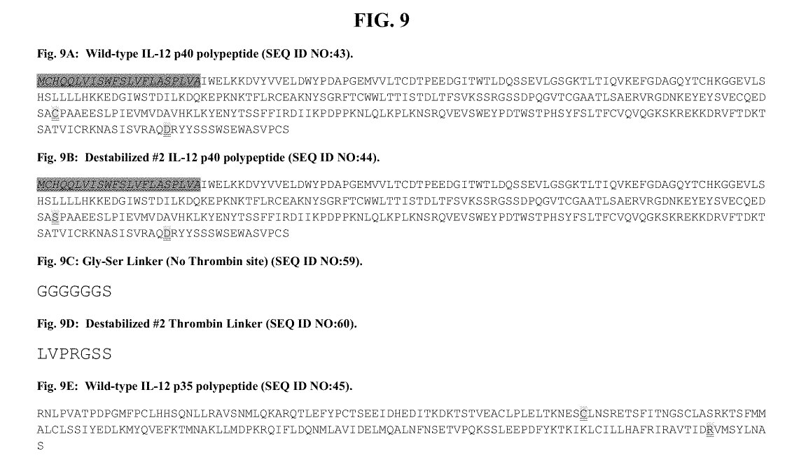

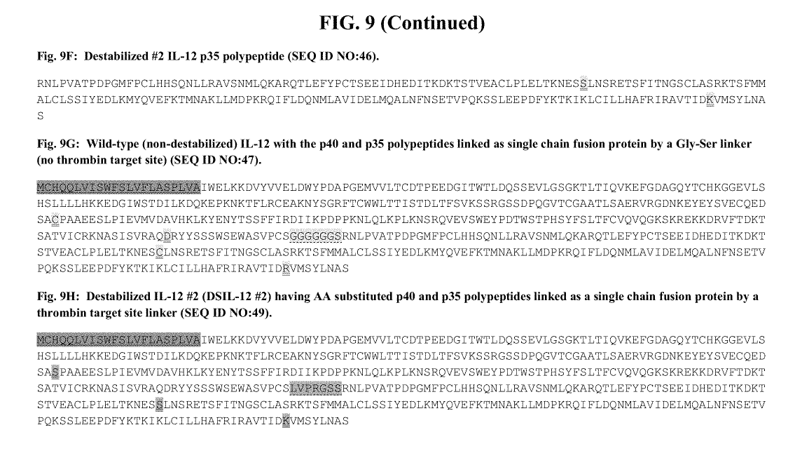

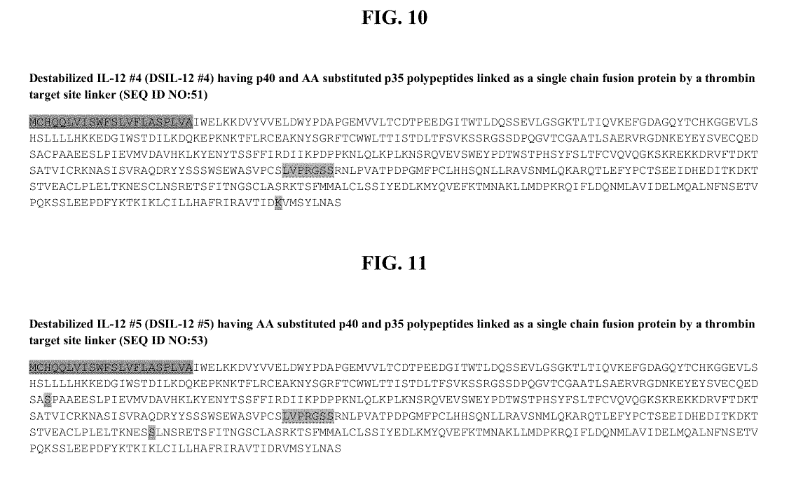

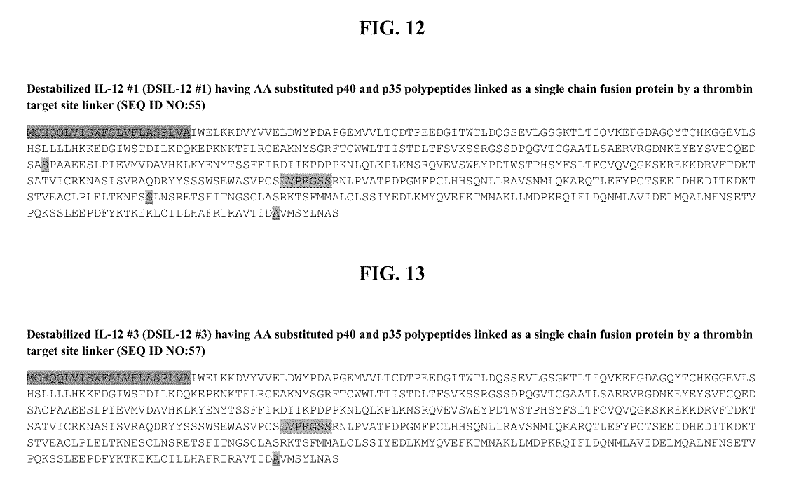

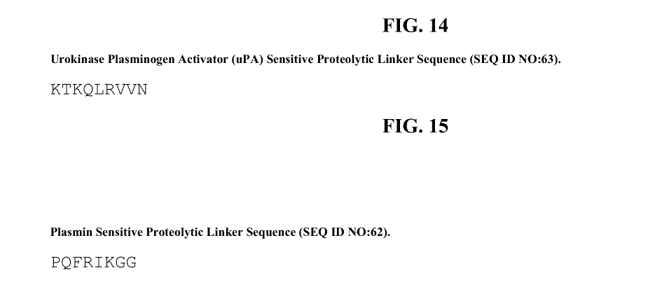

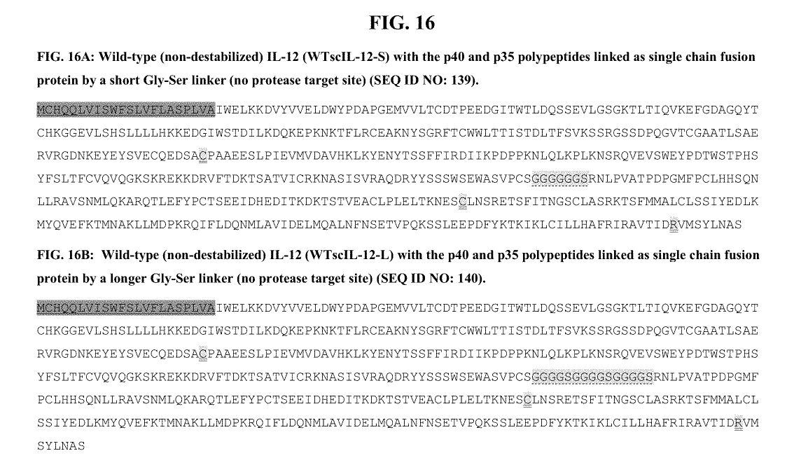

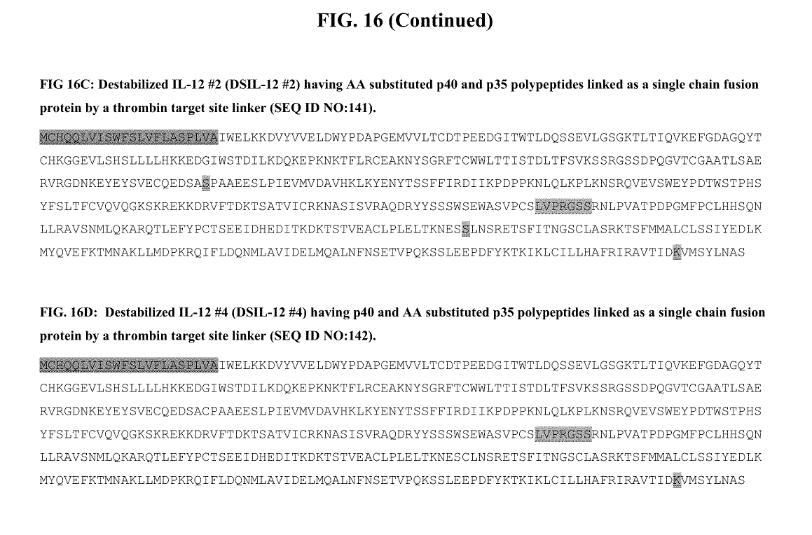

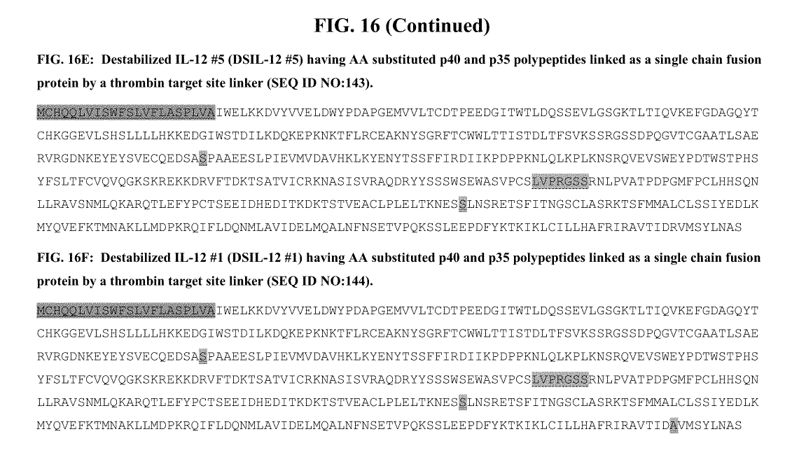

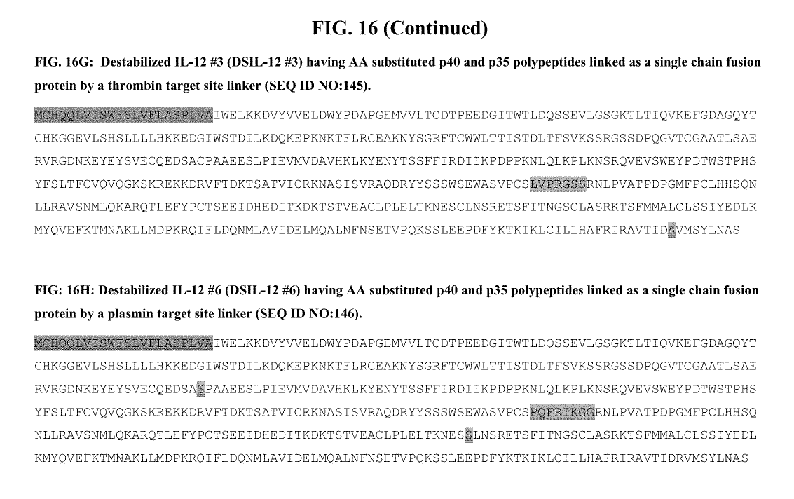

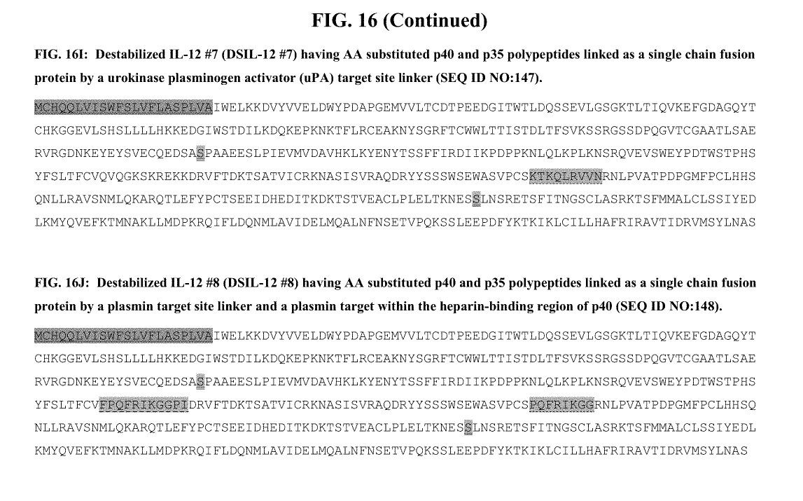

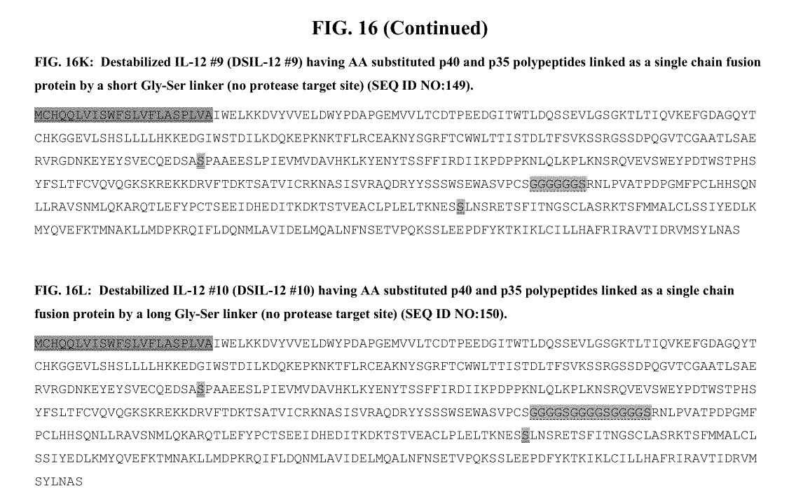

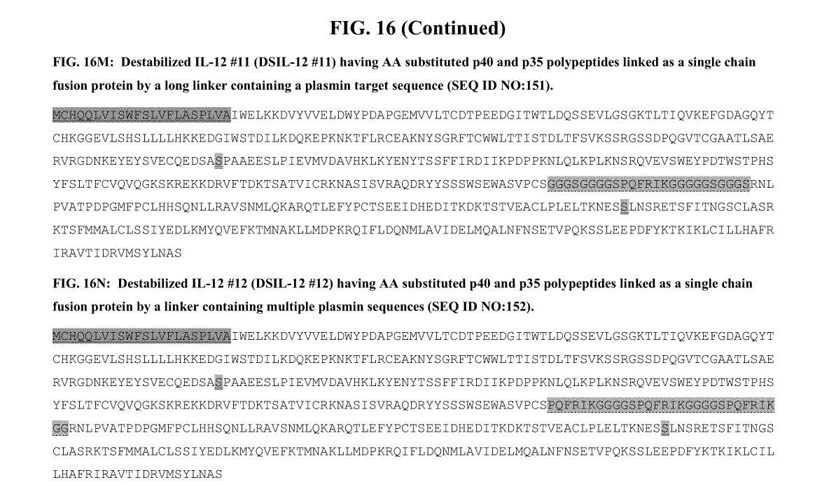

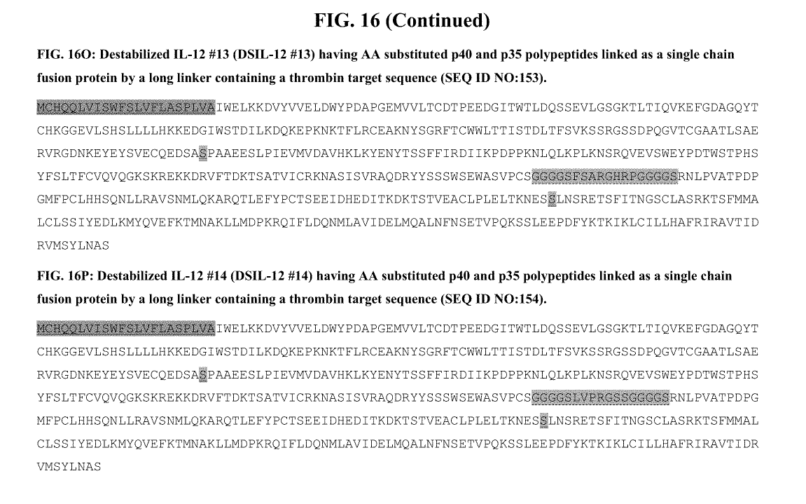

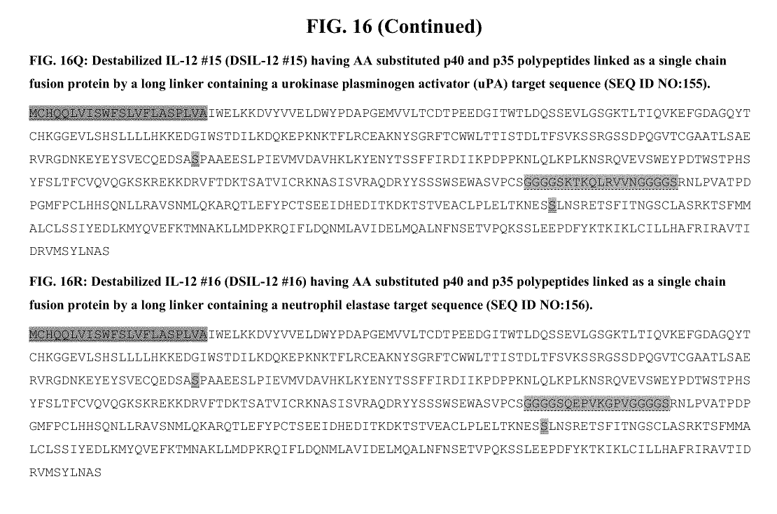

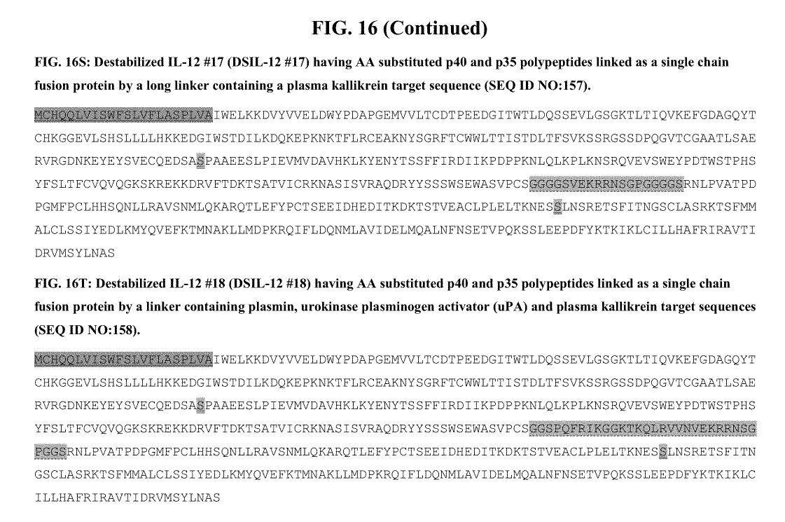

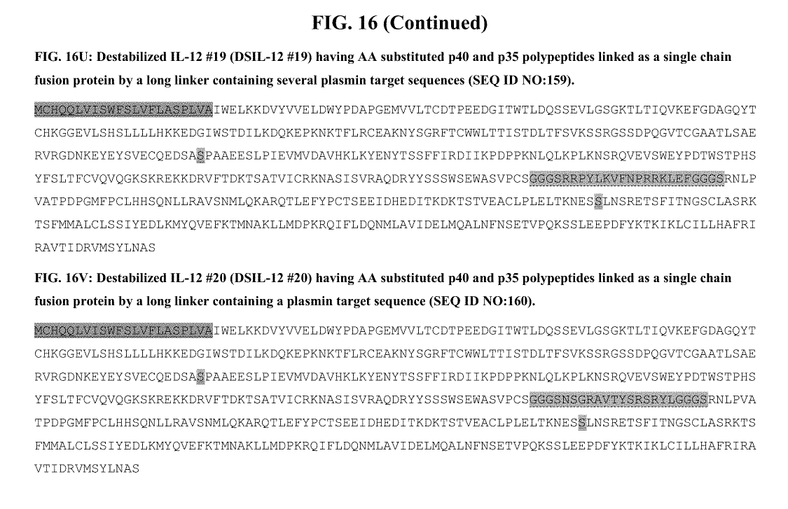

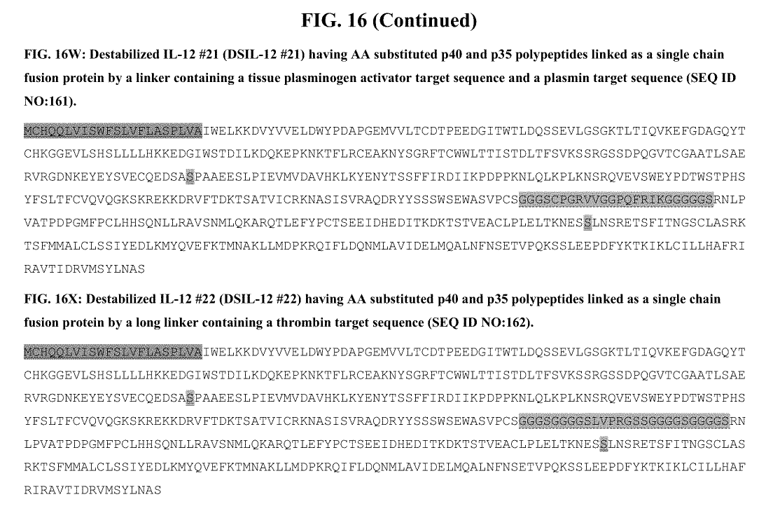

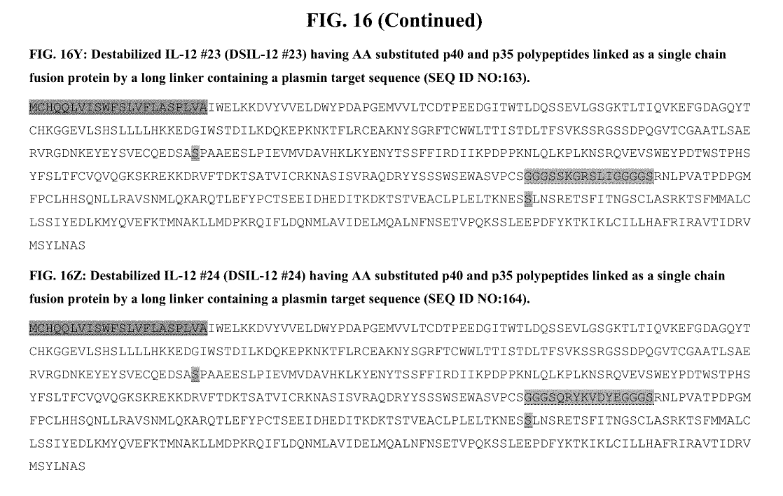

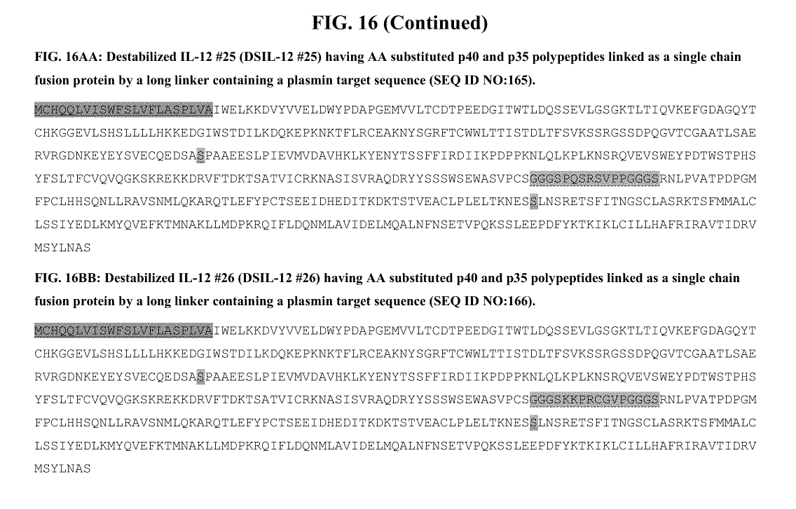

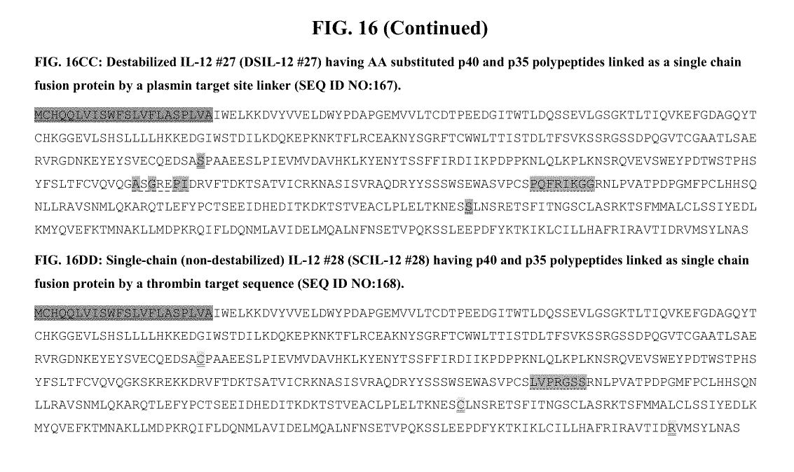

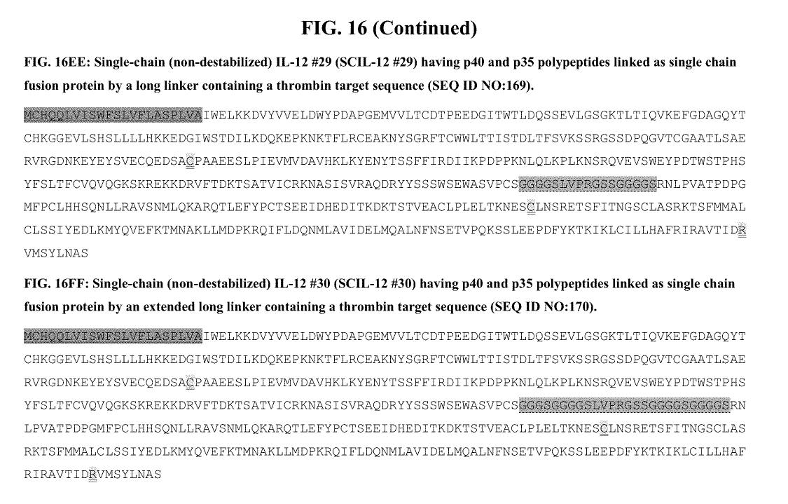

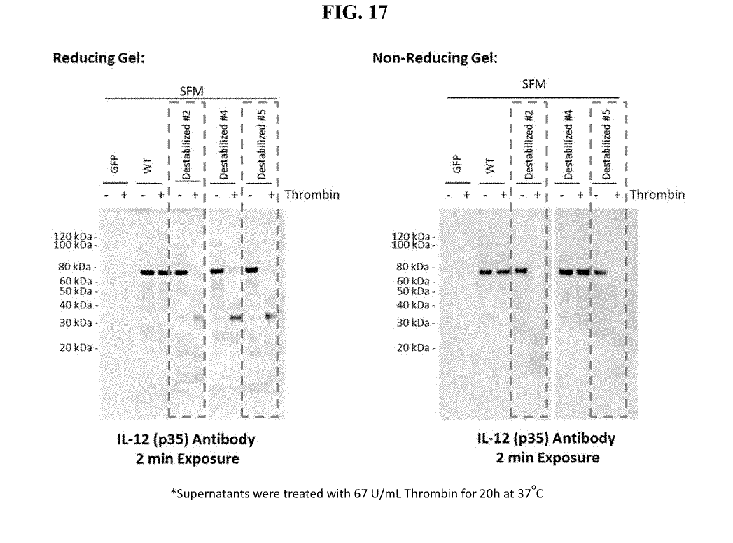

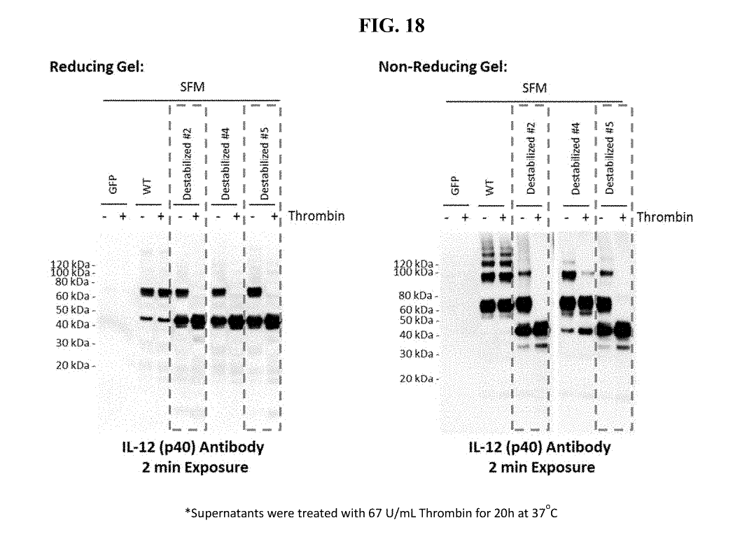

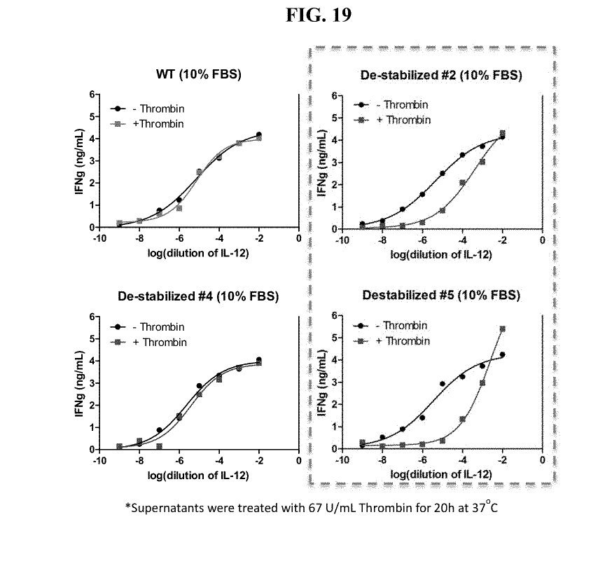

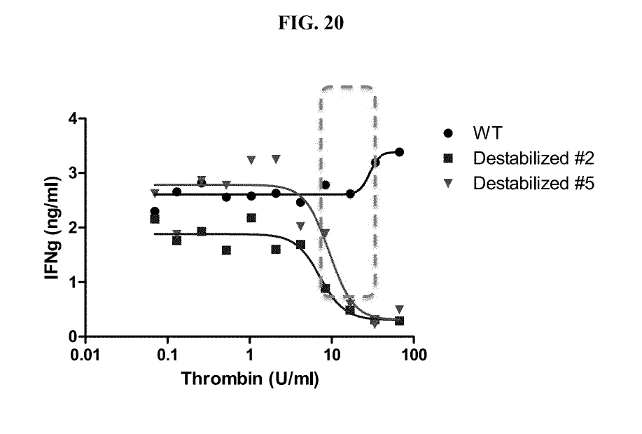

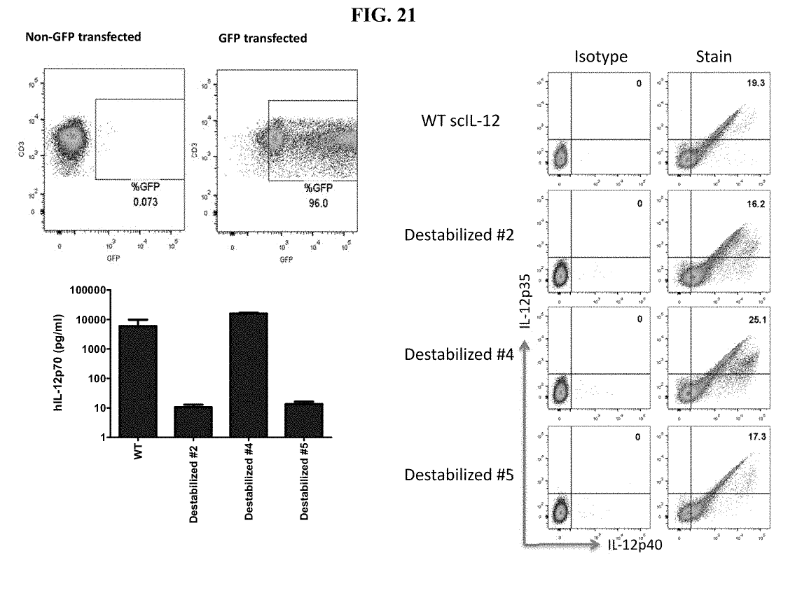

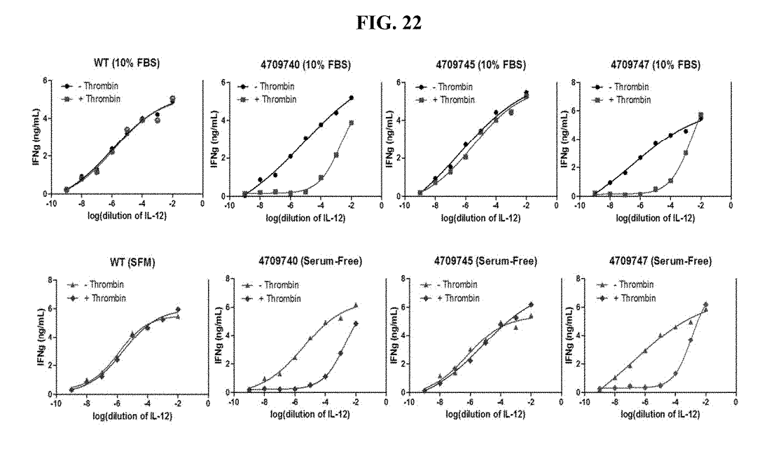

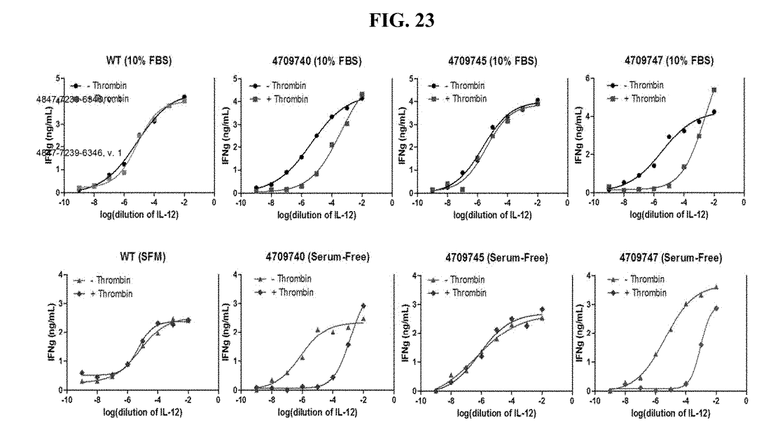

Destabilized IL-12 molecules (DSIL-12) were generated by introducing amino acid changes into naturally-occurring wild-type IL-12 p40 and p35 polypeptide sequences (FIGS. 1 & 2). Multiple destabilized versions of single-chain IL-12 were generated (see e.g., Table 18 and Table 19 and FIGS. 8-13) and tested in in vitro assays. Examples of DSIL-12 mutants are indicated in Table 1.

TABLE 18

| | |
|---|---|
| Destabilized IL-12 | Cys and Arg site mutations in p40 (p40 |
| #1 | C177S) and p35 (p35 C74S/R189A) |
| | subunits combined with a Thrombin site |
| | in the inter-subunit linker. |
| | (SEQ ID NO: 55) |
| Destabilized IL-12 | Cys and Arg site mutations in p40 (p40 |
| #2 | C177S) and p35 (p35 C74S/R189K) |
| | subunits combined with a Thrombin site |
| | in the inter-subunit linker. |
| Destabilized IL-12 | Arg site mutation in p35 subunit (p35 |
| #3 | R189A) combined with a Thrombin site in |
| | the inter-subunit linker. |
| | (SEQ ID NO: 57) |
| Destabilized IL-12 | Arg site mutation in the p35 (p35 R189K) |
| #4 | subunit combined with a Thrombin site in |
| | the inter-subunit linker. |
| Destabilized IL-12 | Cys site mutations in the p40 (p40 C177S) |
| #5 | and p35 (p35 C74S) subunits combined |
| | with a Thrombin site in the inter-subunit |
| | linker. (SEQ ID NO: 53) |

TABLE 19

| | | | | Decreased |
|---|---|---|---|---|
| Destabilized | p40 C177S | p35 C74S | p35 R189X | IL-12 half- |
| Construct | Mutation? | Mutation? | Mutation? | life? |
| #1 | Yes | Yes | Yes | Undetermined |
| | | | (R189A) | |
| #2 | Yes | Yes | Yes | Yes |
| | | | (R189K) | (~11 fold |
| | | | | decrease*) |
| #3 | No | No | Yes | Undetermined |
| | | | (R189A) | |
| #4 | No | No | Yes | Yes |
| | | | (R189K) | (~1.5 fold |
| | | | | decrease*) |
| #5 | Yes | Yes | No | Yes |
| | | | | (~9 fold |
| | | | | decrease*) |

*Compared to wild-type single chain IL-12 (non-destabilized, non-proteolytic linker sequence)

### Example 6: Measuring Half-Life of Modified IL-12 Compositions Via IFN-Gamma Production in NK Cells

Those skilled in the art understand that a number of widely varying methods routinely practiced in the field of the invention could be used to assess (measure, quantify) the reduction in half-life achieved by introducing modifications as described herein into IL-12 polypeptides compared to corresponding non-modified polypeptides. By way of example, one such method is to measure interferon-gamma (IFN-gamma) production by NK cells by comparing samples of modified IL-12 compositions versus non-modified IL-12 compositions which have been exposed to proteases in any number of formats (e.g., contact with recombinant or non-recombinant purified proteinases, contact with animal (e.g., human or non-human) serum samples, contact with plasma (e.g., human or non-human plasma)). The following example illustrates one type of assay which may be used to assess proteolytic susceptibility and half-life of IL-12 polypeptide(s) (compositions) of the invention.

Natural Killer (NK) cells secrete interferon gamma (IFN-gamma) in response to IL-12 exposure. Therefore, IFN-gamma production in NK-92 cells (ATCC Accession CRL-2407), a human Natural Killer cell line, is measured in a bioassay to detect functional activity of IL-12 designs of the invention compared to corresponding non-modified forms of IL-12.

NK-92 cells are cultured according to the manufacturer's instructions using the recommended culture medium (Alpha Minimum Essential medium without ribonucleosides and deoxyribonucleosides, with 2 mM L-glutamine; 1.5 g/L sodium bicarbonate; 0.2 mM inositol; 0.1 mM 2-mercaptoethanol; 0.02 mM folic acid; 100-200 U/ml recombinant IL-2; adjusted to a final concentration of 12.5% horse serum and 12.5% fetal bovine serum). NK-92 cells are sub-cultured 24-48 hours prior to use in the assay. On the day of the assay, the NK-92 cells are counted by staining with Trypan Blue and seeded into 96-well plates at 5×10⁴ cells per well. Modified and non-modified IL-12 compositions are obtained from cell culture supernatants, normalized by dilution as needed to contain the same molar concentrations of modified and non-modified IL-12, exposed to or contacted with a desired test sample comprising one or more proteinases, and subsequently diluted in NK-92 whole growth media which is then added to NK-92 cell cultures. Controls include culture supernatants from cells not producing recombinant IL-12 compositions (e.g., from cells transfected with plasmid not expressing modified or non-modified IL-12 (e.g., CMV-GFP) as negative controls; and positive controls consisting of commercially available recombinant human IL-12 (e.g., from R&D Systems). NK-92 cell culture supernatants are harvested after at various time points, and diluted as needed. The amount of IFN-gamma in the culture medium is determined using, for example, R&D Systems Human IFN-gamma Duoset ELISA kit (Catalog #DY285). Quantities of IFN-gamma production by modified versus non-modified IL-12 compositions exposed to proteases are compared to assess protease susceptibility.

### Example 7: Construction of Additional scIL-12 Fusion Proteins with Variable Linker Length

Additional single chain IL-12 proteins were constructed as outlined in Example 1. The additional single chain IL-12 derivatives are described in Table 15 and Table 16. Each linker sequence contained the thrombin target sequence (Table 15) or the plasmin target sequence (Table 16). The table shows linker sequences of DSIL-12 proteins tested with the thrombin cut site (Table 15) or the plasmin cut site (Table 16) in bold text.

TABLE 15

| SEQ ID | | | |
|---|---|---|---|
| NO: | Name | Linker Sequence | Disulfide Bond |
| 60 | SCIL-12#28 | LVPRGSS | wt |
| 68 | SCIL-12#29 | GGGGSLVPRGSSGGGGS | wt |
| 76 | SCIL-12#30 | GGGSGGGGSLVPRGSSGGGGSGGGGS | wt |
| 67 | DSIL-12#13 | GGGGSFSARGHRPGGGGS | Destabilized |
| 68 | DSIL-12#14 | GGGGSLVPRGSSGGGGS | Destabilized |
| 76 | DSIL-12#22 | GGGSGGGGSLVPRGSSGGGGSGGGGS | Destabilized |

TABLE 16

| SEQ | | |
|---|---|---|
| ID | | |
| NO: | Protein | Linker Sequence |
| 62 | DSIL-12#27 | PQFRIKGG |
| 65 | DSIL-12#11 | GGGSGGGGSPQFRIKGGGGGSGGGS |
| 66 | DSIL-12#12 | PQFRIKGGGGSPQFRIKGGGGSPQFRIKGG |
| 75 | DSIL-12#21 | GGGSCPGRVVGGPQFRIKGGGGGS |
| 72 | DSIL-12#18 | GGSPQFRIKGGKTKQLRVVNVEKRRNSGPGGS |

### Example 8: Linker Length Contributes to Thrombin Sensitivity of Destabilized Single-Chain IL-12 Proteins

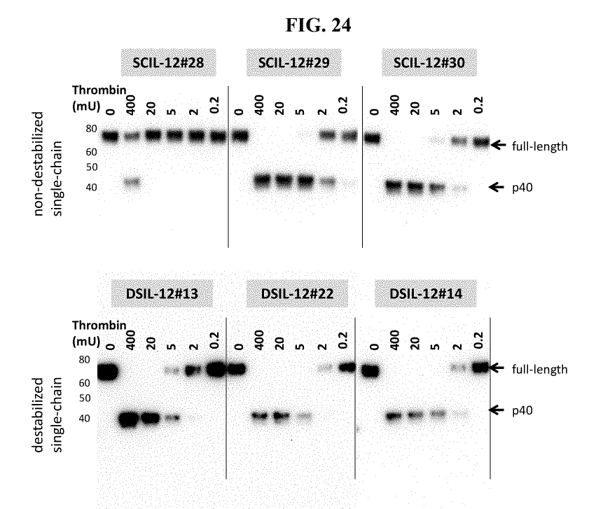

Destabilized single-chain IL-12 proteins containing thrombin target sequences in their linkers were expressed in 293F cells in serum-free medium and supernatants were used for proteolytic digestion assays. Proteins were digested for 1 hour in reactions containing the indicated quantity of human thrombin (489 Units/mg; Millipore). Samples were run on reducing SDS-PAGE followed by Western Blot using mouse anti-human IL-12 p40 antibody (Santa Cruz). See FIG. 24.

The thrombin target sequence LVPRGSS (SEQ ID NO: 60) making up the entire linker in the single chain IL-12 was only partially digested with 400 mU of thrombin (SCIL-12#28) whereas this sequence was completely digested with only 5 mU of thrombin when flanked with spacers in SCIL-12#29, SCIL-12#30, DSIL-12#14 and DSIL-12#22. DSIL-12#13 that contains the thrombin target sequence FSARGHRP (SEQ ID NO: 83) in its linker was less well digested than DSIL-12#14 that contains the LVPRGSS sequence (SEQ ID NO: 60). Therefor the context of the thrombin target sequence in the linker is an important determinant of proteolytic sensitivity. An increased length of linker provides the accessibility required for optimal proteolytic digestion.

### Example 9: Linker Length Contributes to Plasmin Sensitivity of Destabilized Single-Chain IL-12 Proteins

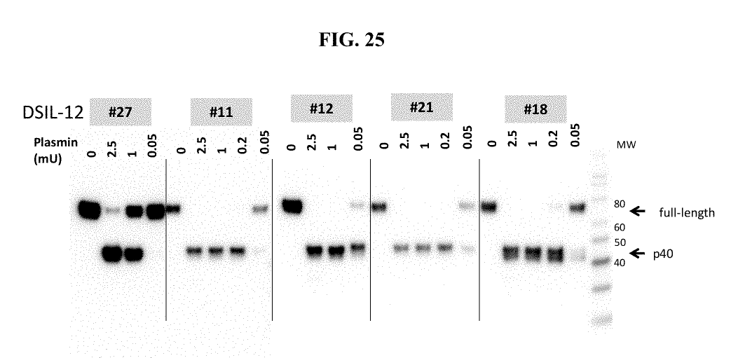

Destabilized single-chain IL-12 proteins containing plasmin target sequences in their linkers were expressed in 293F cells in serum-free medium and supernatants were used for digestion assays. Proteins were digested for 1 hour in reactions containing the indicated quantity of human Plasmin (23.3 Units/mg; Millipore). Samples were run on reducing SDS-PAGE followed by Western Blot using mouse anti-human IL-12 p40 antibody (Santa Cruz). See FIG. 25.

Partial digestion of DSIL-12#11, DSIL-12#12, DSIL-12#21 and DSIL-12#18 was detected with as little as 0.05 mU of plasmin. In contrast, 2.5 mU of plasmin were required for partial digestion of DSIL-12#27. All destabilized single-chain IL-12 proteins tested contain the same plasmin target sequence (PQFRIKGG (SEQ ID NO: 62)) in their linker. Therefor the context of the plasmin target sequence in the linker is an important determinant of proteolytic sensitivity and longer linkers display higher sensitivity to proteolysis.

### Example 10: Digestion of Destabilized Single-Chain IL-12 Proteins in Human Plasma

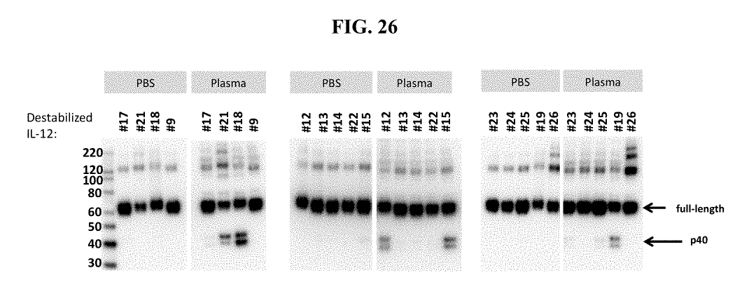

Destabilized single-chain IL-12 proteins were expressed in 293F cells in serum-free medium and supernatants were used for proteolytic digestion assays. Incubations were performed at 37° C. with PBS or 10% heparinized human plasma (Innovative Research) with no additional proteases. At 24 hours, samples were taken, run on non-reducing SDS-PAGE, and analyzed by Western Blot using a mouse anti-human IL-12 p40 antibody (R&D Systems). See FIG. 26.

The data show that certain destabilized single-chain IL-12 proteins are digested in the presence of human plasma. After a 24 hour incubation, bands corresponding to p40 were apparent in certain DSIL-12 samples incubated with plasma, but not those incubated in PBS. Digestion is dependent on the linker as DSIL-12 #9 (containing a linker without protease target sequences) remains full length, while several DSIL-12 configurations with protease target sites in the linker including DSIL-12#15, DSIL-12#18 and DSIL-12#19 display plasma-dependent proteolysis. Therefor plasma proteases are capable of digesting these DS-IL-12 configurations.

### Example 11: Complete Digestion of Destabilized Single-Chain IL-12 Proteins in Human Plasma

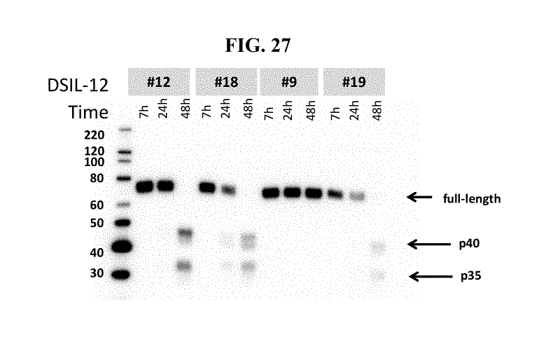

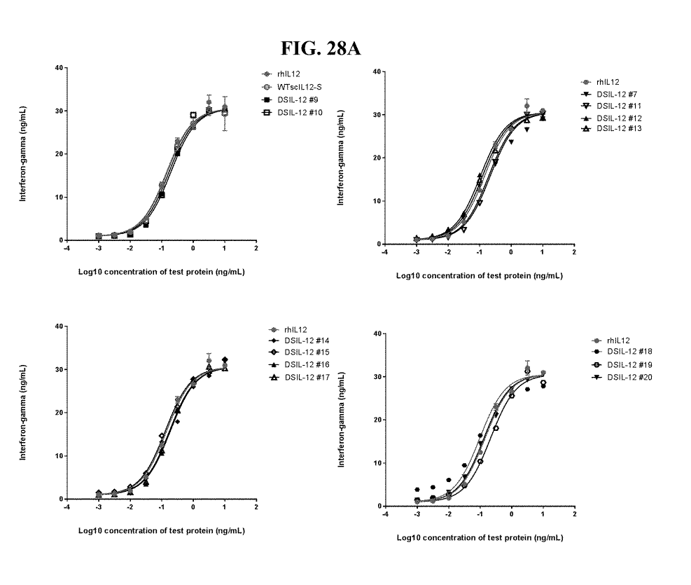

Destabilized single-chain IL-12 proteins were expressed in 293F cells in serum-free medium and supernatants were used for proteolytic digestion assays. Digestions were performed at 37° C. with PBS or 10% heparinized human plasma (Innovative Research) with no additional proteases. At 7, 24 or 48 hours, samples were taken and run on reducing SDS-PAGE. Western blot was performed using polyclonal rabbit anti-IL-12p70 antibody (ABCAM). See FIG. 27.

The data show after 48 hour digestion in 10% human plasma, full-length DSIL-12#12, DSIL-12#18 and DSIL-12#19 proteins are no longer detectable, instead leaving fragments IL-12p35 and IL-12p40. However, DSIL-12 #9 that contains a linker without protease target sequences remains intact. These data demonstrate that plasma protease digestion of DSIL-12 is dependent on the presence of protease-sensitive sequences in the linker.

### Example 12: Bioactivity Assay of Destabilized Single-Chain IL-12 Proteins

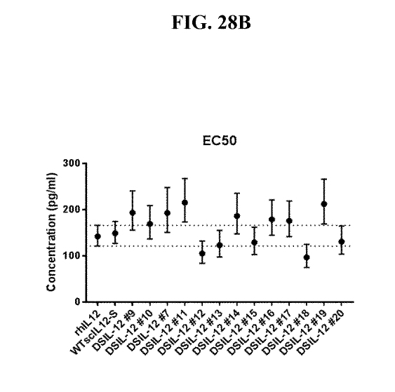

Destabilized single-chain IL-12 proteins and wild-type controls were generated in 293F cells and purified by a combination of ion exchange chromatography, size exclusion chromatography and heparin-column chromatography to approximately 80% purity. Concentrations of proteins were determined by absorption of light at 280 nm. DSIL-12, WTscIL-12 and commercially-obtained recombinant human IL-12p70 (rhIL-12; Peprotech) were serially diluted in tissue culture medium and used to stimulate NK-92 cells in a bioassay. Cells were incubated for 24h and tissue culture supernatants assayed for human Interferon-gamma production. Data were fitted to a four parameter curve. Graphs (A) show dose-response data for DSIL-12 proteins compared with recombinant human IL-12p70 (rhIL-12). Half-maximal effective concentrations (EC50s) were determined for each DSIL-12 along with 95% confidence limits (graph B). See FIG. 28A and FIG. 28B.

The dose-response curves of the wild-type single-chain IL-12 and all destabilized single-chain IL-12 proteins match closely with that generated for the recombinant human IL-12p70 heterodimer indicating that there are no major changes in bioactivity of these DSIL-12 designs. The measurement errors for most DSIL-12 designs overlap with wild-type single chain IL-12 and recombinant human IL-12, suggesting that these are have highly similar if not identical bioactivity. The EC50s of DSIL-12 #11 and DSIL-12 #19 were slightly higher than wild-type and single-chain controls (less than 2-fold) suggesting that a very small loss of bioactivity may occur with these proteins. In conclusion, the modifications of the linkers in the DSIL-12 proteins tested have little influence on their biological activity.

### Example 13: Pharmacokinetic Analysis of Destabilized Single-Chain IL-12 Protein

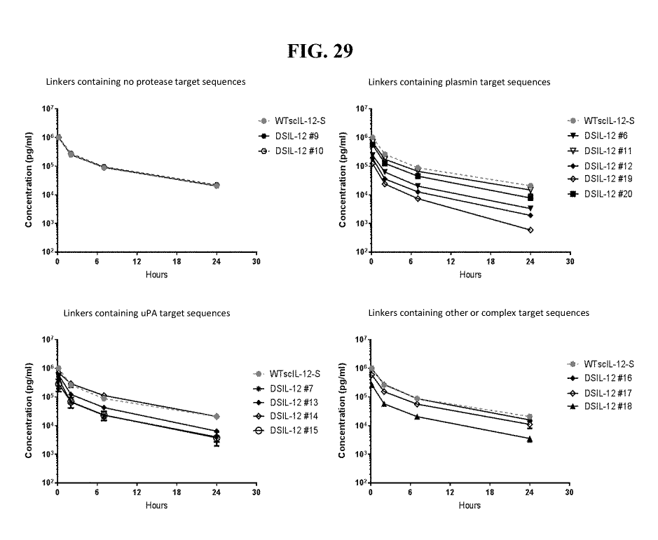

Purified destabilized single-chain IL-12 proteins and wild-type controls were reconstituted in PBS at 50 microgram/mL and 5 micrograms was administered to female C57BL/6 mice via tail vein injection at time 0 with 3 mice per group. At 10 minutes, 2 hours, 7 hours and 24 hours post injection, blood was collected to obtain serum. (A) Serum samples were analyzed for full-length IL-12 using the MSD human IL-12p70 Tissue Culture Kit assay (MesoScale Discovery). Data were normalized based on assay detection efficiency of each purified destabilized single-chain IL-12 or wild-type protein. See FIG. 29A.

Graphs show circulating levels of destabilized single-chain IL-12 (in serum) at each time point tested (n=3+/−S.D.). The wild-type single-chain IL-12 is shown as a comparator (grey circles) on all graphs. Results from each of the destabilized single-chain IL-12 constructs are distributed between the 4 graphs according to protease target sites contained within the linkers. (B) Data from NK92 bioactivity assay on serum from selected groups. Serial dilutions of serum samples or recombinant human IL-12 protein (Peprotech) were used in an NK92 dose-response assay and Interferon-gamma measurements were taken. EC50s derived from this bioassay were used to determine circulating Bioactive Equivalent concentrations of IL-12. See FIG. 29B.

The data demonstrate that pharmacokinetics of destabilized single-chain IL-12 proteins are dependent on the linker sequence. DSIL-12 proteins that contain no protease target sequence in their linkers display identical pharmacokinetics to the wild-type single-chain IL-12. DSIL-12 proteins that contain protease target sequences in their linkers have a variety of pharmacokinetic profiles. DSIL-12 #19 displayed the most altered kinetics, with circulating levels after 10 minutes falling to only 12.2% of the wild-type single-chain IL-12 and circulating levels dropping to 2.9% after 24 hours.

### Example 14: Pharmacokinetic Comparison of Protease Target DSIL-12 #19 with Destabilized Single-Chain IL-12 Containing a Gly-Ser Linker

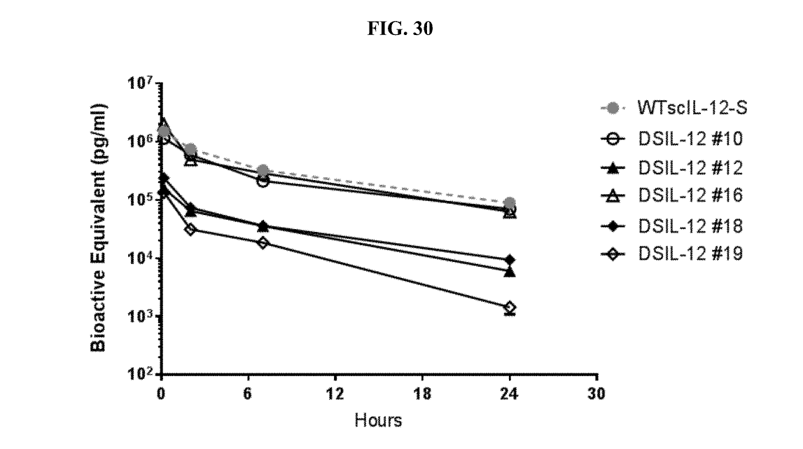

Purified destabilized single-chain IL-12 proteins were reconstituted in PBS and 5 or 20 micrograms was administered to female C57BL/6 mice via tail vein injection at time 0. At 10 minutes, 2 hours, 7 hours and 24 hours post injection, blood was collected to obtain serum or plasma. Serum samples were analyzed for full-length IL-12 using the MSD human IL-12p70 Tissue Culture Kit assay (MesoScale Discovery). Data were normalized based on assay detection efficiency of each purified destabilized single-chain IL-12. The circulating levels of DSIL-12 #10 and DSIL-12 #19 in serum and plasma are shown at each at each time point tested (n=2+/−S.D.). The percentages of DSIL-12 #19 recovered from serum compared with control DSIL-12 #10 were tabulated for each time point. See FIG. 30.

Circulating levels of DSIL-12 #19 (containing proteolytic target sequences in its linker) were drastically reduced compared with circulating levels of control DSIL-12 #10 (that has no protease target sequence in its linker). Even if 4-fold higher doses of DSIL-12 #19 are delivered, detectable levels are below the control after 10 minutes and down to 15% of control after 24 hours. No difference was observed between measurements in plasma and serum samples, confirming that the samples are representative of circulating blood at the given time. In conclusion, the data demonstrate that pharmacokinetics of destabilized single-chain IL-12 proteins depend on the linker sequence and proteolytic sensitivity of this linker sequence may be modified to decrease the protein's half-life.

### Example 15: Development and Manufacturing of Cancer Immunotherapies and Controlled Gene Programs

It is contemplated that embodiments of the invention include the following.

Development of Peripheral Blood Autologous T Cell Therapies with Endogenous Anti-Tumor Activity Genetically Modified with Controlled IL-12 for Use in the Immunotherapy of Patients with Metastatic Cancer

Interleukin 12 (IL-12) was the first recognized member of a family of heterodimeric cytokines that includes IL-12, IL-23, IL-27, and IL-35. IL-12 and IL-23 are pro-inflammatory cytokines important for development of T helper 1 (Th-1) and T helper 17 (Th-17) T cell subsets, while IL-27 and IL-35 are potent inhibitory cytokines. IL-12 can directly enhance the activity of effector CD4 and CD8 T cells as well as natural killer (NK) and NK T cells. Preclinical studies in murine tumor treatment models demonstrate powerful antitumor effects following the systemic administration of IL-12. In humans, however, attempts to systemically administer recombinant IL-12 resulted in significant toxicities including patient deaths and limited efficacy.

The treatment of patients with cell populations expanded ex vivo is called adoptive cell transfer (ACT). Cells that are infused back into a patient after ex vivo expansion traffic to the tumor and mediate its destruction. ‘Preparative lymphodepletion’—the temporary ablation of the immune system in a patient with cancer—can be accomplished using chemotherapy alone or in combination with total-body irradiation, and the addition of this step is associated with enhanced persistence of the transferred T cells. Moreover, the combination of a lymphodepleting preparative regimen with ACT and administration of T cell growth factor IL-2 can lead to prolonged tumor eradication in patients with metastatic melanoma or other tumor histologies who have exhausted other treatment options.

Recent studies involving exomic sequencing of human melanomas have indicated the presence of a large number of mutational events, enabling the targeting of non-synonymous mutations that result in the creation of new epitopes. The inherent genetic instability of tumors generates many potential tumor-associated antigens, which may result from somatic single-base mutations within gene-coding regions, from mutations in stop codons that extend open reading frames, from frameshift mutations, or from gene rearrangements that lead to the production of fusion proteins, among other mechanisms.

It is hypothesized that the clinical responses following adoptive transfer of ex vivo expanded tumor-specific T cells is the result of bypassing local suppression of the tumor microenvironment. The TILs are dissociated from immunosuppressive cell populations, such as myeloid-derived suppressor cells (MDSCs) and possibly exposed to lower levels of immunosuppressive cytokines during this early period in culture. Expansion of such T cell populations ex vivo are challenged by high patient cellular loading requirements and adjunctive use of cytokines to enable anti-tumor activity. IL-12 is a potent cytokine, which, when genetically engineered into tumor-specific T cells, can facilitate significant clinical response.

It has previously been observed (in patients with metastatic melanoma treated in a cell-dose escalation trial of autologous TILs transduced with a gene encoding a single chain IL-12 driven by a nuclear factor of activated T cells promoter (NFAT.IL-12)) that administration of 0.001-0.1×10⁹ NFAT.IL-12 transduced TILs resulted in a single objective response (5.9%). However, at doses between 0.3-3×10⁹ cells, 63% of patients exhibited objective clinical responses. However, these responses tended to be short and the administered IL-12 producing cells rarely persisted after one month. Moreover, increasing cell doses were associated with high serum levels of IL-12 and gamma-interferon (IFN-γ) as well as clinical toxicities including liver dysfunction, high fevers and sporadic life threatening hemodynamic instability.

Using a ligand (veledimex) controlled RTS promoter-driven IL-12 gene program, preliminary data suggest dose proportional expression of IL-12, and cessation of ligand administration is associated with reversal of moderate to severe adverse events.

Native human IL-12 p70 has a reported terminal half-life in the range of 13 to 19 hours. Reducing plasma accumulation of IL-12 may improve systemic tolerability while maintaining local potency. Protease-sensitive IL-12 variants and membrane tethered IL-12 variants are screened for biofunction and protease cleavage in vitro. Evaluation of variants under RTS controlled expression in anti-tumor lymphocytes in preclinical models is used to determine clinical efficacy.

Ad-RTS-hIL-12 with veledimex activator ligand has been the subject of clinical investigation in patients with solid tumor malignancies. Preclinical studies have demonstrated ligand dose-dependent expression of mouse and human IL-12 with this gene construct. Ongoing clinical trials in patients with advanced melanoma and breast cancer employ an adenovirally-delivered IL-12 (Ad-RTS-hIL-12), under RTS control, by injection into the tumors followed by oral administration of the ligand.

In a Phase-1, 3+3 dose escalation study, 14 patients with unresectable stage III/IV melanoma received 10¹² adenovirus particles (Ad-RTS-hIL-12) intratumorally. Ad-RTS-hIL-12 was administered on the first day of up to six 21-day cycles and escalating doses of veledimex (activator ligand/INXN-1001) were administered orally on days 1 to 7 of each cycle. Dose escalation studies were completed spanning all 14 patients. One death unrelated to study drug was secondary to septicemia. One patient at the 160 mg dose had stable disease for 20 weeks. Dose cohorts ≥100 mg coincided with a 4-fold median increase from baseline in peak serum levels of IL-12 and IFN-γ compared with lower dose cohorts. Flow cytometric analyses of PBMCs revealed 7-fold (≥100 mg dose cohorts) median increases from baseline in peak levels of absolute numbers of CD3+ and CD8+ T-cells.

Design of short-acting IL-12 expands upon and improves biofunctional control relative to other human and murine single chain IL-12 designs. Single-chain candidates demonstrating a potency profile similar to the wild type (wt) are engineered with a series of mutations to add proteolytic cleavage sites to the molecule. Several proteases containing overlapping and promiscuous cleavage sites are considered in order to maximize potential for rapid degradation. Energy analysis using protein structure analytical software is performed to review and triage designs. In addition to the protease sensitive sites, alternative approaches to reduce scIL-12 systemic diffusion through various membrane-anchoring strategies are also assessed.

In sum, T cells with endogenous anti-tumor activity can recognize tumor-specific neo-epitopes derived from the products of the mutated cancer genome. It is hypothesized that clinical response following adoptive transfer of ex-vivo expanded tumor-specific T cells is the result of bypassing local suppression of the tumor microenvironment. Expansion of such T cell populations ex vivo are challenged by high patient cellular loading requirements and adjunctive use of cytokines to enable anti-tumor activity. Interleukin-12 is a potent cytokine, that when genetically engineered into tumor-specific T cells, can facilitate impressive clinical response with significantly reduced cell loading. However, this efficacy is accompanied by unacceptable systemic toxicities. This example describes application of molecular engineering tools for integration of spatial and temporal control of interleukin-12 in tumor specific T cells for use in patients with solid tumor malignancies characterized by high mutation frequency.

Embodiments of the invention include spatial and temporal control of Interleukin-12 (IL-12) in T cell therapies for the treatment of patients with solid tumor malignancies. Viral compositions may be used to deliver spatially and temporally controlled IL-12; also including regulated expression of IL-12 via oral activator ligands such as, but not limited to, veledimex. Endogenous T cells are transduced using viral compositions to evaluate safety and effectiveness in relevant animal models. Tumor infiltrating lymphocytes (TILs) are adapted according to clinically-acceptable manufacturing protocols to enable peripheral blood-derived lymphocyte expansion directed against tumor-specific antigens, followed by viral transduction with viral compositions for investigation of therapeutic effects.

### IL-12 Viral Compositions for Spatial and Temporal Control in Peripheral Blood Lymphocytes are Generated

A ligand controlled RHEOSWITCH THERAPEUTIC SYSTEM® (RTS®) inducible gene switch platform is inserted into a lentiviral backbone to express single chain IL-12 (sclL-12) variant(s). Basal expression and dynamic range of the RTS® system in human lymphocytes is optimized with established internal analytical methods to maximize temporal control in comparison to constitutive vector systems and NFAT-scIL-12 constructs. In parallel, variants of scIL-12 are screened for plasma proteinase sensitivity in vitro and transmembrane versions are screened for protein shedding from the surface. Potency of scIL-12 variants are confirmed using the natural killer cell (such as NK-92 cells) IFN-γ bioassays in co-cultures. Murine versions of sufficiently bioactive scIL-12 constructs are subsequently tested in syngeneic tumor models. Viral preparations of lead candidates are used for dose selection pharmacology and preclinical safety assessment in relevant animal models and T cell populations are compared with NFAT IL-12 viral constructs.

Current TIL Protocols are Adapted to Peripheral Blood Lymphocyte Expansion Against Tumor Mutation Specific Antigens with Viral Transduction

A mutation-exome sequencing minigene presentation process is adapted to peripheral blood mononuclear cell expansion and viral transduction in vitro. One objective includes ensuring product sterility, removal of process-related impurities, establishment of tandem minigenes and HLA expression in supportive cell substrates (or autologous APC/syn-mRNA), cell expansion, T cell phenotypic analysis, specification setting, and future technology transfer.

Expression Controlled scIL-12 Candidates are Compared with Native IL-12 for Comparative Safety Assessment in Representative Models for Lead Candidate Selection

Lead viral stocks are used in testing cellular products for pre-clinical safety assessment in comparison with the existing NFAT-driven scIL-12.

Maximum Tolerated Dose of Cell Product and Veledimex Activator Ligand is Established in Patients with Suitable Tumor-Specific Mutations

Peripheral blood lymphocytes are harvested from patients with solid tumor malignancies and tumors are biopsied for comparative exome sequencing and HLA-based peptide presentation analysis. Constructs are assembled (from patients exhibiting suitable mutation profiles for presence of tandem minigenes) for cell product manufacturing and subsequent systemic viral transduction. Systemic administration follows a lymphodepleting chemotherapy regimen. The MTD (maximum tolerated dose) may be determined through a matrix of limited cell dose escalations followed by oral activator ligand (e.g., veledimex) dose escalation.

### Generation of IL-12 Viral Stocks for Spatial and Temporal Control of Expression in Lymphocytes and Development of Spatially Controlled IL-12 to Compliment Veledimex Activator Ligand Temporal Control in Tumor Specific Lymphocytes.

Candidate screening is performed by evaluating experimental scIL-12 expression in transiently transfected CHO-DG44 or HEK293F cells. Multiple molecular designs are screened for potency and decreased half-life. Expression and quantification of various scIL-12 designs is followed by NK-92 IFN-γ potency assays to provide a baseline of activity. Designs not retaining at least about 50%, 60%, 70%, 80% or 90% or more wild-type activity or that demonstrate clear expression problems are excluded from further testing. Molecules having activity are subjected to in vitro assessment of proteolytic sensitivity. Designs are added to plasma spiked with proteases and subjected to both detection of protease cleavage by western blot and biofunctional analysis by NK-92 IFN-γ assay. Candidates demonstrating desirable levels of protease sensitivity are assessed in secondary screens as inducible vector constructs.

An alternative approach to a short-lived (protease sensitive) IL-12 is proposed an IL-12 molecule (scIL-12 or heterodimeric IL-12) anchored to a T-cell surface (e.g., TM-scIL-12; Pan 2012, Bozeman 2013). Construction of a limited number of variants as lentiviral constructs under control of the RTS® inducible gene expression system followed by cell-based assay where TM-scIL-12 expressing T-cells are co-cultured with NK-92 cells to quantify IFN-γ production as a functional readout on the local effects of TM-scIL-12. Shedding of bioactive IL-12 from the surface is used as a secondary screen to monitor and assess protein release from lymphocytes. Desirable candidates and their murine counterparts are incorporated in lentiviral stocks under RTS® expression platform control for pharmacology and for safety assessment.

### REFERENCES

auf dem Keller U, Prudova A, Gioia M, Butler G S, Overall C M. A statistics-based platform for quantitative N-terminome analysis and identification of protease cleavage products. Mol Cell Proteomics. 2010 May; 9(5):912-27. PMID: 20305283.
Bozeman E N, Cimino-Mathews A, Machiah D K, Patel J M, Krishnamoorthy A, Tien L, Shashidharamurthy R, Selvaraj P. Expression of membrane anchored cytokines and B7-1 alters tumor microenvironment and induces protective antitumor immunity in a murine breast cancer model. Vaccine. 2013 May 7; 31(20):2449-56. doi:10.1016/j.vaccine.2013.03.028. Epub 2013 Mar. 28. PubMed PMID: 23541884.
Carra G, Gerosa F, Trinchieri G. Biosynthesis and posttranslational regulation of human IL-12. J Immunol. 2000 May 1; 164(9):4752-61. PubMed PMID: 10779781.
Castellino F J, Powell J R. Human plasminogen. Methods Enzymol. 1981; 80 PtC:365-78. Review. PMID: 6210827.
Chang J Y. Thrombin specificity. Requirement for apolar amino acids adjacent to the thrombin cleavage site of polypeptide substrate. Eur J Biochem. 1985 Sep. 2; 151(2):217-24. PubMed PMID: 2863141.
Ellis V. u-Plasminogen Activator. Handbook of Proteolytic Enzymes, 2 ed. (Barrett, A. J., Rawlings, N. D. & Woessner, J. F.), p. 16′7′7-1683, Elsevier, London (2004).
Heeb M J, Griffin J H. Activated protein C-dependent and independent anticoagulant activities of protein S have different structural requirements. Blood cell Mol Dis. 2002 September-October; 29(2):190-9. PMID 12490286.
Kushlinskii N E, Timofeev Y S, Solov'ev Y N, Gerstein E S, Lyubimova N V, Bulycheva I V. Components of the RANK/RANKL/OPG System, IL-6, IL-8, IL-16, MMP-2, and Calcitonin in the Sera of Patients with Bone Tumors. Bull Exp Biol Med. 2014 August; 157(4):520-3. PubMed PMID: 25110097.
Nagarajan S, Reddy B S, Tsibouklis J. In vitro effect on cancer cells:synthesis and preparation of polyurethane membranes for controlled delivery of curcumin. J Biomed Mater Res A. 2011 Dec. 1; 99(3):410-7. doi: 10.1002/jbm.a.33203. Epub 2011 Aug. 23. PubMed PMID: 22021188.
Pan W Y, Lo C H, Chen C C, Wu P Y, Roffler S R, Shyue S K, Tao M H. Cancer immunotherapy using a membrane-bound interleukin-12 with B7-1 transmembrane and cytoplasmic domains. Mol Ther. 2012 May; 20(5):927-37. doi: 10.1038/mt.2012.10. Epub 2012 Feb. 14. PubMed PMID: 22334018.
Rossano R, Larocca M, Riviello L, Coniglio M G, Vandooren J, Liuzzi G M, Opdenakker G, Riccio P. Heterogeneity of serum gelatinases MMP-2 and MMP-9 isoforms and charge variants. J Cell Mol Med. 2014 February; 18(2):242-52. PubMed PMID: 24616914.
Schilling O, Overall C M. Proteome-derived, database-searchable peptide libraries for identifying protease cleavage sites. Nat Biotechnol. 2008 June; 26(6):685-94. PubMed PMID: 18500335.
Shohrati M, Haji Hosseini R, Esfandiari M A, Najafian N, Najafian B, Golbedagh A. Serum matrix metalloproteinase levels in patients exposed to sulfur mustard. Iran Red Crescent Med J. 2014 March; 16(3):e15129. PubMed PMID: 24829780; PubMed Central PMCID: PMC4005442.
Spassov V Z, Yan L. pH-selective mutagenesis of protein-protein interfaces: in silco design of therapeutic antibodies with prolonged half-life. Proteins 2013 April; 81(4):704-14. PMID: 23239118.
Vucemilo T, Skoko M, Sarcević B, Puljiz M, Alvir I, Turudie T P, Mihaljević I. The level of serum pro-matrix metalloproteinase-2 as a prognostic factor in patients with invasive ductal breast cancer. Coll Antropol. 2014 March; 38(1):135-40. PubMed PMID: 24851607.
Yoon C, Johnston S C, Tang J, Stahl M, Tobin J F, Somers W S. Charged residues dominate a unique interlocking topography in the heterodimeric cytokine interleukin-12. EMBO J. 2000 Jul. 17; 19(14):3530-41. PMID: 10899108.

The invention claimed is:

1. A composition comprising an IL-12 p40 polypeptide and an IL-12 p35 polypeptide wherein at least one of said p40 or p35 polypeptides comprise at least one non-naturally occurring substitution mutation and at least one non-naturally occurring proteolytic target sequence.

2. The composition of claim 1 wherein the half-life or biological activity of said composition is decreased compared to a corresponding wild-type IL-12 composition.

3. The composition of claim 1 or 2, wherein p40 and p35 are covalently linked as a single chain fusion protein.

4. The composition of claim 3, wherein a linker sequence used to covalently link p40 and p35 polypeptides comprises a proteolytic target sequence.

5. The composition of any one of claims 1 to 4, wherein said composition comprises one or more amino acid substitutions which increase the rate of proteolysis of said composition compared to the rate of proteolysis of a corresponding IL-12 composition not having said one or more amino acid substitutions.

6. The composition of any one of claims 1 to 5, wherein said composition is a heterodimer of p40 and p35 polypeptides.

7. The composition of claim 5, wherein the corresponding non-modified IL-12 composition is a heterodimer of human IL-12 p40 and human IL-12 p35 polypeptides.

8. The composition of any one of claims 1 to 7, wherein said composition is a topologically manipulated single chain IL-12 polypeptide.

9. The compositions of any one of claims 1 to 8, wherein said composition comprises a p40 polypeptide which comprises any one or more amino acid substitutions selected from the group consisting of:
K126L
K124G/K126L
K124A/K126L
K124S/K126L
K124G/N125G/K126L
K124A/N125A/K126L
M45L
N248L
K247A/N248L
L246A/K247A/N248L
L246S/K247A/N248L
A172P
A172P/T174A
D40A/P42L
G161P/D164L
K126L
K124G/K126L
K124A/K126L
K124S/K126L
K124G/N125G/K126L
K124A/N125A/K126L
M45L
D287S
K302S/N303S
V180S
K280L/S281V/K282P/E284G/K285S
S176L/A177V/E178P/V180T/R181S
K280L/S281V/K282P/E284G/K285V
S176L/A177V/E178P/V180S/R181S
N248S/S249G
K282G/K285V
S249G
K282G/K307V
wherein these substitution positions correspond to amino acid positions as shown in SEQ ID NO: 2.

10. The composition of any one of claims 1 to 9, wherein said composition comprises a p35 polypeptide which comprises any one or more amino acid substitutions selected from the group consisting of:
Q186L
S215L
Y223L
K214P
K214P/S216A
C144P/S147L
C144P/L145S/S147L
G142R/R148G
K149S
K149A
E135S
Q186S
S216R
D111A/K112R
Q213R/K214L/S215R/S216A
A146V/S147P/K149G/T150S/S151K
N132V/S133P/E135G/T136S/S137K
S147P/K149I/T150I/S151K
N132F/S133P/E135G/S137K
N77I/L78P/S83R
T210L/Q213R/K214G
R148G/K149R
N207S/S208G/E209R
E209G/T210R
wherein these substitution positions correspond to amino acid positions as shown in SEQ ID NO: 4.

11. The composition of any one of claims 1 to 8, wherein said IL-12 composition comprises a topologically manipulated single chain IL-12 polypeptide which comprises any one or more amino acid substitutions selected from the group consisting of:
K126L
K124G/K126L
K124A/K126L
K124S/K126L
K124G/N125G/K126L
K124A/N125A/K126L
M45L
N248L
K247A/N248L
L246A/K247A/N248L
L246S/K247A/N248L
Q426L
S455L
Y463L
A172P
A172P/T174A
K454P
K454P/S456A
C384P/S387L
C384P/L385S/S387L
D40A/P42L
G161P/D164L
D287S
K302S/N303S
V180S
G382R/R388G
K389S
K389A
E375S
Q426S
S456R
D351A/K352R
Q453R/K454L/S455R/S456A
K280L/S281V/K282P/E284G/K285S
S176L/A177V/E178P/V180T/R181S
A386V/S387P/K389G/T390S/S391K
N372V/S373P/E375G/T377S/S378K
K280L/S281V/K282P/E284G/K285V
S176L/A177V/E178P/V180S/R181S
S365P/K367I/T368I/S369K
N372F/S373P/E375G/S377K
N317I/L319P/S323R
T450L/Q453R/K454G
K280L/S281V/K282P/E284G/K285S
N248S/S249G
K282G/K285V
S249G
K282G/K285V
R388G/K389R
N447S/S448G/E449R
E449G/T450R
wherein these substitution positions correspond to amino acid positions as shown in SEQ ID NO:10.

12. An interleukin-12 (IL-12) composition wherein said composition has been modified to comprise a membrane linking (tethering/anchoring/binding) moiety and, wherein said IL-12 composition comprises one or more amino acid substitutions which increase the rate of proteolysis of said composition compared to the rate of proteolysis of a corresponding IL-12 composition not having said one or more amino acid substitutions.

13. The composition of claim 12, wherein said IL-12 composition comprises a heterodimer of p40 and p35 polypeptides.

14. The composition of any one of claim 12 or 13, wherein the corresponding non-modified IL-12 composition is a heterodimer of human IL-12 p40 and human IL-12 p35 polypeptides.

15. The composition of any one of claims 12 to 14, wherein said IL-12 composition comprises a single chain IL-12 polypeptide.

16. The composition of any one of claims 12 to 15, wherein said membrane anchoring, linking, or tethering) moiety is selected from the group consisting of: a covalent membrane surface linking moiety, a hydrophobic membrane surface linking moiety, a hydrophillic membrane surface linking moiety, an ionic membrane surface linking moiety, an integral membrane polypeptide, and a transmembrane polypeptide.

17. The composition in any one of claims 12 to 16, wherein IL-12 expression is inducibly regulated by a gene switch.

18. The composition of any one of claims 12 to 19, wherein IL-12 expression is inducibly regulated by a gene switch.

19. The composition of claim 20, wherein said gene switch is an ecdysone receptor-based (EcR-based) switch.

20. The composition of any one of claims 12 to 21, wherein said IL-12 is expressed by a modified T cell.

21. The composition of any one of claims 12 to 22, wherein said IL-12 is expressed by a modified T cell.

22. A method of treating a cancer or an immune system disorder comprising administering a therapeutically useful amount of the composition of any one of claims 12 to 23.

23. A method of treating a cancer or immune system disorder comprising administering a therapeutically useful amount of the composition of any one of claims 12 to 23.

24. The composition of claim 4, wherein the linker sequence covalently links p40 on the N-terminus of the linker, and p35 on the C-terminus of the linker.

25. The composition of claim 4, wherein the linker sequence covalently links p35 on the N-terminus of the linker, and p40 on the C-terminus of the linker.

26. The composition of claim any one of claims 1 to 27, wherein the single chain fusion protein comprises a signal peptide.

27. The composition of any one of claims 1 to 10 or 12 to 28, wherein the composition comprises a membrane-anchoring sequence.

28. The composition of any one of claim 1 to 11 or 26-29, wherein the at least one non-naturally occurring substitution mutation disrupts a disulfide bond.

29. The composition of any one of claims 1 to 11 or 26 to 30, wherein the non-naturally occurring proteolytic target sequence comprises SEQ ID NO: 60, 63, or 62.

30. The composition of any one of claims 1 to 9, wherein the composition comprises a polypeptide of SEQ ID NO: 40, 46, 49, 51, or 53.

31. The composition of any one of claims 1 to 11 or 24 to 30, wherein the proteolytic target sequence comprises a thrombin target sequence or a plasmin target sequence.

32. The composition of claim 31, wherein the proteolytic target sequence comprises a thrombin target sequence or a plasmin target sequence, and at least four amino acids on N-terminal side of the thrombin target sequence or a plasmin target sequence.

33. The composition of claim 31, wherein the proteolytic target sequence comprises a thrombin target sequence or a plasmin target sequence, and at least four amino acids on C-terminal side of the thrombin target sequence or a plasmin target sequence.

34. The composition of claim 31, wherein the proteolytic target sequence comprises a thrombin target sequence or a plasmin target sequence, at least four amino acids on N-terminal side of the thrombin target sequence or a plasmin target sequence, and at least four amino acids on C-terminal side of the thrombin target sequence or a plasmin target sequence.

35. The composition of claim 31, wherein the proteolytic target sequence comprises a thrombin target sequence or a plasmin target sequence, and four to twenty amino acids on N-terminal side of the thrombin target sequence or a plasmin target sequence.

36. The composition of claim 31, wherein the proteolytic target sequence comprises a thrombin target sequence or a plasmin target sequence, and four to twenty amino acids on C-terminal side of the thrombin target sequence or a plasmin target sequence.

37. The composition of claim 31, wherein the proteolytic target sequence comprises a thrombin target sequence or a plasmin target sequence, four to twenty amino acids on N-terminal side of the thrombin target sequence or a plasmin target sequence, and four to twenty amino acids on C-terminal side of the thrombin target sequence or a plasmin target sequence.

38. The composition of any one of claims 1 to 37, wherein the composition comprises a linker sequence selected from the group consisting of SEQ ID NOS 60, 68, 76, 67, 62, 65, 66, 75 and 72.

39. A polynucleotide or polynucleotides encoding the composition of any one of claims 1 to 38.

40. A method of treating a cancer or an immune pathology or disorder comprising administering a therapeutically effective amount of the composition of any one of claims 1 to 39.
